SEQUENCE LISTING (46 sequences)

SEQ ID NO: 1 - DNA, 2659 bp, Homo sapiens
  misc_feature 35: n = a, t, g, or c
gccccatcgctgcctcgcaggaggcgaacgccgcngaccccagcggagccgcggcccagccttgatcccccaaccccgggggctggcatgagcggcccctcggcggcaccgtggggcggtggagtcgcctgtcgcctccgcctgatccccggcctgtcggccgaccccacctcgccaaccgaggcggaccgcggagtgtgcgaacgaccccaccgctgctttctcctcccccagatcacgcaccccagctccggaagatggggaactgcctcaaatcccccacctcggatgacatctccctgcttcacgagtctcagtccgaccgggctagctttggcgaggggacggagccggatcaggagccgccgccgccatatcaggaacaagttccagttccagtctaccacccaacacctagccagactcggctagcaactcagctgactgaagaggaacaaattaggatagctcaaagaataggtcttatacaacatctgcctaaaggagtttatgaccctggaagagatggatcagaaaaaaagatccgggagtgtgtgatctgtatgatggactttgtttatggggacccaattcgatttctgccgtgcatgcacatctatcacctggactgtatagatgactggttgatgagatccttcacgtgcccctcctgcatggagccagttgatgcagcactgctttcatcctatgagactaattgagccagggtctcttatctgacttcaagtgaaccaccattttggtggttttgatcttttgtcactgagcccaaagagccagggattaggaattaagatcgtgcacaaaagtttccttaaaattcctggatggctgcagatgttgggggaaaaagtacgtgatattttagaaacttagtgggaaaagtaggatggtatttttatgtaaagccttgacccaatgtttaaaaatataattgtatttagatcttgttattgctccagtacataggaattgtgtaaagtgttaacagcagctgtatttgtttaaattgtgtgtattgaagattaggaaaaagatagtagttatttttcctaaatgaaataactttcttctcttccccttccccacccgaattcttttctgaagttgctggcatttgggtcaaggttttattaaaagctacattttataacactggcacacacaaaaaagtagttttaagcttgtttgcacagttctttttttccattggaaatggaattcattgccttaggtctttttaaatagtgtattattatcgttggggctggctctatgcttgaaaaccagtttatttataacctgttataagtgctatattctgtttgcagttaggaaatgcagaattcaaagtgatctcctagcttgtaagcaaactgagatgcactatcccttttctataaaaaataagttaatgtgtcaagaaaccaactctattaaggtggggtttaatattaccctttcctatgtgttttatctaattattttggttgttaatatggtgataatggaaagtcaagttaaattttaaatattaagaattctgatttattgagattgaattatgccaccacgtttatgtaaaaatgaaggtggcaccgtggtgagacctaatgagaaatagttactcagttgtaaaaattttgatttattctctttcttctgacctccttgcctcttgtcttgaaccatagcaaaaggatactgcatctctcattactgtagtgctgaggttattgaagttatacaaaacacatctcagtctctgtttcttggaaaggtatctattacatcctgctagctgactgacaaaactaagcagggagaataaagataattgtattttatgttttgcacacaaacgcagaatttgtataaccatatgacttcatagttgtgatctcaaaaaaagaaggaatttctcctttgtttcttgcagttaatgtaagaatactttaaatctctaagcttctgaagtgttagaggtagagatggtctagtaaagatgtagtagtaatgttttatccatttagcatgtgtttattttttcatatgtactcaaaggtgacttattggttcacctcagtgatattacagctaaaaaaatcattcattagcaaaaggaaaagtggtctcaacctaacatcagaagtgtttcttattattattttatattgagttgaatattgaactctaacagttttctacatacaaaacacagtgtcatgaaggttattcataattgcattatagaggaatgtagtatgtcataagtactttgtaaagatttgacattcaactgtagtatccatatgttgcttaaatttccttatgagccccatgatggaaagacttaaagatgaatttgagaaaaattgaaagaaattagattatcaggttctgttaaattgttacatgtatcttgcttaaatttctgtttattaatttatatccacccaagtacataaagcaaatttggaggaaacaactgaagttgtgcaatattttctgataattgctttttttattcttgtgttttctacttaaacataatgtctgtgtcatcaagtattatagtcagacttttctttttttctagattgttaaaattggcaaatgaacttttttaaaaatcaaa

SEQ ID NO: 2 - protein, 154 aa, Homo sapiens
MGNCLKSPTSDDISLLHESQSDRASFGEGTEPDQEPPPPYQEQVPVPVYHPTPSQTRLATQLTEEEQIRIAQRIGLIQHLPKGVYDPGRDGSEKKIRECVICMMDFVYGDPIRFLPCMHIYHLDCIDDWLMRSFTCPSCMEPVDAALLSSYETN

SEQ ID NO: 3 - DNA, 2177 bp, Homo sapiens
ttgtggagccggcgggcgggccgtttgtctctccttcctgcgcctctttgcgtgctgggaggcgggccgcagggtagctgttggcgccgccgcgtttctgggcctggccaactcacgtgaccgacgcgggctctgaagcggtgggccgcaccgaggcggcggcggcggcgacgactgcagctcgggaggtagaggcctggcgagggacgggccggctgccctctcggacggccgcggcggagggcaaaaatggcggaggcttcggcggccggggcggactcgggcgccgctgtagccgcccaccggtttttctgccacttttgcaagggcgaggtcagccccaaactaccggaatatatatgtcccagatgtgaatcaggctttattgaagaagtgacagatgattccagttttttaggtggtggcggcagtcggatagacaataccacaacaacacattttgcagagctttggggccatttggatcacacgatgttttttcaagattttagaccctttctaagtagcagtccactggaccaagataatagagccaatgaaaggggtcaccagactcacactgacttctggggagcaagacctccacggttgccattgggtcggagatacagatctcgaggaagttctcgtcctgacagatctccagctattgaaggaatactacaacacatctttgcaggattctttgcaaattctgccattcctggatctccacaccctttttcctggagcgggatgctgcactccaaccctggggactatgcctggggtcagacagggcttgatgccattgtaacccagcttttaggacaactggaaaacacaggccctcccccagctgacaaggaaaagatcacatctcttccaacagtgacagtaactcaggaacaagttgatatgggtttagagtgtccagtatgcaaagaagattacacagttgaagaggaagtccggcagttaccttgcaatcacttctttcacagcagttgtattgtgccgtggctagaactgcatgacacatgtcctgtatgtaggaagagcttaaatggtgaggactctactcggcaaagccagagcactgaggcctctgcaagcaacagatttagcaatgacagtcagctacatgaccgatggactttctgaagctaaagaccacacctgaatcagggctgtggtaatcatcttaccatagctggaaattgtatcaaaacaaaaaattagtagatggatttaggaatatgtaagaaactcaacacataatataaatgcaatgaatgtttttcttctttaaatttaaagttagtatctacagatggaattgtatctacaaccaaatgcctcttatccctgaattcagagtgataattttataagtgtgaaacttaattatgtagggctccccccgtctgaatagaattaattccttaaagtctagttagggtcctgctgtctgtcatgttgccttgtaacggatgtttccacctccttctccaacctctaccccaccattagtgtattttactataaaaacagtggaaccacagccctaaagtcctgctgatataaagtccttttgtcttaattgtatttaaaaaaaaaaaaaaaactactcttgatcacattagctatgaggcgaggtcaaattcaggtatctaagactaatgattttgttttgtcttgatccccagagagcaaatcaaagaaaaattacagcaagaagagaaaagtggtttatcatgtgtgctggtttcttatttaattttttgtttttggaaaggagtctcggtctgtcgctaggctggagtgcacggccgatcttggtcatggaaactctgcctctgagtcaagcgatctctactcagctcggtggcttgggccccgcctttgtgaccatgcccataatttctggtacccgcaaaaggggttccccggtgtggccagaggtcgaatctctgactgtgtccggcgtccaccccaaaagggggggaaggggacacagccgaacgcatataaaaaagaaaaaagagaagcgcacaacaaaacagagagagacagaaaaagcgacacagagaagagagaacagacaacaacaagaaaaaaaagaaccataagagagaacaagaccaatacagaccaagcttggactagaaagcacc

SEQ ID NO: 4 - protein, 304 aa, Homo sapiens
MAEASAAGADSGAAVAAHRFFCHFCKGEVSPKLPEYICPRCESGFIEEVTDDSSFLGGGGSRIDNTTTTHFAELWGHLDHTMFFQDFRPFLSSSPLDQDNRANERGHQTHTDFWGARPPRLPLGRRYRSRGSSRPDRSPAIEGILQHIFAGFFANSAIPGSPHPFSWSGMLHSNPGDYAWGQTGLDAIVTQLLGQLENTGPPPADKEKITSLPTVTVTQEQVDMGLECPVCKEDYTVEEEVRQLPCNHFFHSSCIVPWLELHDTCPVCRKSLNGEDSTRQSQSTEASASNRFSNDSQLNDRWTF

SEQ ID NO: 5 - DNA, 1319 bp, Homo sapiens
ctgcgccggcgccgccgggaagcgctaggcctggtccctcttcctaggatagcgttgcgcgcatgcgcctttgacgagtgagccggggagccatggacaactgtttggcggccgcagcgctgaatggggtggaccgacgttccctgcagcgttcagcaaagctggctctagaagtgctggagagggccaagaggagggcggtggactggcatgccctggagcgtcccaaaggctgcatgggggtccttgcccgggaggcgccccacctagagaaacagccggcagccggcccgcagcgcgttctcccgggagagagagaagagagacccccaacccttagtgcttccttcagaacaatggctgaattcatggactatacttcaagtcagtgtgggaaatattattcatctgtgccagaggaaggaggggcaacccatgtctatcgttatcacagaggcgagtcgaagctgcacatgtgcttggacatagggaatggtcagagaaaagacagaaaaaagacatcccttggtcctggaggcagctatcaaatatcagagcatgctccagaggcatcccagcctgctgagaacatctctaaggacctctacatagaagtatatccagggacctattctgtcactgtgggctcaaatgacttaaccaagaagactcatgtggtagcagttgattctggacaaagcgtggacctggtcttccctgtgtgatgttgaccatcactgccatcacatcacctttttttaagtagtaagaataaagccactgtatgattctcttaatagctatacattaatcctgtttttagtgctgactgggtcagccttccgggaactggagtctgtctctttcagtgcttttttgtttgtttggttggttgttttttgagacagtctcactctgttgcccaggctggagtgcagtggcgtgatctcggctcactgcaagttccgcctcccgggttcacaccattctcctgcctcagcctcccgagtagctggcactacaggcacccgccaccatgcccggctattttttttgtatttttagtagagacggggtttcaccatgttggccaggatggtctcgatctcttgacctcgtgatccacccaccttggcctcccaaagtgttgggattacaggcgtgagccaccgcgcccggcctcagtgccttttttaacttgagggtgtagaggtcctccacgcttgtttgcctgaaagtaatataatgatgctgtctgaacaggttttactgcttgctttccaagtaaaggttaattatgataataaagagatttggaa

SEQ ID NO: 6 - protein, 210 aa, Homo sapiens
MDNCLAAAALNGVDRRSLQRSAKLALEVLERAKRRAVDWHALERPKGCMGVLAREAPHLEKQPAAGPQRVLPGEREERPPTLSASFRTMAEFMDYTSSQCGKYYSSVPEEGGATHVYRYHRGESKLHMCLDIGNGQRKDRKKTSLGPGGSYQISEHAPEASQPAENISKDLYIEVYPGTYSVTVGSNDLTKKTHVVAVDSGQSVDLVFPV

SEQ ID NO: 7 - DNA, 251 bp, Homo sapiens
ttgatcaaaaacctttattctggtcctcatcattcaggaagccacttatgcagaacactttcttcatctctgctctaagcagcagcagctagtccaggcttgatatggaagacagacagaaatagtggaagaccttgcagagaagagctaattgatgagactctgcttatcccggcataggtgaaggcataacaggaagagtgtgtgtaagtgcaaagactccgttcttcaaagctgaggcctgtgcagag

SEQ ID NO: 8 - protein, 84 aa, Homo sapiens
  misc_feature 26, 60, 64, 70, 84: Xaa = STOP
LIKNLYSGPHHSGSHLCRTLSSSLLXAAAASPGLIWKTDRNSGRPCREELIDETLLIPAXVKAXQEECVXVQRLRSSKLRPVQX

SEQ ID NO: 9 - DNA, 154 bp, Homo sapiens
ttgttggtggctgaatacattttggaagagagtttttcatcttagagattggtgaacaagtgtgagggtgtgagaaactcacagaatacaaatttgcctgtatgttttgtgggttttttttttcctttcaagatgttttctatttctaaattaa

SEQ ID NO: 10 - protein, 52 aa, Homo sapiens
  misc_feature 15, 52: Xaa = STOP
LLVAEYILEESFSSXRLVNKCEGVRNSQNTNLPVCFVGFFFPFKMFSISKLX

SEQ ID NO: 11 - DNA, 600 bp, Homo sapiens
gcggccgggcttttcttgaactgttagtttttttgaagtagttttttcatgtttaatttgtatttgtaaaaaaacaaaaagcaaaaaaattcccaaaacccagataacaaccagagcaaaactgttgtgccttctatttatctttgatttcagtcttggcaattgtttaaaaaaaaaatctagatttgttttattaggttcagagtatgtggggaattatagaatccctctttcatcactttgtgtatgtcttttgttaacatatttgttatgccttattctaaaattgagtctcaaactggaatgcctttgaagacagatgcttctatagaggttctttgacctaaatagttcagcatttgtatttttattctggtatctaatcagattcctaatcatagcccgtaagaaggaatgttactttaatattggactttgctcatgtgctcgtgtccgcatttttttttttcttaaaatcatagccatatggtaaattttctattttgttatggttctcttttattgatgggcatgcagtgggtgttacttggaaatggccaatttttattaaaatatttctggaagaaaaaaaaaaaaaaa

SEQ ID NO: 12 - protein, 200 aa, Homo sapiens
  misc_feature 9, 12, 23, 49, 61, 74, 114, 117, 136, 142, 158: Xaa = STOP
AAGLFLNCXFFXSSFFMFNLYLXKNKKQKNSQNPDNNQSKTVVPSIYLXFQSWQLFKKKIXICFIRFRVCGELXNPSFITLCMSFVNIFVMPYSKIESQTGMPLKTDASIEVLXPKXFSICIFILVSNQIPNHSPXEGMLLXYWTLLMCSCPHFFFSXNHSHMVNFLFCYGSLLLMGMQWVLLGNGQFLLKYFWKKKKKK

SEQ ID NO: 13 - DNA, 351 bp, Homo sapiens
tttggtctttatgtgcttaaataacgctgaattataattagccacacaaataatgagagttttattttttttttttggctcactccaaatcagcctgttaaggtatatttccttctacagcctttcctgattttgcatgttctcattcccaaagtagtctaccttagtttacactcaaaggtagcacttgttgaaactacatgacagaaacaggctgcaaaggtggacaaggggaagcataatgtccctcttgtcttgcataactcagtgccacacacagaacccacattttctgagacattatcttcattatagagccgtttgattccatcatagaagtcatgcggccgc

SEQ ID NO: 14 - protein, 117 aa, Homo sapiens
  misc_feature 8, 10, 68, 105, 112: Xaa = STOP
FGLYVLKXRXIIISHTNNESFIFFFWLTPNQPVKVYFLLQPFLILHVLIPKVVYLSLHSKVALVETTXQKQAAKVDKGKHNVPLVLHNSVPHTEPTFSETLSSLXSRLIPSXKSCGR

SEQ ID NO: 15 - protein, 41 aa, Homo sapiens
CVICMMDFVYGDPIRFLPCMHIYHLDCIDDWLMRSFTCPSC

SEQ ID NO: 16 - protein, 5 aa, Artificial
  Description of Artificial Sequence domain
PPPPY

SEQ ID NO: 17 - skipped

SEQ ID NO: 18 - DNA, 1238 bp, Homo sapiens
ctgcgccggcgccgccgggaagcgctaggcctggtccctcttcctaggatagcgttgcgcgcatgcgcctttgacgagtgagccggggagccatggacaactgtttggcggccgcagcgctgaatggggtggaccgacgttccctgcagcgttcagcaaagctggctctagaagtgctggagagggccaagaggagggcggtggactggcatgccctggagcgtcccaaaggctgcatgggggtccttgcccgggaggcgccccacctagagaaacagccggcagccggcccgcagcgcgttctcccgggagagaaatattattcatctgtgccagaggaaggaggggcaacccatgtctatcgttatcacagaggcgagtcgaagctgcacatgtgcttggacatagggaatggtcagagaaaagacagaaaaaagacatcccttggtcctggaggcagctatcaaatatcagagcatgctccagaggcatcccagcctgctgagaacatctctaaggacctctacatagaagtatatccagggacctattctgtcactgtgggctcaaatgacttaaccaagaagactcatgtggtagcagttgattctggacaaagcgtggacctggtcttccctgtgtgatgttgaccatcactgccatcacatcacctttttttaagtagtaagaataaagccactgtatgattctcttaatagctatacattaatcctgtttttagtgctgactgggtcagccttccgggaactggagtctgtctctttcagtgcttttttgtttgtttggttggttgttttttgagacagtctcactctgttgcccaggctggagtgcagtggcgtgatctcggctcactgcaagttccgcctcccgggttcacaccattctcctgcctcagcctcccgagtagctggcactacaggcacccgccaccatgcccggctattttttttgtatttttagtagagacggggtttcaccatgttggccaggatggtctcgatctcttgacctcgtgatccacccaccttggcctcccaaagtgttgggattacaggcgtgagccaccgcgcccggcctcagtgccttttttaacttgagggtgtagaggtcctccacgcttgtttgcctgaaagtaatataatgatgctgtctgaacaggttttactgcttgctttccaagtaaaggttaattatgataataaagagatttggaa

SEQ ID NO: 19 - protein, 183 aa, Homo sapeins [sequence omitted]

SEQ ID NO: 20 - DNA, 2314 bp, Homo sapeins
ctgcgccggcgccgccgggaagcgctaggcctggtccctcttcctaggatagcgttgcgcgcatgcgcctttgacgagtgagccggggagccatggacaactgtttggcggccgcagcgctgaatggggtggaccgacgttccctgcagcgttcagcaaagctggctctagaagtgctggagagggccaagaggagggcggtggactggcatgccctggagcgtcccaaaggctgcatgggggtccttgcccgggaggcgccccacctagagaaacagccggcagccggcccgcagcgcgttctcccgggagagaaatattattcatctgtgccagaggaaggaggggcaacccatgtctatcgttatcacagaggcgagtcgaagctgcacatgtgcttggacatagggaatggtcaggctgagaacatctctaaggacctctacatagaagtatatccagggacctattctgtcactgtgggctcaaatgacttaaccaagaagactcatgtggtagcagttgattctggacaaagcgtggacctggtcttccctgtgtgatgttgaccatcactgccatcacatcacctttttttaagtagtaagaataaagccactgtatgattctcttaatagctatacattaatcctgtttttagtgctgactgggtcagccttccgggaactggagtctgtctctttcagtgcttttttgtttgtttggttggttgttttttgagacagtctcactctgttgcccaggctggagtgcagtggcgtgatctcggctcactgcaagttccgcctcccgggttcacaccattctcctgcctcagcctcccgagtagctggcactacaggcacccgccaccatgcccggctattttttttgtatttttagtagagacggggtttcaccatgttggccaggatggtctcgatctcttgacctcgtgatccacccaccttggcctcccaaagtgttgggattacaggcgtgagccaccgcgcccggcctcagtgccttttttaacttgagggtgtagaggtcctccacgcttgtttgcctgaaagtaatataatgatgctgtctgaacaggttttactgcttgctttccaagtaaaggttaattatgataataaagagatttggaactgcgccggcgccgccgggaagcgctaggcctggtccctcttcctaggatagcgttgcgcgcatgcgcctttgacgagtgagccggggagccatggacaactgtttggcggccgcagcgctgaatggggtggaccgacgttccctgcagcgttcagcaaagctggctctagaagtgctggagagggccaagaggagggcggtggactggcatgccctggagcgtcccaaaggctgcatgggggtccttgcccgggaggcgccccacctagagaaacagccggcagccggcccgcagcgcgttctcccgggagagaaatattattcatctgtgccagaggaaggaggggcaacccatgtctatcgttatcacagaggcgagtcgaagctgcacatgtgcttggacatagggaatggtcaggctgagaacatctctaaggacctctacatagaagtatatccagggacctattctgtcactgtgggctcaaatgacttaaccaagaagactcatgtggtagcagttgattctggacaaagcgtggacctggtcttccctgtgtgatgttgaccatcactgccatcacatcacctttttttaagtagtaagaataaagccactgtatgattctcttaatagctatacattaatcctgtttttagtgctgactgggtcagccttccgggaactggagtctgtctctttcagtgcttttttgtttgtttggttggttgttttttgagacagtctcactctgttgcccaggctggagtgcagtggcgtgatctcggctcactgcaagttccgcctcccgggttcacaccattctcctgcctcagcctcccgagtagctggcactacaggcacccgccaccatgcccggctattttttttgtatttttagtagagacggggtttcaccatgttggccaggatggtctcgatctcttgacctcgtgatccacccaccttggcctcccaaagtgttgggattacaggcgtgagccaccgcgcccggcctcagtgccttttttaacttgagggtgtagaggtcctccacgcttgtttgcctgaaagtaatataatgatgctgtctgaacaggttttactgcttgctttccaagtaaaggttaattatgataataaagagatttggaa

SEQ ID NO: 21 - protein, 156 aa, Homo sapiens
MDNCLAAAALNGVDRRSLQRSAKLALEVLERAKRRAVDWHALERPKGCMGVLAREAPHLEKQPAAGPQRVLPGEKYYSSVPEEGGATHVYRYHRGESKLHMCLDIGNGQAENISKDLYIEVYPGTYSVTVGSNDLTKKTHVVAVDSGQSVDLVFPV

SEQ ID NO: 22 - protein, 5 aa, Artificial
  Description of Artificial Sequence Hypothetical used as example
GGPGG

SEQ ID NO: 23 - protein, 5 aa, Artificial
  Description of Artificial Sequence Hypothetical used as example
GPPGG

SEQ ID NO: 24 - protein, 16 aa, Homo sapiens
CMEPVDAALLSSYETN

SEQ ID NO: 25 - protein, 14 aa, Homo sapiens
NRSNDSQLNDRWTF

SEQ ID NO: 26 - protein, 14 aa, Homo sapiens
AVDSGQSVDLVFPV

SEQ ID NO: 27 - protein, 41 aa, Artificial
  Description of Artificial Sequence Motif
CXXCXXXXXXXXXXXXXXCXHXXHXXCXXXXXXXXXXCXXC

SEQ ID NO: 28 - DNA, 15 bp, Homo sapiens
ccgccgccgccatat

SEQ ID NO: 29 - DNA, 123 bp, Homo sapiens
tgtgtgatctgtatgatggactttgtttatggggacccaattcgatttctgccgtgcatgcacatctatcacctggactgtatagatgactggttgatgagatccttcacgtgcccctcctgc

SEQ ID NO: 30 - DNA, 19 bp, Homo sapiens
atggacaactgtttggcga

SEQ ID NO: 31 - DNA, 15 bp, Homo sapiens
aaccctggggactat

SEQ ID NO: 32 - DNA, 300 bp, Homo sapiens
ccaaatgcctcttatccctgaattcagagtgataattttataagtgtgaaacttaattatgtagggctccccccgtctgaatagaattaattccttaaagtctagttagggtcctgctgtctgtcatgttgccttgtaacggatgtttccacctccttctccaacctctaccccaccattagtgtattttactataaaaacagtggaaccacagccctaaagtcctgctgatataaagtccttttgtcttaattgtatttaaaaaaaaaaaaaaaactactcttgatcacattagctatg

SEQ ID NO: 33 - DNA, 12 bp, Homo sapiens
tattattcatct

SEQ ID NO: 34 - DNA, 12 bp, Homo sapiens
tatcacagaggc

SEQ ID NO: 35 - DNA, 12 bp, Homo sapiens
tacatagaagta

SEQ ID NO: 36 - DNA, 12 bp, Homo sapiens
tatccagggacc

SEQ ID NO: 37 - DNA, 12 bp, Homo sapiens
tattctgtcact

SEQ ID NO: 38 - protein, 4 aa, Artificial
  Description of Artificial Sequence - SH2 domain site
YYSS

SEQ ID NO: 39 - protein, 4 aa, Artificial
  Description of Artificial Sequence - SH2 domain site
YSSV

SEQ ID NO: 40 - protein, 4 aa, Artificial
  Description of Artificial Sequence - SH2 domain site
YHRG

SEQ ID NO: 41 - protein, 4 aa, Artificial
  Description of Artificial Sequence - SH2 domain site
YIEV

SEQ ID NO: 42 - protein, 4 aa, Artificial
  Description of Artificial Sequence - SH2 domain site
YPGT

SEQ ID NO: 43 - protein, 4 aa, Artificial
  Description of Artificial Sequence - SH2 domain site
YSVT

SEQ ID NO: 44 - protein, 9 aa, Artificial
  Description of Artificial Sequence - tyrosine phosphorylation site
RTMAEFMDY

SEQ ID NO: 45 - DNA, 20 bp, Homo sapiens
agggaagaccaggtccacgc

SEQ ID NO: 46 - DNA, 15 bp, Homo sapiens
aaccctggggactat

(19) United States
(12) Patent Application Publication
(10) Pub. No.: US 2005/0065333 A1
(43) Pub. Date: Mar. 24, 2005

(54) Breast cancer-associated genes and uses thereof

(76) Inventor: Arun Seth, Mississauga (CA)

Correspondence Address: JONES DAY, 222 EAST 41ST ST, NEW YORK, NY, 10017, US

(21) Appl. No.: 10/476,362

(22) PCT Filed: Apr. 29, 2002
(86) PCT No.: PCT/US02/13584

Related U.S. Application Data
(60) Provisional application No. 60/287,170, filed on Apr. 27, 2001.

(51) Int. Cl. C12Q 1/68; G01N 33/574; C07H 21/04; C07K 14/72

(52) U.S. Cl. 536/23.5; 435/6; 435/7.23; 435/69.1; 435/320.1; 435/325; 530/350

(57) ABSTRACT

The present invention relates to seven isolated breast cancer-associated (BCA) polynucleotides, polypeptides, and variants thereof. The invention also relates to BCA antagonists. The invention also encompasses pharmaceutical compositions comprising BCA polynucleotides, BCA polypeptides, or BCA antagonists. The invention also contemplates methods for preventing or treating cancer, particularly breast cancer, comprising administering to a patient in need of such treatment a composition comprising a BCA polynucleotide, polypeptide, antagonist, or variant thereof. The invention also relates to methods for diagnosing, staging or determining a prognosis of a BCA-related disorder.

This application claims priority to U.S. provisional application Ser. No. 60/287,170, filed Apr. 27, 2001, which is incorporated herein by reference in its entirety.

### 1. INTRODUCTION

The present invention relates to seven isolated breast cancer-associated (BCA) polynucleotides, polypeptides, and variants thereof. The invention also relates to BCA antagonists. The invention also encompasses pharmaceutical compositions comprising BCA polynucleotides, BCA polypeptides, or BCA antagonists. The invention also contemplates methods for preventing or treating cancer, particularly breast cancer, comprising administering to a patient in need of such treatment a composition comprising a BCA polynucleotide, polypeptide, antagonist, or variant thereof. Further, invention also relates to methods for diagnosing, staging or determining a prognosis of a BCA-related disorder.

### 2. BACKGROUND

Breast cancer is the most common neoplastic disease in females, accounting for up to one third of all new cases of cancer in North American women. Breast cancer is the second leading cause of cancer-related deaths of women in the United States. About 180,000 new cases of breast cancer are diagnosed each year, a quarter of them fatal. Early detection is the key to the survival of these patients. However, there are no molecular markers to detect breast cancer at very early stages.

Progress in finding a cure for breast cancer will be aided by identifying specific etiologic agents, ascertaining the precise time of initiation, and uncovering the molecular mechanisms responsible for breast cancer initiation and progression. Despite the uncertainties surrounding the origin of breast cancer, breast cancer risk appears to be related to endocrinologic and reproductive factors. The development of breast cancer usually depends on endocrine conditions, such as early menarche, late menopause, and parity. However, the specific hormone or hormone combinations responsible for cancer initiation have not been identified, and their role as protective or risk factors remains incompletely understood. Estrogen is involved in the development of a variety of cancers, but it is still unclear whether estrogens are carcinogenic to the human breast.

Epidemiologic studies have shown that a family history of breast cancer increases a woman's risk of breast cancer when compared with the general population. Familial breast cancer is characterized by early onset and bilateral tumors, and also, in some cases, associated malignancies, most commonly ovarian cancer. Clinical studies suggest that breast cancer may be inherited as an autosomal dominant disease involving rare genes in which carriers have a high probability of developing the disease, perhaps as great as 100% in some families. It is estimated that 5-10% of all breast cancer cases are due to autosomal dominant genes segregating with the disease.

Several genes are known to correlate with inherited susceptibility to breast cancer, such as the ataxia-telangiectasia (AT) gene (11q22-q23), TP53 gene (17p13.1), androgen receptor (Xq11.2-q12), BRCA1 gene (17q21), and BRCA2 gene (13q12-q13). These genes differ dramatically, however, in terms of the breast cancer risk that these genes confer, the proportion of breast cancer incidences that express these genes, as well as other cancers with which these genes are associated.

Germline mutations in the TP53 gene are responsible for a high proportion of LI-Fraumeni families, in which breast cancer occurs in association with childhood sarcomas and other cancers. In such families, the risk of breast cancer is over 50% by age 50, and the risk of some type of cancer is nearly 100%. Germline TP53 mutations are probably responsible for less than one percent of all breast cancer, however (Easton et al., 1993, Cancer Surv. 18:95-113).

By contrast, heterozygotes for the AT gene exhibit a moderate risk of breast cancer, but because this gene is more common in the population, the AT gene may account greater than 7% of breast cancer cases (Easton et al., 1993, Cancer Surv 18:95-113).

Germline mutations in the androgen receptor are known to cause male breast cancer, but this has only been demonstrated in two families.

Genetic linkage studies have shown that some high risk breast cancer families, particularly those where breast cancer occurs in association with ovarian cancer, involve the BRCA1 gene (Easton et al., 1993, Cancer Surv 18:95-113). The BRCA1 gene confers a breast cancer risk of about 70% by age 70, but accounts for only for approximately 2% of breast cancer cases. Mutations in BRCA1 result in truncation and presumed inactivation of the BRCA1 protein. Analysis of BRCA1 function have indicated that it may act as an inhibitor of cell proliferation and, at least in mice, is necessary for normal development. The presence of a motif in BRCA1 characteristic of a family of proteins known as granins, has led to the suggestion that the protein is secreted into the extracellular space. BRCA1 is infrequently somatically mutated in sporadic breast or ovarian cancer.

The BRCA2 gene has recently been identified (Stratton, 1996, Hum Mol Genet 5:1515-9). The BRCA2 gene carries a risk of breast cancer similar to that of BRCA1, but is associated with a lower risk of ovarian cancer and a higher risk of male breast cancer. In addition to breast and ovarian cancer, germline BRCA2 mutations probably confer a small risk of a wide range of cancers. Somatic mutations of BRCA2 in sporadic breast and ovarian cancer are very rare.

The prevalence and penetrance of mutations in BRCA1 and BRCA2 have been studied extensively, yet only a small proportion of breast cancer is due to mutations in these genes. Phenotypic expression and penetrance of the known mutations in BRCA1 and BRCA2 is not currently predictable. Since the epidemiologic and genetic data suggest that breast cancer is a heterogeneous disease, it is likely that other genes play a role in breast cancer.

Evidence for additional high penetrance genes exists, but an understanding of multiple lower penetrance alleles will be necessary to fully define breast cancer risk. Multiple approaches are being used to identify additional high and low penetrance genes.

One approach has focused on the analysis of polymorphisms of potential functional significance in several classes of genes, including those involved in carcinogen metabolism, estrogen metabolite biosynthesis, steroid hormone receptor activation and DNA repair. For example, families with three or more breast cancer cases are being used in traditional linkage studies, which are expected to yield only moderate or high penetrance susceptibility genes. Breast cancer case-control studies are being used to look for genetic variants or polymorphisms that confer an increased risk of breast cancer in a wide variety of cellular pathways, ranging from the detoxification of environmental carcinogens to steroid hormone metabolism, DNA damage repair and immune surveillance, an approach useful primarily to identify low penetrance susceptibility genes. However, this approach has failed to produce convincing results to date.

Another approach, using BRCA1 and BRCA2 mutation carriers to identify genes that are associated with modification of breast cancer risk, has met with limited success. Clearly, breast cancer susceptibility is a complex phenomenon, in which multiple genes are likely involved. Thus, there remains a great need to explore different approaches to identify breast cancer-related genes. To this end, a greater understanding of the molecular changes in the development and maintenance of breast cancer would greatly facilitate efforts to detect and cure breast cancer.

### 3. SUMMARY OF THE INVENTION

The invention relates to isolated breast cancer-associated (BCA) polynucleotides, polypeptides, and antagonists (e.g., antibodies directed to BCA polypeptides), and their uses for drug screening. The invention also encompasses uses of BCA polynucleotides, polypeptides, and antagonists for the prevention, diagnosis, prognosis and management of cancer, particularly hormone-sensitive cancer, more particularly breast cancer.

The present invention is based, at least in part, on the discovery of cDNA molecules which encode BCA proteins that are differentially expressed in breast cancer cells as compared to normal breast cells.

BCA proteins and variants thereof of the present invention are collectively referred to as “polypeptides” or “proteins” of the invention. Nucleic acid molecules encoding the polypeptides or proteins of the invention, or their complements thereof (i.e., antisense polynucleotide), are collectively referred to as “polynucleotides” or “nucleic acid sequences” of the invention.

The present invention provides isolated polynucleotides encoding a polypeptide of the invention. The invention further provides isolated polynucleotides, or variants thereof, which can be used, for example, as hybridization probes or primers to detect or amplify nucleic acids encoding a polypeptide of the invention.

The present invention also provides isolated polynucleotides, or variants thereof, that can be used, for example, to screen for DNA-binding proteins, including but not limited to proteins that affect DNA conformation or modulate transcriptional activity (e.g., enhancers, transcription factors). In another embodiment, such probes can be used to screen for RNA-binding factors, including but not limited to proteins, steroid hormones, or other small molecules. In yet another embodiment, such probes can be used to detect and identify molecules that bind or affect the pharmacokinetics or activity (e.g., enzymatic activity) of a polypeptide of the invention.

The present invention also encompasses DNA vectors that comprise polynucleotide of the invention. In a further embodiment, the polynucleotides of the invention are operatively associated with a regulatory element that directs the expression of the polynucleotide. The invention also encompasses genetically engineered host cells that comprise any of the polynucleotides of the invention, operatively associated with a regulatory element that directs the expression of the polynucleotide in the host cell. Regulatory elements include, but are not limited to, inducible and non-inducible promoters, enhancers, operators and other elements that drive or regulate expression, which are known to the skilled artisan.

The present invention provides compositions comprising agonists or antagonists of a BCA polynucleotide, BCA polypeptide or complexes comprising a BCA polynucleotide or BCA polypeptide. Compositions comprising inhibitors of such agonists and antagonists are also encompassed by the present invention.

The present invention further provides methods for identifying such agonists, antagonists, or corresponding inhibitors. Such agonists, antagonists, or inhibitors can be small molecules (i.e., less than 500 daltons) that bind a BCA polynucleotide or BCA polypeptide of the invention.

Accordingly, the present invention relates to a composition comprising a BCA nucleic acid, BCA polypeptide, BCA agonist, BCA antagonist, inhibitor of a BCA agonist, inhibitor of a BCA antagonist, and/or a variant thereof. The present invention also encompasses a composition comprising a BCA nucleic acid, BCA polypeptide, BCA agonist, BCA antagonist,inhibitor of a BCA agonist, inhibitor of a BCA antagonist, and/or a variant thereof; and another therapeutic agent.

The present invention also relates to a method for preventing or treating a BCA-related disorder (e.g., breast cancer), comprising administering to a patient in need thereof an effective amount of a BCA nucleic acid, BCA polypeptide, BCA agonist, BCA antagonist, inhibitor of a BCA agonist, inhibitor of a BCA antagonist, and/or a variant thereof.

The present invention also relates to a method for preventing or treating a BCA-related disorder (e.g., breast cancer), comprising administering to a patient in need thereof an effective amount of a BCA nucleic acid, BCA polypeptide, BCA agonist, BCA antagonist, inhibitor of a BCA agonist, inhibitor of a BCA antagonist, and/or a variant thereof; and another therapeutic agent.

The present invention also relates to a method for increasing a patient's sensitivity to a therapeutic agent, comprising administering to a patient in need thereof an effective amount of a BCA nucleic acid, BCA polypeptide, BCA agonist, BCA antagonist, inhibitor of a BCA agonist, inhibitor of a BCA antagonist, and/or a variant thereof.

The present invention also relates to a method for diagnosing, staging or determining a prognosis of a BCA-related disorder, comprising the step of determining a level of BCA nucleic acid or BCA polypeptide expression in a biological tissue.

Accordingly, the present invention provides compositions and methods for the use of a BCA agonist or antagonist to prevent or treat a BCA-related disorder, such as cancer, in particular breast cancer.

BCA agonists include, but are not limited to, small molecules that bind a BCA polypeptide, antibodies directed to a BCA polypeptide, and other compounds that interact with a BCA polypeptide or a BCA gene to enhance its activity or expression.

BCA antagonists include, but are not limited to, antibodies to BCA polypeptides, BCA antisense oligonucleotides, BCA ribozymes, BCA triple-helix molecules, molecules that inhibit binding of regulatory proteins to regulatory regions of a BCA gene or otherwise inhibit BCA expression, and other small molecules that bind a BCA polypeptide, or otherwise inhibit BCA gene product activity.

The present invention also provides pharmaceutical compositions comprising a BCA nucleic acid, BCA polypeptide, BCA agonist, BCA antagonist, inhibitor of a BCA agonist, inhibitor of a BCA antagonist, and/or a variant thereof; and a pharmaceutically acceptable carrier. The present invention also encompasses pharmaceutical compositions comprising a BCA nucleic acid, BCA polypeptide, BCA agonist, BCA antagonist, inhibitor of a BCA agonist, inhibitor of a BCA antagonist, and/or a variant thereof, another therapeutic agent; and a pharmaceutically acceptable carrier. In particular, the present invention provides pharmaceutical compositions comprising a BCA antagonist, as well as methods for prophylactic and therapeutic use of pharmaceutical compositions comprising a BCA antagonist.

The invention also provides for drug delivery means and therapeutic regimens for the pharmaceutical compositions of the invention. In one embodiment, the pharmaceutical compositions of the invention are delivered by gene therapy.

A BCA antagonist can also be used to prevent occurrence, recurrence, or stop progression of a BCA-related disorder. For example, aside from affecting diseased tissue, a BCA antagonist can affect normal tissues, which include tissues containing cells that normally express a BCA gene. Additionally, a BCA antagonist can affect normal tissues that, although not expressing a BCA gene, are compromised by diseased tissues. In a particular embodiment, a BCA antagonist directly acts on diseased tissue thereby protecting normal surrounding tissues that do or do not normally express a BCA gene.

In one embodiment, a BCA antagonist is administered, to a patient in need of such treatment, to prevent or treat cancer, wherein a BCA mRNA or protein is expressed at above-normal levels.

In another embodiment, a BCA antagonist is administered to a patient in need of such treatment, at a high dose to prevent or treat cancer.

In another embodiment, a BCA antagonist is administered, to a patient in need of such treatment, at a low or reduced dose to prevent or treat cancer.

In yet another embodiment, a BCA antagonist is administered, to a patient in need of such treatment, for a short treatment cycle to prevent or treat cancer.

The invention further encompasses use of a BCA antagonist in combination therapy to prevent or treat cancer. Such therapy includes the use of one or more different molecules; compounds or treatments that assist in the prevention or treatment of a disease. In a specific embodiment, the invention provides for a BCA antagonist that is administered to a human, in combination with one or more cancer therapeutic agents, to prevent or treat cancer. Such cancer therapeutics include one or more molecules, compounds or treatments that have anti-cancer activity. Examples of contemplated therapeutics include biologicals, chemicals, and therapeutic treatments (e.g., irradiation treatment).

Accordingly, in a specific embodiment, the present invention provides for preventing or treating cancer comprising administering, to a patient in need of such treatment, a pharmaceutical composition comprising a BCA antagonist, and one or more therapeutic agents. In one embodiment, the BCA antagonist potentiates the effect of additional therapeutic agents. In another embodiment, the BCA antagonist sensitizes the patient to subsequent administration of additional therapeutic agents. Such combination treatments can reduce the overall toxicity of a therapeutic regimen. For example, lower dosages, fewer administrations, and shorter treatment periods can demonstrate fewer side effects or improved efficacy as compared to most standard treatments, such as standard treatments for cancer.

In a particular embodiment, the invention provides for a BCA antagonist that is administered to a human, in combination with one of more cancer therapeutic agents at reduced doses, to prevent or treat cancer. Such treatments may involve high, standard, or low doses of one or more BCA antagonists, and treatment cycles may be of long or short duration. In a specific embodiment, the invention provides for a particularly high dose of a BCA antagonist that is administered to a human, in combination with one of more cancer therapeutic agents at reduced doses, for short treatment cycles to prevent or treat cancer.

Preferred embodiments of the invention encompass a method for preventing or treating breast cancer, said method comprising administering to a subject in need thereof an amount of a pharmaceutical composition comprising a BCA polynucleotide; a BCA polypeptide; or an antibody that immunospecifically binds to a BCA polypeptide, effective for preventing or treating said cancer, and a pharmaceutically acceptable carrier, wherein said polynucleotide or polypeptide is selected from the group consisting of a BCA1, BCA2, BCA3, BCA4, BCA5, BCA6, and BCA7 polynucleotide or polypeptide. Also included is a method for preventing or treating breast cancer, said method comprising administering to a subject in need thereof an amount of (a) an expression vector comprising a human BCA polynucleotide; or (b) an antisense BCA polynucleotide; effective for preventing or treating said cancer, wherein said polynucleotide is selected from the group consisting of BCA1, BCA2, BCA3, BCA4, BCA5, BCA6, and BCA7.

Also included is a method for diagnosing a BCA-related disorder in a subject comprising the steps of (a) contacting a BCA antibody with a sample, suspected of containing a BCA polypeptide, from said subject under conditions that allow said BCA antibody to bind said BCA polypeptide; and (b) detecting or measuring binding of said BCA antibody to said BCA polypeptide; wherein said BCA polypeptide is selected from the group consisting of BCA1, BCA2, BCA3, BCA4, BCA5, BCA6, and BCA7; and wherein said BCA-related disorder is determined to be present when the presence or amount of BCA polypeptide indicated by the detection or measurement of binding differs from a control value representing the amount of BCA polypeptide present in an analogous sample from a subject not having said BCA-related disorder.

Also encompassed is a method for staging a BCA-related disorder in a subject comprising the steps of (a) contacting a BCA antibody with a sample, suspected of containing a BCA polypeptide, from said subject under conditions that allow said BCA antibody to bind said BCA polypeptide; and (b) detecting or measuring binding of said BCA antibody to said BCA polypeptide; wherein said BCA polypeptide is selected from the group consisting of BCA1, BCA2, BCA3, BCA4, BCA5, BCA6, and BCA7; and wherein the stage of a BCA-related disorder in a subject is determined when the presence or amount of BCA polypeptide indicated by the detection or measurement of binding is compared with the amount of BCA polypeptide present in an analogous sample from a subject having a particular stage of a BCA-related disorder.

Other preferred embodiments of the invention involve uses in drug screening for example, a method for identifying an analyte that binds a BCA polypeptide comprising the steps of (a) contacting said BCA polypeptide with an analyte under conditions that allow said analyte to bind said BCA polypeptide; and (b) detecting binding of said BCA polypeptide to said analyte; wherein said BCA polypeptide is selected from the group consisting of a BCA1, BCA2, BCA3, BCA4, BCA5, BCA6, and BCA7 polypeptide.

Also included is a method for identifying a protein that binds a BCA polypeptide comprising the steps of (a) contacting said BCA polypeptide with a positionally addressable array comprising a plurality of proteins, with each protein being at a different position on a solid support; and (b) detecting binding of said BCA polypeptide to a protein on said array; wherein said BCA polypeptide is selected from the group consisting of a BCA1, BCA2, BCA3, BCA4, BCA5, BCA6, and BCA7 polypeptide.

Also included is a method for identifying an analyte that binds a complex comprising a BCA polynucleotide or BCA polypeptide comprising the steps of (a) contacting said complex with said analyte under conditions that allow said analyte to bind said complex; and (b) detecting binding of said BCA polynucleotide or BCA polypeptide to said analyte; wherein said analyte binds to said BCA polynucleotide or BCA polypeptide when bound to said binding partner, and does not bind to said BCA polynucleotide or BCA polypeptide when not bound to said binding partner.

Also encompassed is a method for identifying an analyte that inhibits formation of a complex comprising a BCA polynucleotide or BCA polypeptide comprising the steps of (a) contacting said complex with said analyte; and (b) measuring the amount of said complex; wherein a reduction in the amount of complex indicates that said analyte inhibits formation of said complex.

Also encompassed is a method for identifying an inhibitor of growth of a breast cancer cell comprising the steps of (a) contacting said cell with (i) a BCA polynucleotide; (ii) a BCA polypeptide; or (iii) an antibody that immunospecifically binds to a BCA polypeptide; (b) measuring cell growth; wherein said polynucleotide or polypeptide is selected from the group consisting of a BCA1, BCA2, BCA3, BCA4, BCA5, BCA6, and BCA7 polynucleotide or polypeptide; and wherein an inhibition of cell growth indicates the presence of an inhibitor of growth of a breast cancer cell.

### 3.1. Definitions

As used herein, the phrase “BCA nucleic acid” or “BCA polynucleotide” refers to a polynucleotide derived from one or more of the genes BCA1, BCA2, BCA3, BCA4, BCA5, BCA6 and BCA-7 (“BCA1-7”), including the complementary sequences thereof, and variants thereof.

As used herein, the phrase “BCA polypeptide” refers to a protein, polypeptide, peptide, and variants thereof, derived from the one or more of the genes or cDNAs (e.g., including polypeptides encoded by mRNA splice variants) of BCA1, BCA2, BCA3, BCA4, BCA5, BCA6 and BCA-7.

As used herein, the term “variant” or “variants” refers to, where appropriate, variations of the nucleic acid or amino acid sequence of BCA molecules such as, but not limited to, homologs, analogs, derivatives, fragments, hybrids, mimetics, congeners, and nucleotide and amino acid substitutions, additions, deletions, or other chemical modifications.

As used herein, the phrase “BCA-related disorder” refers to a disease that involves regulation of a BCA gene, and includes, but is not limited to, diseases involving cells expressing a BCA gene, particularly diseases involving above-normal or unregulated expression of a BCA gene. BCA-related disorders include, but are not limited to, cell proliferative disorders and pathologies of cells or tissues that are affected by cells that express a BCA gene or a BCA-related gene.

As used herein, the phrase “BCA gene expression” refers to transcription of a BCA gene which produces BCA pre-mRNA, BCA mRNA, and/or translation of BCA mRNA to produce BCA protein.

As used herein, the phrase “therapeutics” or “therapeutic agents” refer to any molecules or compounds that assist in the treatment of a disease. As such, a cancer therapeutic is a molecule or compound that aids in the treatment of tumors or cancer. A treatment protocol includes, but is not limited to, administration of therapeutic agents, radiation therapy, dietary therapy, physical therapy, and psychological therapy. Cancer therapeutics also encompass a molecule or compound that aids in the prevention of tumors or cancer, prevents the recurrence of tumors or cancer, or prevents the spread or metastasis of tumors or cancer.

As used herein, a “naturally-occurring” polynucleotide refers to an RNA or DNA molecule having a nucleotide sequence that occurs in nature (e.g., encodes a natural protein).

### 4. BRIEF DESCRIPTION OF THE FIGURES

FIGS. 1A and 1B. Coordinate expression of novel breast cancer-associated genes in primary tumors. The figure depicts autoradiographs of arrayed cDNAs from a breast cancer library after hybridization with complex probes derived from normal breast tissue (FIG. 1A) or breast tumors (1B) as indicated.

FIGS. 2A, 2B and 2C. Chromosomal localization of BCA genes by FISH mapping. The figure depicts the chromosomal mapping of three BCA genes by fluorescence in situ hybridization. Fluorescence in situ hybridization signals of genomic PAC clones obtained for three breast cancer associated cDNAs are shown. Band assignments were determined by measuring fractional chromosome length and analyzing the banding pattern generated by DAPI counterstained image. FIG. 2A. BCA1; FIG. 2B. BCA2; FIG. 2C. BCA3.

FIG. 3. Expression of breast cancer-associated genes in normal human tissues. The figure depicts a northern blot of human tissue (heart, brain, placenta, lung, liver, skeletal muscle, kidney, pancreas, spleen, thymus, prostate, testis, ovary, small intestine, colon and lymphocytes) probed with BCA1, BCA2, BCA3 and β-actin cDNAs.

FIG. 4A shows the expression of BCA1, BCA2, BCA3, BCA5, and BCA6 in breast carcinoma cell lines. Total RNA was extracted from BT483 (lane 1), HTB24 (lane 2), HTB26 (lane 3), HTB126 (lane 4), MCF-7 (lane 5) and MDA-MB-468 (lane 6) and northern blotted using ³²P-labeled full-length BCA1, BCA2, BCA3, BCA5 or BCA6 cDNA probes as indicated. The lower panel shows ethidium bromide staining of ribosomal RNA.

FIG. 4B shows the expression of BCA4 and BCA7 in ER− and ER+ breast carcinoma cell lines. Total RNA was extracted from ER− cells (MCF-7 with adriamycin (lane 1), MDA-MB453 (lane 2), MDA-MB231 (lane 3), and from ER+ cells ZR75-1 (lane 4), MCF-7 (lane 5), human mammary epithelial cells with benz(a)pyrene (lane 6), fibroadenoma cells (lane 7), and human mammary epithelial cells without (lanes 8-9) benz(a)pyrene, run on a 1% agarose gel, and probed with ³²P-labeled BCA4 or BCA7 cDNA. The lower panel shows ribosomal RNA after ethidium bromide staining.

FIG. 4C shows expression of BCA genes in human prostate and breast tumor cell lines. Autoradiograms of Northern blotted RNA from prostate tumor cell lines (LnCap, Du145, PC3) and breast tumor cell lines (MCF7, MDA MB 468, HTB126) hybridized with radiolabeled full-length BCA1, BCA2, or BCA3 cDNA probes.

FIGS. 5A and 5B. BCA1 cDNA sequence (FIG. 5A) (SEQ ID NO.: 1) and predicted amino acid sequence (FIG. 5B) (SEQ ID NO.: 2). FIG. 5A. Open reading frame encoding the BCA1 protein shown in uppercase and underlined letters. “PY” motif (nts 363-377) and RING-H2 domain (nts 551-674) underlined in bold. Initial clone sequence (nts 1556-2033) shown in bold. Splice junctions: exons I/II at nt 378, exons II/III at nt 550. FIG. 5B. The RING-H2 domain (aa 99-139) (CVICMMDFVYGDPIRFLPCMHIYHLDCIDDWLMRSFTCPSC (SEQ ID NO.: 15)) and “PY” motif (aa 36-40) (PPPPY (SEQ ID NO.: 16)) region are underlined in bold in the amino acid sequence. Splice junctions: I/II at aa/codon 41, exons II/III at aa/codon 98.

FIGS. 6A and 6B. BCA1 gene structure, mRNA, and open reading frame (ORF). FIG. 6A. Genomic and cDNA/mRNA organization. Sizes of exons and introns are indicated. Positions of the exon junction boundaries are indicated in accordance with the BCA1 cDNA sequence. FIG. 6B. BCA1 amino acid residues numbered above with PY motif and RING-H2 domain indicated by semitransparent boxes. Amino acid positions of the domains are also indicated (SEQ ID NO.: 2).

FIG. 7 indicates the protein binding and phosphorylation sites found in BCA1 including CK2 and PKC consensus sequences and SH2 domains (SEQ ID NO.: 2, shown twice).

FIG. 8 depicts immunostaining of BCA1 in invasive breast tumor. The HistoStain SP Kit (rabbit, Zymed, San Francisco, Calif.) was used for immunohistochemical analysis of BCA1 protein in paraffin-embedded breast tissues. Immunoperoxidase staining was as described (Soubeyran et al., 1995) with the modification that removal of endogenous peroxidase was followed by incubation in 0.1% trypsin (pH 9.10). BCA1 antibody (dil. 1:25, 40 μg/ml) was added for 2 h at room temperature, negative control sections were incubated with preimmune serum. The Elston-modified Bloom and Richardson method was used for histological grading of breast tumors (Elton and Ellis, 1991).

FIG. 9 depicts immunohistochemistry of breast tumor tissue. Immunostaining was performed as in FIG. 8. Proteins reactive to BCA1 peptide antisera appear brown.

FIG. 10 shows BCA1 protein expressed in bacteria. Bacteria transformed with pGEX-vector or pGEX-BCA1, were induced with IPTG. Bacterial lysates were immunoblotted with anti-BCA1 C-terminal peptide antibody and visualized by chemiluminescence.

FIGS. 11A and 11B depicts an autoradiogram (FIG. 11A) and an immunoblot (FIG. 11B) of BCA1 expressed from pCMV-BCA1. In FIG. 11A, pCMV-BCA1 was used for in vitro translation with the TnT rabbit reticulocyte system to produce a single protein band on an autoradiogram. An immunoblot (FIG. 11B) of this reaction was probed with anti-FLAG antibody and visualized by chemiluminescence.

FIGS. 12A and 12B. BCA2 cDNA sequence (FIG. 12A) (SEQ ID NO.: 3) and predicted amino acid sequence (FIG. 12B)(SEQ ID NO.: 4). FIG. 12A. Open reading frame encoding the BCA2 protein shown in uppercase letters. NPxxY motif (nts 768-782) and RING-H2 domain (nts 980-1052) underlined in bold. Initial clone sequence (nts 1376-1644) shown in bold. Splice junctions: exons I/II at nt 349, exons II/III at nt 407, exons III/IV at nt 466, exons IV/V at nt 676, exons V/VI at nt 747, exons VI/VII at nt 819, exons VII/VIII at nt 914, exons VIII/IX at nt 1030. FIG. 12B. The NPxxY motif (aa 174-178) (NPGDY) and RING-H2 domain (aa 228-268) (CPVCKEDYTVEEEVRQLPCNHFFHSSCIVPWLELHDTCPVC (SEQ ID NO.:18)) are underlined in bold in the amino acid sequence. Splice junctions: exons I/II at aa/codon 34, exons II/III at aa/codon 53, exons III/IV at aa/codon 73, exons IV/V at aa/codon 142, exons V/VI at aa/codon 167, exons VI/VII at aa/codon 191, exons VII/VIII at aa/codon 222, exons VIII/IX at aa/codon 261.

FIGS. 13A and 13B. BCA2 gene structure, mRNA, and open reading frame (ORF). FIG. 13A. Genomic and cDNA/mRNA organization. Sizes of exons and introns are indicated. Positions of the exon junction boundaries are indicated in accordance with the BCA2 cDNA sequence. FIG. 13B. BCA2 amino acid residues numbered above with consensus SH2-binding site and Ring-H2 domain indicated by semitransparent boxes. Amino acid positions of the domains are also indicated.

FIGS. 14A and 14B. BCA3 cDNA sequence (FIG. 14A) (SEQ ID NO.:5) and predicted amino acid sequence (FIG. 14B)(SEQ ID NO.:6). FIG. 14A. Open reading frame encoding the BCA3 protein shown in uppercase letters. Consensus SH2 domain binding sequences (nts 399-410; nts 447-458; three between nts 606-641) underlined in bold. Initial T5D6 clone sequence (nts 369-651) shown in bold. Splice junctions: exons I/II at nt 86, exons II/III at nt 314, exons III/IV at nt 395, exons IV/V at nt 502, exons V/VI at nt 581. FIG. 14B. Consensus SH2 domain binding sequences (aa 104-107; aa 119-123; three between aa 172-183) underlined in bold. Splice junctions: exons I/II at aa/codon −2, exons II/III at aa/codon 74, exons III/IV at aa/codon 101, exons IV/V at aa/codon 136, exons V/VI at aa/codon 163.

FIGS. 15A and 15B. BCA3 variant 1 (exon III deleted) cDNA sequence (FIG. 15A) (SEQ ID NO.:19) and predicted amino acid sequence (FIG. 15B)(SEQ ID NO.:20). FIG. 15A. Open reading frame encoding the BCA3 protein shown in uppercase letters. Consensus SH2 domain binding underlined in bold. Splice junctions: exons I/II at nt 86, exons II/IV at nt 314, exons IV/V at nt 419, exons V/VI at nt 500. FIG. 15B. Consensus SH2 domain binding sequences (aa 103-106; aa 119-122; three between aa 171-183) underlined in bold. Splice junctions: exons I/II at aa/codon −2, exons II/IV at aa/codon 74, exons IV/V at aa/codon 109, exons V/VI at aa/codon 136.

FIGS. 16A and 16B. BCA3 variant 2 (exons III and V deleted) cDNA sequence (FIG. 16A) (SEQ ID NO.:21) and predicted amino acid sequence (FIG. 16B) (SEQ ID NO.:22). FIG. 16A. Open reading frame encoding the BCA3 protein shown in uppercase letters. Consensus SH2 domain binding underlined in bold. Splice junctions: exons I/II at nt 86, exons II/IV at nt 314, exons IV/VI at nt 419. FIG. 16B. Consensus SH2 domain binding sequences underlined in bold. Splice junctions: exons I/II at aa/codon −2, exons II/IV at aa/codon 74, exons IV/VI at aa/codon 109.

FIG. 17 indicates the protein binding and phosphorylation sites found in BCA3 including protein kinase C (“PKC”)and casein kinase 2 (“CK2”) and PKC consensus sequences, and cAMP-regulated phosphorylation domains and SH2 domains (SEQ ID NO.:6, shown twice).

FIGS. 18A and 18B. BCA3 gene structure, mRNA, splice variants, and open reading frame (ORF). FIG. 18A. Genomic and cDNA/mRNA organization and variants derived form cDNAs of UniGene Cluster Hs.283807 (C11ORF17), T5D6 (GenBank Accession No. AW225339), and overlapping EST sequences. Sizes of exons and introns are indicated. Positions of the exon junction boundaries are indicated in accordance with the BCA2 cDNA sequence. Variants lacking exon 3 or exon 3 and exon 5 are also depicted. FIG. 18B. BCA3 amino acid residues numbered above with consensus SH2-binding sites indicated by semitransparent boxes. Amino acid positions of the domains are also indicated.

FIGS. 19A-F. FIG. 19A. Ethidium bromide-stained 1.2% agarose gel of DNA amplified by RT-PCR using BCA3 cDNA primers and RNA extracted from the HTB-26 tumor cell line. FIG. 19B. In vitro expression of BCA3 variants from plasmid pCMV-BCA3. The autoradiogram of ³⁵S labeled protein produced by the TnT rabbit reticulocyte expression system. Lane 1 shows pCMV-BCA3 full length (663 bp); lane 2 shows pCMV-BCA3 variant 2 (471 bp); lane 3 is pCMV-BCA3 variant 1 (552 bp); lane 4 shows pCMV vector without BCA3 cDNA. FIG. 19C. Immunoblot of lysates from 293T cells transfected with pCMV-BCA3 visualized by chemiluminescent detection of anti-FLAG antibody. FIG. 19D. Ethidium bromide-stained 2% agarose gel showing RT-PCR products using BCA3 cDNA primers and RNA extracted from tumor cell lines and controls as indicated. FIG. 19E. Autoradiogram of Northern blotted total RNA extracted from human tumor cell-lines and normal breast tissue hybridized with cDNA probes for BCA3. Lower panel shows ribosomal RNA after ethidium bromide staining. FIG. 19F. Northern blot showing BCA3 and GAPDH expression in prostate cancer cell lines as indicated.

FIG. 20 depicts the hydropathy profile of the protein encoded by BCA1 using the Kyte-Doolittle method of calculating hydrophilicity over a window length of 17.

FIG. 21 depicts the hydropathy profile of the protein encoded by BCA2 using the Kyte-Doolittle method of calculating hydrophilicity over a window length of 17.

FIG. 22 depicts the hydropathy profile of the protein encoded by BCA3 using the Kyte-Doolittle method of calculating hydrophilicity over a window length of 17.

### 5. DETAILED DESCRIPTION OF THE INVENTION

The present invention is based, at least in part, on the discovery of cDNA molecules that encode particular breast cancer-associated (BCA) proteins, which are more highly expressed in breast cancer cells than in normal breast cells. Accordingly, the present invention relates to isolated BCA polynucleotides derived from the genes BCA1, BCA2, BCA3, BCA4, BCA5, BCA6 or BCA7, and variants thereof. The invention also relates to polypeptides encoded by the polynucleotides of the invention. The invention also relates to agonists or antagonists (e.g., antibodies) of a BCA polynucleotide, BCA polypeptide or complexes comprising a BCA polynucleotide or BCA polypeptide. The invention also relates to methods of identifying agonists and antagonists of a BCA polynucleotide or polypeptide. Further, the invention relates to prevention or treatment of a BCA-related disorder (e.g., breast cancer) comprising administering a BCA polynucleotide, BCA polypeptide, BCA agonist, BCA antagonist with or without additional therapeutic agents.

A polynucleotide is intended to include DNA molecules (e.g., cDNA, genomic DNA), RNA molecules (e.g., hnRNA, pre-mRNA, mRNA), and DNA or RNA analogs generated using nucleotide analogs. The polynucleotide can be single-stranded or double-stranded.

An isolated polynucleotide is one which is distinguished from other polynucleotides that are present in the natural source of the polynucleotide. Preferably, an “isolated” polynucleotide lacks flanking sequences (i.e., sequences located at the 5′ and 3′ ends of the nucleic acid), which naturally flank the nucleic acid sequence in the genomic DNA of the organism from which the nucleic acid is derived. For example, in various embodiments, the isolated polynucleotide can comprise less than about 5 kb, 4 kb, 3 kb, 2 kb, 1 kb, 0.5 kb or 0.1 kb of nucleotide sequences which naturally flank the polynucleotide in genomic DNA of the cell from which the nucleic acid is derived. In other embodiments, the isolated polynucleotide is about 10-20, 21-50, 51-100, 101-200, 201-400, 401-750, 751-1000, 1001-1500 bases in length.

Moreover, an “isolated” polynucleotide, such as a cDNA molecule, can be substantially free of other cellular material, or culture medium when produced by recombinant techniques, or substantially free of chemical precursors or other chemicals when chemically synthesized. As used herein, an isolated polynucleotide does not include an isolated chromosome, and does not include the poly(A) tail of an mRNA, if present.

The DNA sequence of BCA1 is SEQ ID NO:1 and the amino acid sequence is SEQ ID NO:2. The DNA sequence of BCA2 is SEQ ID NO:3 and the amino acid sequence is SEQ ID NO:4. The DNA sequence of BCA3 is SEQ ID NO:5 and the amino acid sequence is SEQ ID NO:6. The DNA sequence of BCA4 is SEQ ID NO:7 and the amino acid sequence is SEQ ID NO:8. The DNA sequence of BCA5 is SEQ ID NO:9 and the amino acid sequence is SEQ ID NO:10. The DNA sequence of BCA6 is SEQ ID NO:11 and the amino acid sequence is SEQ ID NO:12. The DNA sequence of BCA7 is SEQ ID NO:13 and the amino acid sequence is SEQ ID NO:14.

The present invention provides isolated polynucleotides encoding a BCA polypeptide and variants thereof. An isolated polynucleotide that encodes a variant polypeptide can be created by introducing one or more nucleotide substitutions, additions or deletions into the nucleotide sequence of SEQ ID NO:1, SEQ ID NO:3 (or GenBank Accession No. AW225336), SEQ ID NO:5 (or GenBank Accession No. AW225339), SEQ ID NO:7 (or GenBank Accession No. AW225341), SEQ ID NO:9 (or GenBank Accession No. AW225337), SEQ ID NO: 11 (or GenBank Accession No. AW225338), or SEQ ID NO:13 (or GenBank Accession No. AW225340) using any method known in the art. In one embodiment, such methods introduce one or more amino acid substitutions, additions or deletions are introduced into the encoded polypeptide.

For example, mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid, asparagine, glutamine), uncharged polar side chains (e.g., glycine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

Also encompassed by the present invention are specific portions of a BCA polynucleotide and the polypeptide encoded by such portions. For example, individual subclones or subsequences used to assemble the full-length (or nearly full-length) BCA cDNA or gene (“BCA subsequences”) are encompassed by the BCA polynucleotides of the invention. Accordingly, any polypeptide encoded by such subclones or subsequences is encompassed by the BCA polypeptides of the invention.

In addition, polynucleotides comprising partially assembled subsequences, or hybrid molecules comprising BCA subsequences or partially assembled subsequences, are encompassed by the polynucleotides of the invention. Accordingly, any polypeptide encoded by such hybrid molecules or partially assembled subsequences is encompassed by the BCA polypeptides of the invention.

Further, the present invention encompasses specific portions of a BCA polynucleotide or BCA polypeptide that can be discerned as a domain or motif such as, for example, a portion of a BCA polynucleotide or polypeptide having a predicted biological activity. Such domains and motifs include, but are not limited to, ring domains, PPPPY motifs, SH2 binding motifs, NPxxY motifs, zinc finger domains, phosphorylation sites, exons, introns, splice acceptor sites, splice donor sites, 5′ regulatory regions of the mRNA, 3′ regulatory regions of the mRNA, mRNA capping regions, promoter regions, transcriptional regulatory sites, enhancer sequences, glycosylation sites, ligand-binding sites, and variants thereof. Accordingly, a polynucleotide encoding such motifs or domains is encompassed by the BCA polynucleotides of the invention, and any polypeptide encoded by such BCA polynucleotides is encompassed by the BCA polypeptides of the invention.

Accordingly, a BCA polynucleotide can comprise cDNA, genomic DNA, introns, exons, promoter regions, 5′ regulatory regions of the gene, 3′ regulatory regions of the gene, RNA, hnRNA, mRNA, regulatory regions within RNAs, and variants thereof.

Motifs and domains can be identified using methods well known in the art such as, for example, computer software packages for molecular biological studies. Similarly, assays well known in the art for determining and/or measuring biological activity can be used to identify BCA polynucleotides or polypeptides exhibiting such activity. For example, promoter sequences for BCA1-7 can be determined by promoter-reporter gene assays and in vitro binding assays.

Using all or a portion of the nucleic acid sequences of SEQ ID NO:1, SEQ ID NO:3 (or GenBank Accession No. AW225336), SEQ ID NO:5 (or GenBank Accession No. AW225339), SEQ ID NO:7 (or GenBank Accession No. AW225341), SEQ ID NO:9 (or GenBank Accession No. AW225337), SEQ ID NO:11 (or GenBank Accession No. AW225338), or SEQ ID NO:13 (or GenBank Accession No. AW225340), as a hybridization probe, polynucleotides of the invention can be isolated using standard hybridization and cloning techniques (See, e.g., Sambrook et al., Molecular Cloning: A Laboratory Manual, 2nd ed., Cold Spring Harbor Laboratory, Cold Spring Harbor Laboratory Press, Cold Spring Harbor, N.Y., 1989).

In particular embodiments, a BCA polynucleotide comprises the BCA1 exon I (251 bp), BCA1 exon II (166 bp), BCA1 exon III (2106 bp), or intervening introns of approximately 33,073 bases and 1024 bases of the BCA1 gene, or variants thereof. The invention also encompasses an isolated polynucleotide comprising a nucleotide sequence of at least 12 consecutive bases encoding a portion of a domain of a human BCA1 polynucleotide or polypeptide, wherein said domain is selected from the group consisting of a RING H2 finger, PY motif, glycosylation site, phosphorylation site, SH2-binding motif, open-reading frame, exon 1, exon 2, exon 3, intron 1, intron 2, 5′ untranslated region, and 3′ untranslated region.

In other embodiments, a BCA polynucleotide comprises the BCA2 exon I (351 bp), BCA2 exon II (60 bp), BCA2 exon III (60 bp), BCA2 exon IV (211 bp), BCA2 exon V (74 bp), BCA2 exon VI (74 bp), BCA2 exon VII (96 bp), BCA2 exon VIII (117 bp), e BCA2 exon IX (728 bp), and intervening introns of approximately 64, 857, 4307, 12615, 18354, 1478, 935, 2296, and 996 bases of the BCA2 gene, or variants thereof. The invention also encompasses an isolated polynucleotide comprising a nucleotide sequence of at least 12 consecutive bases encoding a portion of a domain of a human BCA2 polynucleotide or polypeptide, wherein said domain is selected from the group consisting of a RING H2, NPXXY motif, PXXP motif, zinc finger, glycosylation site, phosphorylation site, SH3-binding motif, open-reading frame, exon 1, exon 2, exon 3, exon 4, exon 5, exon 6, exon 7, exon 8, exon 9, intron 1, intron 2, intron 3, intron 4, intron 5, intron 6, intron 7, intron 8, 5′ untranslated region, and 3′ untranslated region.

In other embodiments, a BCA polynucleotide comprises the BCA3 exon I (84 bp), BCA3 exon II (228 bp), BCA3 exon III (81 bp), BCA3 exon IV (105 bp), BCA3 exon V (81 bp), BCA3 exon VI (217 bp) and BCA3 exon VII (884 bp) and intervening introns of approximately 220, 779, 2294, 1969, 2355 and 29 bases of the BCA3 gene, or variants thereof. The invention also encompasses an isolated polynucleotide comprising a nucleotide sequence of at least 12 consecutive bases encoding a portion of a domain of a human BCA3 polynucleotide or polypeptide, wherein said domain is selected from the group consisting of a SH2 site YYSS, SH2 site YSSV, SH2 site YHRG, SH2 site YIEV, SH2 site YPGT, SH2 site YSVT, tyrosine phosphorylation site, RTMAEFMDY, glycosylation site, phosphorylation site, tyrosine phosphorylation motif, SH2-binding motif, open-reading frame, open-reading frame lacking exon 3, open-reading frame lacking exon 3 and exon 5, exon 1, exon 2, exon 3, exon 4, exon 5, exon 6, exon 7, intron 1, intron 2, intron 3, intron 4, intron 5, intron 6, 5′ untranslated region, and 3′ untranslated region.

In other embodiments, the above BCA polynucleotides comprise the 5′ half, 3′ half, 5′ third, middle third, 3′ third, first quarter (5′ most), second quarter, third quarter or fourth quarter (3′ most). For example, the invention encompasses an isolated polynucleotide comprising a BCA1 nucleotide sequence, wherein said sequence is selected from the group consisting of residues 1-2659, 1-2500, 1-2000, 1-1500, 1-1000, 1-500, 1-124, 2516-2659, 2500-2659, 2000-2659, 1500-2659, 1000-2659, 500-2659, 124-2659, 363-377, 551-674 of SEQ ID NO: 1, 5′-CCGCCGCCGCCATAT-3′ (SEQ ID NO: 29), and 5′-TGTGTGATCTGTATGATGGACTTTGTTTATGGGGACCCAATTCGATTTCTGCCGTGCATGCACATCTATCACCTGGACTGTATAGATGACTGGTTGATGAGATCCTTCACGTGCCCCTCCTGC-3′ (SEQ ID NO: 30). The invention also encompasses an isolated polynucleotide comprising a BCA2 nucleotide sequence, wherein said sequence is selected from the group consisting of residues 1-2176, 1-2000, 1-1500, 1-1000, 1-500, 1-100, 2000-2176, 1500-2176, 1000-2176, 500-2176, 100-2176, 768-782, 980-1052 of SEQ ID NO: 3, 5′-ATGGACAACTGTTTGGCGA-3′ (SEQ ID NO.:31), AGGGAAGACCAGGTCCACGC-3′ (SEQ ID NO: 46), 5′-AACCCTGGGGACTAT-3′ (SEQ ID NO: 47), 5′-AACCCTGGGGACTAT-3′ (SEQ ID NO.:32) and 5′-CCAAATGCCTCTTATCCCTGAATTCAGAGTGATAATTTTATAAGTGTGAAACTTAATTATGTAGGGCTCCCCCCGTCTGAATAGAATTAATTCCTTAAAGTCTAGTTAGGGTCCTGCTGTCTGTCATGTTGCCTTGTAACGGATGTTTCCACCTCCTTCTCCAACCTCTACCCCACCATTAGTGTATTTTACTATAAAAACAGTGGAACCACAGCCCTAAAGTCCTGCTGATATAAAGTCCTTTTGTCTTAATTGTATTTAAAAAAAAAAAAAAAACTACTCTTGATCACATTAGCTATG-3′ (SEQ ID NO.:33). The invention also encompasses An isolated polynucleotide comprising a BCA3 nucleotide sequence, wherein said sequence is selected from the group consisting of residues 1-1756, 1-1686, 1-1500, 1-1000, 1-500, 1-100, 1686-1756, 1500-1756, 1000-1756, 500-1756, 100-1756, 399-410, 446-457, 606-617, 618-629, 630-641 of SEQ ID NO: 5, 5′-TATTATTCATCT-3′ (SEQ ID NO.:34), 5′-TATCACAGAGGC-3′ (SEQ ID NO.:35), 5′-TACATAGAAGTA-3′ (SEQ ID NO.:36), 5′-TATCCAGGGACC-3′ (SEQ ID NO.:37), and 5′-TATTCTGTCACT-3′ (SEQ ID NO.:38).

Further, a BCA polynucleotide can comprise two or more of any above-described sequences, or variants thereof, linked together to form a larger subsequence.

Subsequences can also comprise polynucleotides of expressed sequence tags (“ESTs”) used to assemble a larger sequence, or ESTs overlapping or embedded within any BCA polynucleotide such as, but not limited to, the above-recited exons and introns.

In certain embodiments, the BCA polynucleotides and polypeptides specifically proviso out sequences consisting of those ESTs and species homologs known in the art. For example, in one embodiment, a BCA1 polynucleotide excludes a sequence consisting of the DNA sequence of the pufferfish genomic clone, AF022814. In another embodiment, a BCA3 polynucleotide comprises at least fourteen bases, wherein the polynucleotide is not a known EST which includes F29989, BG754249, BG654786, AU146189, AU145473, AV729000, AV725974, BE349302, BE205860, AW406755, AW339687, AI635272, AI365988, BM469324, BM558580, AW510839, BF337353, AA640772, AL599210, AL571890, BF913170, BE149796, BG681808, AA478355, BE304890, BI058894, BM042507, BG773327, AA521399, AA521323, AI873852, BI030630, BI023028, BG819532, BE909262, BE293845, BE293802, AW675725, AW193295, F19258, AI358229, AA478297, BG566176, AJ400877, NM_{—}020642, and BM449949.

The present invention also encompasses, in addition to the polynucleotides disclosed herein, (1) any nucleic acid that encodes a BCA polypeptide of the invention; (2) the complement of any nucleic acid that encodes a BCA polypeptide of the invention; (3) any polynucleotide that hybridizes to the complement of the sequences disclosed herein under highly stringent conditions, e.g., washing in 0.1×SSC/0.1% SDS at 68° C. (Ausubel et al., Current Protocols in Molecular Biology, Vol. I, Green Publishing Associates, Inc., and John Wiley & Sons, Inc., New York (1989) p. 2.10.3); and/or (4) any polynucleotide that hybridizes to the complement of the sequences disclosed herein under less stringent conditions, such as moderately stringent conditions, e.g., washing in 0.2×SSC/0.1% SDS at 45° C. (Ausubel et al., 1989, supra). In further embodiments, the polynucleotides encodes a functionally equivalent gene product.

In one embodiment, a variant BCA polynucleotide hybridizes to a naturally-occurring BCA polynucleotide under stringent conditions. In another embodiment, a variant BCA polynucleotide hybridizes to a naturally-occurring BCA polynucleotide under moderately stringent conditions.

The term “hybridizes under highly stringent conditions” is intended to describe conditions for hybridization and washing under which nucleotide sequences that are at least 60%, 65%, 70%, or preferably 75% identical to each other typically remain hybridized to each other. Such stringent conditions are known to those skilled in the art and can be found in Current Protocols in Molecular Biology, John Wiley & Sons, N.Y. (1989) pp. 6.3.1-6.3.6. A preferred, non-limiting example of stringent hybridization conditions are hybridization in 6× sodium chloride/sodium citrate (“SSC”) at about 45° C. followed by one or more washes in 0.2×SSC, 0.1% SDS at 50-65° C. Preferably, an isolated polynucleotide of the invention that hybridizes under stringent conditions to the sequence of SEQ ID NO: 1, 3 (or GenBank Accession No. AW225336), 5 (or GenBank Accession No. AW225339), 7 (or GenBank Accession No. AW225341), 9 (or GenBank Accession No. AW225337), 11 (or GenBank Accession No. AW225337), or 13 (or GenBank Accession No. AW225340}, or a complement thereof.

The present invention also encompasses polynucleotide variants that are revealed from inter-species comparisons of homologs of the BCA1-7 polynucleotides. As such, homologs of a BCA polynucleotide of the invention that are found in other species are encompassed by the present invention.

The present invention also encompasses complexes formed by a BCA polynucleotide and a binding partner, and encompasses complexes formed by a BCA polypeptide and a binding partner. A binder partner can be, but is not limited to, a polypeptide, carbohydrate or lipid. In a specific embodiment, the present invention encompasses the complex of BCA1/Smurf2. In a specific embodiment, the present invention encompasses the complex of BCA1/AIP4. In a specific embodiment, the present invention encompasses the complex of BCA1/Smad2. In other specific embodiments, the present invention encompasses the complexes of BCA1 and polypeptides, and fragments thereof, encoded by a cDNA listed in Table 1. The present invention also provides for methods of identifying and isolating such binding partners, using techniques well known in the art.

In addition, the BCA polynucleotides and polypeptides of the invention, including antibodies directed against polypeptides of the invention, can be used for purposes independent of the role of the gene products as described above. For example, gene products of the invention, including BCA peptide fragments, as well as specific antibodies thereto, can be used for construction of fusion proteins to facilitate recovery, detection, or localization of another protein of interest. In addition, genes and gene products of the invention can be used for genetic mapping. Finally, BCA nucleic acids and gene products have generic uses, such as supplemental sources of nucleic acids, proteins and amino acids for food additives or cosmetic products.

Additionally, the present invention contemplates use of the BCA polynucleotides, BCA polypeptides, BCA agonists and/or BCA antagonists of the invention to screen, diagnose, stage, prevent and/or treat disorders characterized by aberrant expression or activity of a BCA nucleic acid and/or polypeptide of the invention. Such disorders include, but are not limited to, hormone-sensitive cancers (e.g., cancer of the breast, ovary, uterus, prostate, testis, skin and brain).

### 5.1. Antisense Oligonucleotides.

The present invention encompasses BCA antisense polynucleotides, i.e., molecules which are complementary to a sense nucleic acid encoding a polypeptide of the invention, e.g., complementary to the coding strand of a double-stranded cDNA molecule or complementary to an mRNA sequence. The antisense nucleic acid can be complementary to an entire coding strand, or to only a portion thereof, e.g., all or part of the protein coding region (or open reading frame). An antisense polynucleotide can be antisense to all or part of a non-coding region of the coding strand of a nucleotide sequence encoding a polypeptide of the invention. The non-coding regions are the 5′ and 3′ sequences which flank the coding region and are not typically translated into amino acids.

The antisense oligonucleotides of the invention can be DNA or RNA or chimeric mixtures, derivatives, or variants thereof. The oligonucleotide can be modified at the base moiety, sugar moiety, or phosphate backbone, which can, for example, improve the oligonucleotide's pharmacokinetics and/or affect an oligonucleotide's hybridization to the target mRNA. The oligonucleotide can include other appended groups, such as for example, peptides (e.g., for targeting host cell receptors in vivo), agents facilitating transport across the cell membrane (See, e.g., Letsinger et al., 1989, “Cholesteryl-conjugated oligonucleotides: synthesis, properties, and activity as inhibitors of replication of human immunodeficiency virus in cell culture”, Proc Natl Acad Sci. 86:6553-6556; Lemaitre et al., 1987, “Specific antiviral activity of a poly(L-lysine)-conjugated oligodeoxyribonucleotide sequence complementary to vesicular stomatitis virus N protein mRNA initiation site”, Proc Natl Acad Sci. 84:648-652; PCT Publication No. WO 88/09810), hybridization-triggered cleavage agents (See, e.g., van der Krol, 1988, “Modulation of eukaryotic gene expression by complementary RNA or DNA sequences”, Biotechniques 6:958-976), and intercalating agents (See, e.g., Zon, 1988, “Oligonucleotide analogues as potential chemotherapeutic agent”, Pharm Res. 5:539-549). To this end, the oligonucleotide may be conjugated to another molecule, which includes, but is not limited to, a peptide, hybridization triggered cross-linking agent, transport agent, and hybridization-triggered cleavage agent.

An antisense oligonucleotide can be, for example, about 8, 10, 15, 20, 25, 30, 35, 40, 45 or 50 nucleotides or more in length. In one embodiment, the antisense oligonucleotide comprises sequences complementary to the 5′ untranslated region or the 3′ untranslated region. An antisense nucleic acid of the invention can be constructed using chemical synthesis and enzymatic ligation reactions using procedures known in the art.

Various well-known modifications to the DNA molecules may be introduced as a means of increasing intracellular stability and half-life. For example, an antisense polynucleotide (e.g., an antisense oligonucleotide) can be chemically synthesized using naturally occurring nucleotides or variously modified nucleotides. Possible modifications include but are not limited to, the addition of flanking sequences of ribo- or deoxy-nucleotides to the 5′ and/or 3′ ends of the molecule or the use of phosphorothioate or 2′ O-methyl rather than phosphodiester linkages within the oligodeoxyribonucleotide backbone. Examples of modified nucleotides which can be used to generate a BCA antisense polynucleotide include, but are not limited to, 1-methylguanine, 1-methylinosine, 2,2-dimethylguanine, 2-methyladenine, 2-methylguanine, 2-methylthio-N6-isopentenyladenine, 2-thiocytosine, 2-thiouracil, 2,6-diaminopurine, 3-methylcytosine, 3-(3-amino-3-N-2-carboxypropyl) uracil, 4-acetylcytosine, 4-thiouracil, 5-fluorouracil, 5-bromouracil, 5-chlorouracil, 5-iodouracil, 5-(carboxyhydroxylmethyl) uracil, 5-carboxymethylaminomethyl-2-thiouridine, 5-carboxymethylaminomethyluracil, 5-methylcytosine, 5-methoxyaminomethyl-2-thiouracil, 5-methylaminomethyluracil, 5′-methoxycarboxymethyluracil, 5-methoxyuracil, 5-methyl-2-thiouracil, 5-methyl-2-thiouracil, 5-methyluracil, hypoxanthine, 7-methylguanine, beta-D-galactosylqueosine, beta-D-mannosylqueosine, dihydrouracil, inosine, N6-isopentenyladenine, N6-adenine, uracil-5-oxyacetic acid (v), pseudouracil, queosine, wybutoxosine, xanthine, uracil-5-oxyacetic acid methylester, (acp3)w and uracil-5-oxyacetic acid (v).

In another embodiment, the antisense oligonucleotide comprises a modified sugar moieties, which includes, but is not limited to, 2-fluoroarabinose, arabinose, hexose, and xylulose.

In another embodiment, the antisense oligonucleotide comprises a modified phosphate backbone, which includes, but is not limited to, phosphorothioate, phosphorodithioate, phosphoramidothioate, phosphoramidate, phosphorodiamidate, methylphosphonate, alkyl phosphotriester, formacetal, and analogs thereof.

In yet another embodiment, the antisense oligonucleotide is an α-anomeric oligonucleotide. An α-anomeric oligonucleotide can form double-stranded hybrids with complementary RNA, but in contrast to the usual β-oligonucleotides, the nucleotide strands run parallel to each other (Gautier et al., 1987, Nucleic Acids Res. 15:6625-6641). The oligonucleotide can be a 2′-O-methylribonucleotide (Inoue et al., 1987, Nucleic Acids Res. 15:6131-6148) or a chimeric RNA-DNA analogue (Inoue et al., 1987, FEBS Lett. 215:327-330).

The invention contemplates use of one or more BCA antisense oligonucleotides, or a variant thereof. Antisense oligonucleotides suitable for use in the invention are, for example, 10 to 20, 20 to 50, 50 to 75, 75 to 100, 100 to 200, 200 to 300, or 300-400 bases in length. The target sequences can be RNA or DNA, and can be single-stranded or double-stranded. Target molecules include, but are not limited to, pre-mRNA, mRNA and DNA. In a one embodiment, the target molecule is a BCA mRNA. In a preferred embodiment, the target molecule is BCA pre-mRNA or BCA mRNA. An antisense polynucleotide of the invention preferentially hybridizes to any one site anywhere along a BCA pre-mRNA or mRNA. In another embodiment, a BCA antisense oligonucleotide is selected from the group consisting of oligonucleotides that hybridize to the translation initiation site, donor splicing site, acceptor splicing site, sites for transportation, or sites for degradation of a BCA pre-mRNA or mRNA.

The antisense polynucleotides of the invention are typically administered to a subject or generated in situ such that they hybridize with or bind to cellular mRNA and/or genomic DNA encoding a selected polypeptide of the invention to thereby inhibit expression, e.g., by inhibiting transcription and/or translation. The hybridization can be by conventional nucleotide complementarity to form a stable duplex, or, for example, in the case of an antisense polynucleotide which binds to DNA duplexes, through specific interactions in the major groove of the double helix. An example of a route of administration of antisense polynucleotides of the invention includes direct injection at a tissue site. Alternatively, antisense polynucleotides can be modified to target selected cells and then administered systemically. For example, for systemic administration, antisense molecules can be modified such that they specifically bind to receptors or antigens expressed on a selected cell surface, e.g., by linking the antisense polynucleotides to peptides or antibodies which bind to cell surface receptors or antigens.

In one embodiment, a BCA antisense oligonucleotide is substantially complementary to a portion of a BCA pre-mRNA or mRNA, or to a portion of a pre-mRNA or mRNA that is related to a BCA gene of the invention. In another embodiment, a BCA antisense oligonucleotide hybridizes to a portion of the translation-initiation site of the pre-mRNA coding strand. In yet another embodiment, a BCA antisense oligonucleotide hybridizes to a portion of the pre-mRNA coding strand that comprises the translation-initiation site of the human BCA gene. In yet another embodiment, a BCA antisense oligonucleotide comprises a TAC sequence which is complementary to the AUG initiation sequence of a BCA pre-mRNA or RNA.

In another embodiment, a BCA antisense oligonucleotide hybridizes to a portion of the splice donor site of the pre-mRNA coding strand for the human BCA gene. In another embodiment, this nucleotide comprises a CA sequence, which is complementary to the GT splice donor sequence of a BCA gene, and hybridizes to portions of a BCA gene coding strand flanking the splice donor site.

In yet another embodiment, a BCA antisense oligonucleotide hybridizes to a portion of the splice acceptor site of the pre-mRNA coding strand for the human BCA gene. In another embodiment, this nucleotide comprises a TC sequence, which is complementary to the AG splice acceptor sequence of a BCA gene, and hybridizes to portions of a BCA gene coding strand flanking the splice acceptor site. In another embodiment, a BCA antisense oligonucleotide hybridizes to portions of the pre-mRNA or mRNA involved in splicing, transport or degradation.

One of average skill in the art can recognize that antisense oligonucleotides suitable for use in the invention may also be substantially complementary to other sites along a BCA pre-mRNA or mRNA. The skilled artisan will also appreciate that antisense oligonucleotides, which hybridize to a portion of a BCA pre-mRNA or mRNA whose sequence does not commonly occur in transcripts from unrelated genes, are preferable so as to maintain treatment specificity.

The design of the sequence of a BCA antisense oligonucleotide can also be determined by empirical testing and assessment of clinical effectiveness, regardless of its degree of sequence homology to, or hybridization with, a BCA gene, BCA pre-mRNA, BCA mRNA, or BCA-related nucleotide sequences. One of ordinary skill in the art will appreciate that BCA antisense oligonucleotides having, for example, less sequence homology, greater or fewer modified nucleotides, or longer or shorter lengths, compared to those of the above embodiments, but which nevertheless demonstrate responses in clinical treatments, are also within the scope of the invention.

The antisense oligonucleotides may be RNA or DNA, or variants thereof. The particular form of antisense oligonucleotide may affect the oligonucleotide's pharmacokinetic parameters such as bioavailability, metabolism, and half-life. As such, where appropriate, the invention contemplates antisense oligonucleotide derivatives having properties that improve cellular uptake, enhance nuclease resistance, improve binding to the target sequence, or increase cleavage or degradation of the target sequence. The antisense oligonucleotides may comprise bases comprising, for example, phosphorothioates or methylphosphonates. The antisense oligonucleotides, instead, can be mixed oligonucleotides comprising combinations of phosphodiester, phosphorothioate, and/or methylphosphonate nucleotides, among others. Such oligonucleotides may possess modifications which comprise, but are not limited to, 2-O′-alkyl or 2-O′-halo sugar modifications, backbone modifications (e.g., methylphosphonate, phosphorodithioate, phosphordithioate, formacetal, 3′-thioformacetal, sulfone, sulfamate, nitroxide backbone, morpholino derivatives and peptide nucleic acid (“PNA”) derivatives), or variants wherein the base moieties have been modified. In another embodiment, antisense oligonucleotides comprise conjugates of the oligonucleotides and variants thereof (Goodchild, 1990, “Conjugates of oligonucleotides and modified oligonucleotides: a review of their synthesis and properties”, Bioconjug Chem. 1:165-187).

In one embodiment, the deoxyribose phosphate backbone of a polynucleotide of the invention can be modified to incorporate peptide nucleic acids (“PNAs”) (See, e.g., Hyrup et a., 1996, Bioorganic & Medicinal Chemistry 4: 5-23). As used herein, PNAs refer to nucleic acid mimics, in which the deoxyribose phosphate backbone is replaced by a pseudopeptide backbone. The neutral backbone of PNAs allows for specific hybridization to DNA and RNA under conditions of low ionic strength. The synthesis of PNA oligomers can be performed using standard solid phase peptide synthesis protocols as described in Hyrup et al., 1996 supra; Perry-O'Keefe et al., 1996, Proc Natl Acad Sci. 93:14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used for analyzing gene mutations by, for example, PNA-directed PCR clamping, or as artificial restriction enzymes when used in combination with other enzymes, such as for example, S1 nucleases (Hyrup et al., 1996 supra), or as probes or primers for DNA sequence and hybridization (Hyrup et al., 1996, supra; Perry-O'Keefe et al., 1996, supra).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup et al., 1996, supra). The synthesis of PNA-DNA chimeras can be performed as described in Hyrup et al., 1996, supra, and Finn et al., 1996, Nucleic Acids Res. 24:3357-3363. For example, a DNA chain can be synthesized on a solid support using standard phosphoramidate coupling chemistry and modified nucleoside analogs. Compounds such as 5′-(4-methoxytrityl)amino-5′-deoxy-thymidine phosphoramidate can be used as a link between the PNA and the 5′ end of DNA (Mag et al., 1989, Nucleic Acids Res. 17:5973-5988). PNA monomers are then coupled in a stepwise manner to produce a chimeric molecule with a 5′ PNA segment and a 3′ DNA segment (Finn et al., 1996, Nucleic Acids Res. 24:3357-3363). Alternatively, chimeric molecules can be synthesized with a 5′ DNA segment and a 3′ PNA segment (Peterser et al., 1975, Bioorganic Med. Chem. Lett. 5:1119-1124).

For in vivo therapeutic use, a phosphorothioate derivative of a BCA antisense oligonucleotide can be useful, at least partly because of greater resistance to degradation. In one embodiment, a BCA antisense oligonucleotide is a hybrid oligonucleotide comprising phosphorothioate bases. In another embodiment, a BCA antisense oligonucleotide comprises at least one phosphorothioate linkage. In yet another embodiment, a BCA antisense oligonucleotide is comprised entirely of phosphorothioate linkages. Methods for preparing oligonucleotide variants are known in the art. See, e.g., Stein et al., 1988, Nucleic Acids Res. 16:3209-3221 (phosphorothioate); Blake et al., 1985, Biochemistry 24:6132-38 (methylphosphonate); Morvan et al., 1986, Nucleic Acids Res. 14:5019-5032 (alphadeoxynucleotides); Monia et al., 1993, “Evaluation of 2′-modified oligonucleotides containing 2′ deoxy gaps as antisense inhibitors of gene expression”, J. Biol. Chem. 268:14514-22 (2′-O-methyl-ribonucleosides); Asseline et al., 1984, Proc. Natl Acad. Sci. USA 81:3297-3301 (acridine); Knorre et al., 1985, Biochimie 67:783-9; Vlassov et al., 1986, Nucleic Acids Res. 14:4065-76 (N-2-chlorocethylamine and phenazine); Webb et al., 1986, Nucleic Acids Res. 14:7661-74 (5-methyl-N⁴-N⁴-ethanocytosine); Boutorin et al., 1984, FEBS Letters 172:43-6 (Fe-ethylenediamine tetraacetic acid (“EDTA”) and analogues); Chi-Hong et al., 1986, Proc Natl Acad Sci. 83:7147-51 (5-glycylamido-1, 10-o-phenanthroline); and Chu et al., 1985, Proc Natl Acad Sci. 82:963-967 (diethylenetriamine-pentaacetic acid (“DTPA”) derivatives).

The effective dose of BCA antisense oligonucleotide to be administered during a treatment cycle ranges from about 0.01 to 0.1, 0.1 to 1, or 1 to 10 mg/kg/day. The dose of BCA antisense oligonucleotide to be administered can be dependent on the mode of administration. For example, intravenous administration of a BCA antisense oligonucleotide would likely result in a significantly higher full body dose than a full body dose resulting from a local implant containing a pharmaceutical composition comprising BCA antisense oligonucleotide. In one embodiment, a BCA antisense oligonucleotide is administered subcutaneously at a dose of 0.01 to 10 mg/kg/day. In another embodiment, a BCA antisense oligonucleotide is administered intravenously at a dose of 0.01 to 10 mg/kg/day. In yet another embodiment, a BCA antisense oligonucleotide is administered locally at a dose of 0.01 to 10 mg/kg/day. It will be evident to one skilled in the art that local administrations can result in lower total body doses. For example, local administration methods such as intratumor administration, intraocular injection, or implantation, can produce locally high concentrations of BCA antisense oligonucleotide, but represent a relatively with respect to total body weight. Thus, in such cases, local administration of a BCA antisense oligonucleotide is contemplated to result in a total body dose of about 0.01 to 5 mg/kg/day.

In another embodiment, a particularly high dose of BCA antisense oligonucleotide, which ranges from about 10 to 50 mg/kg/day, is administered during a treatment cycle.

Moreover, the effective dose of a particular BCA antisense oligonucleotide may depend on additional factors, including the type of disease, the disease state or stage of disease, the oligonucleotide's toxicity, the oligonucleotide's rate of uptake by cancer cells, as well as the weight, age, and health of the individual to whom the antisense oligonucleotide is to be administered. Because of the many factors present in vivo that may interfere with the action or biological activity of a BCA antisense oligonucleotide, one of ordinary skill in the art can appreciate that an effective amount of a BCA antisense oligonucleotide may vary for each individual.

In another embodiment, a BCA antisense oligonucleotide is at a dose which results in circulating plasma concentrations of a BCA antisense oligonucleotide which is at least 50 nM (nanomolar). As will be apparent to the skilled artisan, lower or higher plasma concentrations of a BCA antisense oligonucleotide may be preferred depending on the mode of administration. For example, plasma concentrations of a BCA antisense oligonucleotide of at least 50 nM can be appropriate in connection with intravenous, subcutaneous, intramuscular, controlled release, and oral administration methods, to name a few. In another example, relatively low circulating plasma levels of a BCA antisense oligonucleotide can be desirable, however, when using local administration methods such as, for example, intratumor administration, intraocular administration, or implantation, which nevertheless can produce locally high, clinically effective concentrations of BCA antisense oligonucleotide.

The high dose may be achieved by several administrations per cycle. Alternatively, the high dose may be administered in a single bolus administration. A single administration of a high dose may result in circulating plasma levels of BCA antisense oligonucleotide that are transiently much higher than 50 nM.

Additionally, the dose of a BCA antisense oligonucleotide may vary according to the particular BCA antisense oligonucleotide used. The dose employed is likely to reflect a balancing of considerations, among which are stability, localization, cellular uptake, and toxicity of the particular BCA antisense oligonucleotide. For example, a particular chemically modified BCA antisense oligonucleotide may exhibit greater resistance to degradation, or may exhibit higher affinity for the target nucleic acid, or may exhibit increased uptake by the cell or cell nucleus; all of which may permit the use of low doses. In yet another example, a particular chemically modified BCA antisense oligonucleotide may exhibit lower toxicity than other antisense oligonucleotides, and therefore can be used at high doses. Thus, for a given BCA antisense oligonucleotide, an appropriate dose to administer can be relatively high or relatively low. Appropriate doses would be appreciated by the skilled artisan, and the invention contemplates the continued assessment of optimal treatment schedules for particular species of BCA antisense oligonucleotides. The daily dose can be administered in one or more treatments.

A “low dose” or “reduced dose” refers to a dose that is below the normally administered range, i.e., below the standard dose as suggested by the Physicians' Desk Reference, 54^{th} Edition (2000) or a similar reference. Such a dose can be sufficient to inhibit cell proliferation, or demonstrates ameliorative effects in a human, or demonstrates efficacy with fewer side effects as compared to standard cancer treatments. Normal dose ranges used for particular therapeutic agents and standard cancer treatments employed for specific diseases can be found in the Physicians' Desk Reference 54^{th} Edition (2000) or in Cancer: Principles & Practice of Oncology, DeVita, Jr., Hellman, and Rosenberg (eds.) 2nd edition, Philadelphia, Pa.: J.B. Lippincott Co., 1985.

Reduced doses of BCA polynucleotide, BCA polypeptide, BCA antagonist, and/or combination therapeutic can demonstrate reduced toxicity, such that fewer side effects and toxicities are observed in connection with administering a BCA antagonist and one or more cancer therapeutics for shorter duration and/or at lower dosages when compared to other treatment protocols and dosage formulations, including the standard treatment protocols and dosage formulations as described in the Physicians' Desk Reference. 54^{th} Edition (2000) or in Cancer: Principles & Practice of Oncology, DeVita, Jr., Hellman, and Rosenberg (eds.) 2nd edition, Philadelphia, Pa.: J.B. Lippincott Co., 1985.

A “treatment cycle” or “cycle” refers to a period during which a single therapeutic or sequence of therapeutics is administered. In some instances, one treatment cycle may be desired, such as for example, in the case where a significant therapeutic effect is obtained after one treatment cycle. The present invention contemplates at least one treatment cycle, generally preferably more than one treatment cycle.

Other factors to be considered in determining an effective dose of a BCA antisense oligonucleotide include whether the oligonucleotide will be administered in combination with other therapeutics. In such cases, the relative toxicity of the other therapeutics may indicate the use of a BCA antisense oligonucleotide at low doses. Alternatively, treatment with a high dose of BCA antisense oligonucleotide can result in combination therapies with reduced doses of therapeutics. In a specific embodiment, treatment with a particularly high dose of BCA antisense oligonucleotide can result in combination therapies with greatly reduced doses of cancer therapeutics. For example, treatment of a patient with 10, 20, 30, 40, or 50 mg/kg/day of a BCA antisense oligonucleotide can further increase the sensitivity of a subject to cancer therapeutics. In such cases, the particularly high dose of BCA antisense oligonucleotide is combined with, for example, a greatly shortened radiation therapy schedule. In another example, the particularly high dose of a BCA antisense oligonucleotide produces significant enhancement of the potency of cancer therapeutic agents.

Additionally, the particularly high doses of BCA antisense oligonucleotide may further shorten the period of administration of a therapeutically effective amount of BCA antisense oligonucleotide and/or additional therapeutic, such that the length of a treatment cycle is much shorter than that of the standard treatment.

The invention contemplates other treatment regimens depending on the particular BCA antisense oligonucleotide to be used, or depending on the particular mode of administration, or depending on whether a BCA antisense oligonucleotide is administered as part of a combination therapy, e.g., in combination with a cancer therapeutic agent. The daily dose can be administered in one or more treatments.

Alternatively, the antisense nucleic acid can be produced biologically using an expression vector into which a nucleic acid has been subcloned in an antisense orientation, such that the RNA transcribed from the inserted nucleic acid will be of an antisense orientation to a target polynucleotide of interest. The antisense polynucleotides can also be delivered to cells using the vectors described herein. In a particular embodiment, sufficient intracellular concentrations of the antisense molecules of the invention are obtained by using vector constructs in which the antisense polynucleotide is placed under the control of a strong pol II or pol III promoter (See Section 5.6.17 infra).

### 5.2. Isolated Polypeptides

One aspect of the invention pertains to isolated BCA polypeptides, variants thereof (e.g., biologically active portions such as SH2 domains), as well as variants suitable for use as immunogens to raise antibodies directed against a BCA polypeptide of the invention. In one embodiment, the native polypeptide can be isolated, using standard protein purification techniques, from cells or tissues expressing a BCA polypeptide. In a preferred embodiment, polypeptides of the invention are produced from expression vectors by recombinant DNA techniques. In another preferred embodiment, a polypeptide of the invention is synthesized chemically using standard peptide synthesis techniques.

An isolated or purified protein or biologically active portion thereof is substantially free of cellular material or other contaminating proteins from the cell or tissue source from which the protein is derived, or substantially free of chemical precursors or other chemicals when chemically synthesized. The language “substantially free” indicates protein preparations in which the protein is separated from cellular components of the cells from which it is isolated or recombinantly produced. Thus, protein that is substantially free of cellular material includes protein preparations having less than 20%, 10%, or 5% (by dry weight) of a contaminating protein. Similarly, when an isolated BCA polypeptide of the invention is recombinantly produced, it is substantially free of culture medium. When the BCA polypeptide is produced by chemical synthesis, it is preferably substantially free of chemical precursors or other chemicals.

Biologically active portions of a polypeptide of the invention include polypeptides comprising amino acid sequences identical to or derived from the amino acid sequence of the protein, such that the variants sequences comprise conservative substitutions or truncations (e.g., amino acid sequences comprising fewer amino acids than those shown in any of SEQ ID NO:2, 4, 6, 8, 10, 12, and 14, but which maintain a high degree of homology to the remaining amino acid sequence). Typically, biologically active portions comprise a domain or motif with at least one activity of the corresponding protein. Domains or motifs include, but are not limited to, a biologically active portion of a protein of the invention can be a polypeptide which is, for example, at least 10, 25, 50, 100, 200, 300, 400 or 500 amino acids in length. Polypeptides of the invention can comprise, for example, a BCA extracellular domain, transmembrane domain, BCA intracellular domain, BCA signal peptide, phosphorylation sites (e.g., protein kinase C site, casein kinase 2 site), SH2 domains, Ring H2 domains, NPXXY motifs, glycosylation signals, subcellular localization signals, myristylation sites, or protein degradation signals.

In one embodiment, a BCA polypeptide may have a signal sequence (or signal peptide or secretion signal) which refers to a peptide of at least about 15 or 20 amino acid residues in length which occurs at the N-terminus of secretory and membrane-bound proteins and which comprises at least about 70% hydrophobic amino acid residues such as alanine, leucine, isoleucine, phenylalanine, proline, tyrosine, tryptophan, or valine. A signal sequence serves to direct a protein comprising such a sequence to a lipid bilayer. A signal sequence is usually cleaved during processing of the mature protein. In a preferred embodiment, a signal sequence comprises at least about 10 to 40 amino acid residues, preferably about 19-34 amino acid residues, and has at least about 60-80%, more preferably at least 65-75%, and more preferably at least 70% hydrophobic residues.

In another embodiment, a BCA polypeptide may have a transmembrane domain, which is an amino acid sequence comprising at least about 20 to 40 amino acid residues in length and having hydrophobic amino acid residues, such as alanine, leucine, isoleucine, phenylalanine, proline, tyrosine, tryptophan, or valine. In a preferred embodiment, a transmembrane domain comprises at least about 20 to 40 amino acid residues, preferably 25-30 amino acid residues, and has at least about 60-80% hydrophobic residues.

In particular embodiments, the invention encompasses a fragment of a human BCA1 polypeptide comprising at least 5 consecutive amino acids of a human BCA1 polypeptide, wherein said fragment is a portion of a domain selected from the group consisting of a RING H2 finger, PY motif, glycosylation site, phosphorylation site, SH2-binding motif, open-reading frame, exon 1, exon 2, exon 3, intron 1, intron 2, 5′ untranslated region, and 3′ untranslated region.

In particular embodiments, the invention encompasses a fragment of a human BCA2 polypeptide comprising at least 5 consecutive amino acids of a human BCA2 polypeptide, wherein said fragment is a portion of a domain selected from the group consisting of a RING H2, NPXXY motif, PXXP motif, zinc finger, glycosylation site, phosphorylation site, SH3-binding motif, open-reading frame, exon 1, exon 2, exon 3, exon 4, exon 5, exon 6, exon 7, exon 8, exon 9, intron 1, intron 2, intron 3, intron 4, intron 5, intron 6, intron 7, intron 8, 5′ untranslated region, and 3′ untranslated region.

In particular embodiments, the invention encompasses a fragment of a human BCA3 polypeptide comprising at least 5 consecutive amino acids of a human BCA3 polypeptide, wherein said fragment is a portion of a domain selected from the group consisting of a SH2 site YYSS, SH2 site YSSV, SH2 site YHRG, SH2 site YIEV, SH2 site YPGT, SH2 site YSVT, tyrosine phosphorylation site, RTMAEFMDY, glycosylation site, phosphorylation site, tyrosine phosphorylation motif, SH2-binding motif, open-reading frame, open-reading frame lacking exon 3, open-reading frame lacking exon 3 and exon 5, exon 1, exon 2, exon 3, exon 4, exon 5, exon 6, exon 7, intron 1, intron 2, intron 3, intron 4, intron 5, intron 6, 5′ untranslated region, and 3′ untranslated region.

Preferred polypeptides consist of an amino acid sequence of SEQ ID NO:2, 4, 6, 8, 10, 12, and 14. Other useful polypeptides are substantially identical (e.g., at least 65%, preferably 75%, 85%, 90%, 95%, or 99%) to any of SEQ ID NO:2, 4, 6, 8, 10, 12, and 14. In certain embodiments, the invention provides fragments of the amino acid sequence wherein the percent identity is determined over amino acid sequences of identical size to the fragment. In other embodiments, the invention provides a polypeptide comprising an amino acid sequence that has at least 90% identity to the fragments of domains identified in the BCA polypeptides, wherein the percent identity is determined over an amino acid sequence of identical size to said fragment.

The determination of percent identity between two sequences can be accomplished using a mathematical algorithm. A preferred, non-limiting example of a mathematical algorithm utilized for the comparison of two sequences is the algorithm of Karlin and Altschul (1990, Proc Natl Acad Sci. 87:2264-2268), modified as in Karlin and Altschul (1993, Proc Natl Acad Sci. 90:5873-5877). Such an algorithm is incorporated into the NBLAST and XBLAST programs of Altschul et al. (1990, J. Mol. Biol. 215:403-410). BLAST nucleotide searches can be performed with the NBLAST program, score=100, wordlength=12 to obtain nucleotide sequences homologous to a polynucleotides of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997, Nucleic Acids Res. 25:3389-3402). Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. See http://www.ncbi.nlm.nih.gov.

Another preferred, non-limiting example of a mathematical algorithm utilized for the comparison of sequences is the algorithm of Myers and Miller, (1988, CABIOS 4:11-17). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti (1994, Comput. Appl. Biosci. 10:3-5); and FASTA described in Pearson and Lipman (1988, 85:2444-2448). Within FASTA, ktup is a control option that sets the sensitivity and speed of the search. If ktup=2, similar regions in the two sequences being compared are found by looking at pairs of aligned residues; if ktup=1, single aligned amino acids are examined. ktup can be set to 2 or 1 for protein sequences, or from 1 to 6 for DNA sequences. The default if ktup is not specified is 2 for proteins and 6 for DNA. For a further description of FASTA parameters, see http://bioweb.pasteur.fr/docs/man/man/fasta.1.htm1#sect2.

The percent identity between two sequences can be determined using techniques similar to those described above, with or without allowing gaps. In calculating percent identity, only exact matches are counted. However, conservative substitutions should be considered in evaluating sequences that have a low percent identity with the BCA sequences disclosed herein.

The invention also provides chimeric or fusion proteins. As used herein, a “chimeric protein” or “fusion protein” comprises all or part (preferably biologically active) of a polypeptide of the invention fused in-frame to a second polypeptide. In one embodiment, the second polypeptide is a heterologous polypeptide. In another embodiment, the second polypeptide is different from, but derived from the same, polypeptide to which it is attached. The second polypeptide can be fused to the N-terminus or C-terminus of the polypeptide of the invention.

In another embodiment, the protein of the invention can be expressed as a dimer of itself. In this embodiment, a first domain of the protein is fused in frame to the same domain by a linker region. The linker can be a short flexible segment of amino acids, for example GGPGG (SEQ ID NO.: 23) or GPPGG (SEQ ID NO.: 24), or a longer segment as needed. Alternatively, the first domain of the protein can be fused to a second domain of the protein, which is different than the first domain.

One useful fusion protein is a GST fusion protein in which the polypeptide of the invention is fused to the C-terminus of GST sequences. Such fusion proteins can facilitate the purification of a recombinant polypeptide of the invention.

In another embodiment, the fusion protein comprises a heterologous signal sequence at its N-terminus. For example, the native signal sequence of a polypeptide of the invention can be removed and replaced with a signal sequence from another protein. For example, the gp67 secretory sequence of the baculovirus envelope protein can be used as a heterologous signal sequence (Ausubel et al., Current Protocols in Molecular Biology, John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

In yet another embodiment, the fusion protein is an immunoglobulin fusion protein in which all or part of a polypeptide of the invention is fused with sequences derived from a member of the immunoglobulin protein family. The immunoglobulin fusion proteins of the invention can be incorporated into pharmaceutical compositions and administered to a subject to inhibit an interaction between a ligand (soluble or membrane-bound) and a protein on the surface of a cell (receptor), to thereby suppress signal transduction in vivo. The immunoglobulin fusion protein can be used to affect the bioavailability of a cognate ligand of a polypeptide of the invention. Inhibition of ligand/receptor interaction can be useful therapeutically, both for treating proliferative and differentiative disorders and for modulating (e.g., promoting or inhibiting) cell survival. Moreover, the immunoglobulin fusion proteins of the invention can be used as immunogens to produce antibodies directed against a polypeptide of the invention in a subject, to purify ligands and in screening assays to identify molecules which inhibit the interaction of receptors with ligands. The immunoglobulin fusion protein can, for example, comprise a portion of a polypeptide of the invention fused with the amino-terminus or the carboxyl-terminus of an immunoglobulin constant region, as disclosed in U.S. Pat. No. 5,714,147, U.S. Pat. No. 5,116,964, U.S. Pat. No. 5,514,582, and U.S. Pat. No. 5,455,165.

Chimeric and fusion proteins of the invention can be produced by standard recombinant DNA techniques. In one embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the invention can be cloned into such an expression vector such that the fusion moiety is linked in-frame to the polypeptide of the invention.

A signal sequence of a polypeptide of the invention can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides comprise processing sites that allow cleavage of the signal sequence from the mature proteins as they pass through the secretory pathway. Thus, the invention pertains to the described polypeptides having a signal sequence, as well as to the signal sequence itself and to the polypeptide in the absence of the signal sequence (i.e., the cleavage products). In one embodiment, a polynucleotide encoding a signal sequence of the invention can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as a GST domain.

In another embodiment, the signal sequences of the present invention can be used to identify regulatory sequences, e.g., promoters, enhancers, repressors. Since signal sequences are the most amino-terminal sequences of a peptide, it is expected that the nucleic acids which flank the signal sequence on its amino-terminal side will be regulatory sequences which affect transcription. Thus, a nucleotide sequence which encodes all or a portion of a signal sequence can be used as a probe to identify and isolate signal sequences and their flanking regions, and these flanking regions can be studied to identify regulatory elements therein.

The present invention also pertains to variants of the polypeptides of the invention. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a protein of the invention which function as either agonists (mimetics) or as antagonists can be identified by screening combinatorial libraries of mutants, e.g., truncation mutants, of the protein of the invention for agonist or antagonist activity. In one embodiment, a library of variants is generated by combinatorial mutagenesis at the nucleic acid level. Such a library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the polypeptides of the invention from a degenerate oligonucleotide sequence. Methods for synthesizing degenerate oligonucleotides are known in the art (See, e.g., Narang, 1983, Tetrahedron 39:3; Itakura et al., 1984, Annu Rev Biochem. 53:323; Itakura et al., 1984, Science 198:1056; Ike et al., 1983, Nucleic Acid Res. 11:477).

In addition, libraries of fragments of the coding sequence of a polypeptide of the invention can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected.

Moreover, variants of a polypeptide of the invention can be produced by directed evolution techniques. For example, recursive ensemble mutagenesis, a technique which enhances the frequency of functional mutants in the libraries, can be used in combination with the screening assays to identify variants of a protein of the invention (See, e.g., Arkin and Yourvan, 1992, Proc Natl Acad Sci. 89:7811-7815; Delgrave et al., 1993, Protein Engineering 6:327-331).

The polypeptides of the invention can exhibit post-translational modifications, including, but not limited to glycosylations, (e.g., N-linked or O-linked glycosylations), myristylations, palmitylations, acetylations and phosphorylations (e.g., serine/threonine or tyrosine). In one embodiment, the polypeptides of the invention exhibit reduced levels of O-linked glycosylation and/or N-linked glycosylation relative to endogenously expressed. In another embodiment, the polypeptides of the invention dt) not exhibit O-linked glycosylation or N-linked glycosylation.

The polypeptides of the invention can, for example, include modifications that can increase such attributes as stability, half-life, ability to enter cells and aid in administration, e.g., in vivo administration of the polypeptides of the invention. For example, polypeptides of the invention can comprise a protein transduction domain of the HIV TAT protein (See, e.g., Schwarze et al., 1999, Science 285:1569-1572), thereby facilitating delivery of polypeptides of the invention into cells.

The polypeptides of the invention also encompasses complexes comprising BCA1 polypeptide and at least one binding partner selected from the group consisting of a gene product of AIP4, Smurf2, polyubiquitin UbC, DUT, EPS15, ZBRK1, chromosome 19 open reading frame 5, AMSH, PLAT, TOM1L2, FLJ11626, clone 155, VIM, NVS, clone 287, clone 292, and POLR2J. The polypeptides of the invention also encompasses complexes comprising an amino acid sequence that has 90% sequence identity relative to SEQ ID NO:2, and wherein said polypeptide binds to at least one binding partner selected from the group consisting of a gene product of AIP4, Smurf2, polyubiquitin UbC, DUT, EPS15, ZBRK1, chromosome 19 open reading frame 5, AMSH, PLAT, TOM1L2, FLJ11626, clone 155, VIM, INVS, clone 287, clone 292, and POLR2J.

### 5.3. Recombinant Expression Vectors and Host Cells.

Another aspect of the invention pertains to vectors, preferably expression vectors, comprising a BCA polynucleotide, nucleic acid sequence encoding a BCA polypeptide, BCA agonist, BCA antagonist, inhibitor of a BCA agonist, inhibitor of a BCA antagonist, or a variant thereof. In a particular embodiment, an expression vector comprises a BCA nucleic acid encoding a BCA polypeptide of the invention (or a portion thereof).

As used herein, the term “vector” refers to a polynucleotide capable of transporting another nucleic acid to which it has been linked. One type of vector is a “plasmid”, which refers to a circular double stranded DNA loop into which additional DNA segments can be introduced. Another type of vector is a viral vector, wherein additional DNA segments can be introduced into the viral genome.

Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses).

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operably linked to the polynucleotide to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Gene Expression Technology: Methods in Enzymology, (1990) Academic Press, San Diego, Calif., p. 185. Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

The recombinant expression vectors of the invention can be designed for expression of a polypeptide of the invention in prokaryotic (e.g., E. coli) or eukaryotic cells (e.g., insect cells (using baculovirus expression vectors), yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors comprising constitutive or inducible promoters directing the expression of either fusion or non-fusion proteins. Fusion vectors add a number of amino acids to a protein encoded therein, usually to the amino terminus of the recombinant protein. Such fusion vectors typically serve at least three purposes: 1) to increase expression of recombinant protein; 2) to increase the solubility of the recombinant protein; and/or 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988, Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., 1988, Gene 69:301-315) and pET 11d (Studier et al., Gene Expression Technology: Methods in Enzymology 185, Academic Press, San Diego, Calif. (1990) p. 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21 (DE3) or HMS174(DE3) from a resident λ prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, Gene Expression Technology: Methods in Enzymology 185, Academic Press, San Diego, Calif. (1990) p. 119-128). Another strategy is to alter the sequence of the nucleic acid to be inserted into an expression vector so that the individual codons for each amino acid are those preferentially utilized in E. coli (Wada et al., 1992, Nucleic Acids Res. 20:2111-2118). Such alteration of polynucleotides of the invention can be carried out by standard DNA synthesis techniques.

In another embodiment, the expression vector is a yeast expression vector. Examples of vectors for expression in yeast S. cerevisiae include pYepSec1 (Baldari et al., 1987, EMBO J. 6:229-234), pMFa (Kurjan and Herskowitz, 1982, Cell 30:933-943), pJRY88 (Schultz et al., 1987, Gene 54:113-123), pYES2 (Invitrogen Corp., San Diego, Calif.), and pPicZ (Invitrogen Corp., San Diego, Calif.).

Alternatively, the expression vector is a baculovirus expression vector. Baculovirus vectors available for expression of proteins in cultured insect cells (e.g., Sf9 cells) include the pAc series (Smith et al., 1983, Mol. Cell Biol. 3:2156-2165) and the pVL series (Lucklow and Summers, 1989, Virology 170:31-39).

In yet another embodiment, a BCA nucleic acid of the invention is expressed in mammalian cells using a mammalian expression vector. Examples of mammalian expression vectors include pCDM8 (Seed, 1987, “An LFA-3 cDNA encodes a phospholipid-linked membrane protein homologous to its receptor CD2”, Nature. 840-842) and pMT2PC (Kaufman et al., 1987, “Translational efficiency of polycistronic mRNAs and their utilization to express heterologous genes in mammalian cells”, EMBO J. 6:187-193). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the BCA nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al., 1987, Genes Dev. 1:268-277), lymphoid-specific promoters (Calame and Eaton, 1988, Adv. Immunol. 43:235-275), in particular promoters of T cell receptors (Winoto and Baltimore, 1989, EMBO J. 8:729-733) and immunoglobulins (Banerji et al., 1983, Cell 33:729-740; Queen and Baltimore, 1983, Cell 33:741-748), neuron-specific promoters (e.g., the neurofilament promoter; Byrne and Ruddle, 1989, Proc Natl Acad Sci. 86:5473-5477), pancreas-specific promoters (Edlund et al., 1985, Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss, 1990, Science 249:374-379) and the α-fetoprotein promoter (Campes and Tilghman, 1989, Genes Dev. 3:537-546).

The invention further provides a recombinant expression vector comprising a polynucleotide of the invention cloned into the expression vector in an antisense orientation. That is, the DNA molecule is operably linked to a regulatory sequence in a manner which allows for expression (by transcription of the DNA molecule) of an RNA molecule which is antisense to the mRNA encoding a polypeptide of the invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced.

In another embodiment, the expression characteristics of an endogenous BCA gene within a cell, cell line or microorganism may be modified by inserting a DNA regulatory element heterologous to the endogenous gene of interest into the genome of a cell, stable cell line or cloned microorganism such that the inserted regulatory element is operatively linked with an endogenous BCA gene and controls, modulates or activates the endogenous gene. For example, endogenous genes of the invention which are normally “transcriptionally silent”, i.e., genes which are normally not expressed, or are expressed only at very low levels in a cell line or microorganism, may be activated by inserting a regulatory element which is capable of promoting the expression of a normally expressed gene product in that cell line or microorganism. Alternatively, transcriptionally silent, endogenous genes of the invention may be activated by insertion of a promiscuous regulatory element that works across cell types.

A heterologous regulatory element may be inserted into a stable cell line or cloned microorganism, such that it is operatively linked with and activates expression of an endogenous BCA gene, using techniques, such as targeted homologous recombination, which are well known to those of skill in the art (See, e.g., U.S. Pat. Nos. 5,272,071 and 5,968,502; International Publication Nos. WO 91/06667 and WO 94/12650). Alternatively, non-targeted techniques (e.g., non-homologous recombination) well known in the art can be used (see, e.g., International Publication No. WO 99/15650).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

Accordingly, the present invention provides a host cell having an expression vector comprising a BCA polynucleotide or a nucleic acid sequence encoding a BCA polypeptide, BCA agonist, BCA antagonist, inhibitor of a BCA agonist, inhibitor of a BCA antagonist, or a variant thereof. A host cell can be any prokaryotic (e.g., E. coli) or eukaryotic cell (e.g., insect cells, yeast or mammalian cells). The invention also provides a method for making a BCA polypeptide, e.g., BCA-3 comprising the steps of(a) culturing a cell comprising a recombinant BCA polynucleotide under conditions that allow said BCA. polypeptide to be expressed by said cell; and isolating the expressed BCA polypeptide.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce a BCA polypeptide of the invention. Accordingly, the invention further provides methods for producing a BCA polypeptide of the invention using the host cells of the invention. In one embodiment, the method comprises culturing the host cell of invention (into which a recombinant expression vector encoding a BCA polypeptide of the invention has been introduced) in a suitable medium such that the BCA polypeptide is produced. In another embodiment, the method further comprises isolating the BCA polypeptide from the medium or the host cell.

The host cells of the invention can also be used to produce nonhuman transgenic animals. For example, in one embodiment, a host cell of the invention is a fertilized oocyte or an embryonic stem cell into which a sequence encoding a BCA polypeptide of the invention has been introduced. Such host cells can then be used to create non-human transgenic animals in which exogenous sequences encoding a polypeptide of the invention have been introduced into their genome or homologous recombinant animals in which endogenous sequences encoding a BCA polypeptide of the invention sequences have been altered. Such animals are useful for studying the function and/or activity of the polypeptide and for identifying and/or evaluating modulators of polypeptide activity. In addition to particular gene expression and/or polypeptide expression phenotypes, the transgenic animals of the invention can exhibit any of the phenotypes (e.g., processes, disorder symptoms and/or disorders), as are described in the sections above. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

A transgenic animal of the invention can be created by introducing nucleic acid encoding a BCA polypeptide of the invention (or a homologue thereof) into the male pronuclei of a fertilized oocyte, e.g., by microinjection, retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to the transgene to direct expression of the polypeptide of the invention to particular cells. Methods for generating transgenic animals via embryo manipulation and microinjection, particularly animals such as mice, have become conventional in the art (see, e.g., U.S. Pat. Nos. 4,736,866; 4,870,009; 4,873,191; Hogan, 1986, Manipulating the Mouse Embryo, Cold Spring Harbor Laboratory Press, Cold Spring Harbor, N.Y.; Wakayama et al., 1999, Proc Natl Acad Sci. 96:14984-14989). Similar methods are used for production of other transgenic animals. A transgenic founder animal can be identified based upon the presence of the transgene in its genome and/or expression of mRNA encoding the transgene in tissues or cells of the animals. A transgenic founder animal can then be used to breed additional animals carrying the transgene. Moreover, transgenic animals carrying the transgene can further be bred to other transgenic animals carrying other transgenes.

To create an homologous recombinant animal, a vector is prepared which comprises at least a portion of a gene encoding a polypeptide of the invention into which a deletion, addition or substitution has been introduced to thereby alter, e.g., functionally disrupt, the gene. In a preferred embodiment, the vector is designed such that, upon homologous recombination, the endogenous gene is functionally disrupted (i.e., no longer encodes a functional protein; also referred to as a “knock out” vector). Alternatively, the vector can be designed such that, upon homologous recombination, the endogenous gene is mutated or otherwise altered but still encodes functional protein (e.g., the upstream regulatory region can be altered to thereby alter the expression of the endogenous protein). In the homologous recombination vector, the altered portion of the gene is flanked at its 5′ and 3′ ends by additional nucleic acid of the gene to allow for homologous recombination to occur between the exogenous gene carried by the vector and an endogenous gene in an embryonic stem cell. The additional flanking polynucleotides are of sufficient length for successful homologous recombination with the endogenous gene. Typically, several kilobases of flanking DNA (both at the 5′ and 3′ ends) are included in the vector (See, e.g., Thomas and Capecchi, 1987, Cell 51:503 for a description of homologous recombination vectors). The vector is introduced into an embryonic stem cell line (e.g., by electroporation) and cells in which the introduced gene has homologously recombined with the endogenous gene are selected (See, e.g., Li et al., 1992, Cell 69:915). The selected cells are then injected into a blastocyst of an animal (e.g., a mouse) to form aggregation chimeras (see, e.g., Bradley in Teratocarcinomas and Embryonic Stem Cells: A Practical Approach, Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991, Current Opinion in BioTechnology 2:823-829) and in PCT Publication Nos. WO 90/11354, WO 91/01140, WO 92/0968 and WO 93/04169.

In another embodiment, transgenic non-human animals can be produced which contain selected systems which allow for regulated expression of the transgene. One example of such a system is the cre/loxP recombinase system of bacteriophage P1 (see, e.g., Lakso et al., 1992, Proc Natl Acad Sci. 89:6232-6236). Another example of a recombinase system is the FLP recombinase system of Saccharomyces cerevisiae (O'Gorman et al., 1991, Science 251:1351-1355. If a cre/loxP recombinase system is used to regulate expression of the transgene, animals comprising transgenes encoding both the Cre recombinase and a selected protein are required. Such animals can be provided through the construction of “double” transgenic animals, e.g., by mating two transgenic animals, one comprising a transgene encoding a selected protein and the other comprising a transgene encoding a recombinase.

Clones of the non-human transgenic animals described herein can also be produced, for example, according to the methods described in Wilmut et al., 1997, Nature 385:810-813 and PCT Publication Nos. WO 97/07668 and WO 97/07669.

### 5.4. Antibodies to BCA Polypeptides.

An isolated polypeptide of the invention, or a fragment thereof, can be used as an immunogen to generate antibodies using standard techniques for polyclonal and monoclonal antibody preparation. The full-length polypeptide or protein can be used or, alternatively, the invention provides antigenic peptide fragments for use as immunogens. In one embodiment, the antigenic peptide of a protein of the invention comprises at least 8 (preferably 10, 15, 20, or 30) consecutive amino acid residues of the amino acid sequence of SEQ ID NO:2, 4, 6, 8, 10, 12 or 14, and encompasses an epitope of the protein such that an antibody raised against the peptide forms a specific immune complex with the protein.

Exemplary amino acid sequences of BCA polypeptides that can be used to generate antibodies against BCA genes include CMEPVDAALLSSYETN (SEQ ID NO.:25)(i.e., peptide C14N derived from BCA1), NRSNDSQLNDRWTF (SEQ ID NO.:26)(i.e., peptide N13F derived from BCA2), and AVDSGQSVDLVFPV (SEQ ID NO.:27)(i.e., peptide A14V derived from BCA3). In one embodiment, the immunogenic BCA polypeptide is conjugated to keyhole limpet hemocyanin (“KLH”) and injected into rabbits. Rabbit IgG polyclonal antibodies can purified, for example, on a peptide affinity column.

In one embodiment, the invention provides substantially purified antibodies or fragments thereof, including human or non-human antibodies or fragments thereof, which antibodies. or fragments specifically bind to a polypeptide of the invention comprising an amino acid sequence selected from the group consisting of: the amino acid sequence of SEQ ID NO:2, 4, 6, 8, 10, 12 or 14; a fragment of at least 8 contiguous amino acid residues of the amino acid sequence of SEQ ID NO:2, 4, 6, 8, 10, 12 or 14; an amino acid sequence which is at least 95% identical to the amino acid sequence of SEQ ID NO:2, 4, 6, 8, 10, 12 or 14, wherein the percent identity is determined using the ALIGN program of the GCG software package with a PAM120 weight residue table, a gap length penalty of 12, and a gap penalty of 4; a fragment of at least 15 contiguous amino acid residues of the amino acid sequence of SEQ ID NO:2, 4, 6, 8, 10, 12 or 14; an amino acid sequence which is at least 95% identical to the amino acid sequence of SEQ ID NO:2, 4, 6, 8, 10, 12 or 14, wherein the percent identity is determined using the ALIGN program of the GCG software package with a PAM120 weight residue table, a gap length penalty of 12, and a gap penalty of 4; and an amino acid sequence which is encoded by a polynucleotide which hybridizes to the polynucleotide consisting of SEQ ID NOS:1, 3 (or GenBank Accession No. AW225336), 5 (or GenBank Accession No. AW225337), 7 (or GenBank Accession No. AW225341), 9 (or GenBank Accession No. AW225337), 11 (or GenBank Accession No. AW225337), or 13 (or GenBank Accession No. AW225340}, under conditions of hybridization of 6×SSC at 45° C. and washing in 0.2×SSC, 0.1% SDS at 65° C. In various embodiments, the substantially purified antibodies of the invention, or fragments thereof, can be human, non-human, chimeric and/or humanized antibodies.

In another embodiment, the invention provides human or non-human antibodies or fragments thereof, which antibodies or fragments specifically bind to a polypeptide comprising an amino acid sequence selected from the group consisting of: the amino acid sequence of SEQ ID NO:2, 4, 6, 8, 10, 12 or 14; a fragment of at least 8 contiguous amino acid residues of the amino acid sequence of SEQ ID NO:2, 4, 6, 8, 10, 12 or 14; an amino acid sequence which is at least 95% identical to the amino acid sequence of SEQ ID NO:2, 4, 6, 8, 10, 12 or 14, wherein the percent identity is determined using the ALIGN program of the GCG software package with a PAM120 weight residue table, a gap length penalty of 12, and a gap penalty of 4; a fragment of at least 15 contiguous amino acid residues of the amino acid sequence of SEQ ID NO:2, 4, 6, 8, 10, 12 or 14; an amino acid sequence which is at least 95% identical to the amino acid sequence of SEQ ID NO:2, 4, 6, 8, 10, 12 or 14, wherein the percent identity is determined using the ALIGN program of the GCG software package with a PAM120 weight residue table, a gap length penalty of 12, and a gap penalty of 4; and an amino acid sequence which is encoded by a polynucleotide which hybridizes to the polynucleotide consisting of SEQ ID NO: 1, 3 (or GenBank Accession No. AW225336), 5 (or GenBank Accession No. AW225339), 7 (or GenBank Accession No. AW225341), 9 (or GenBank Accession No. AW225337), 11 (or GenBank Accession No. AW225337), or 13 (or GenBank Accession No. AW225340}, under conditions of hybridization of 6×SSC at 45° C. and washing in 0.2×SSC, 0.1% SDS at 65° C. Such non-human antibodies can be goat, mouse, sheep, horse, chicken, rabbit, or rat antibodies. Alternatively, the non-human antibodies of the invention can be chimeric and/or humanized antibodies. In addition, the non-human antibodies of the invention can be polyclonal antibodies or monoclonal antibodies.

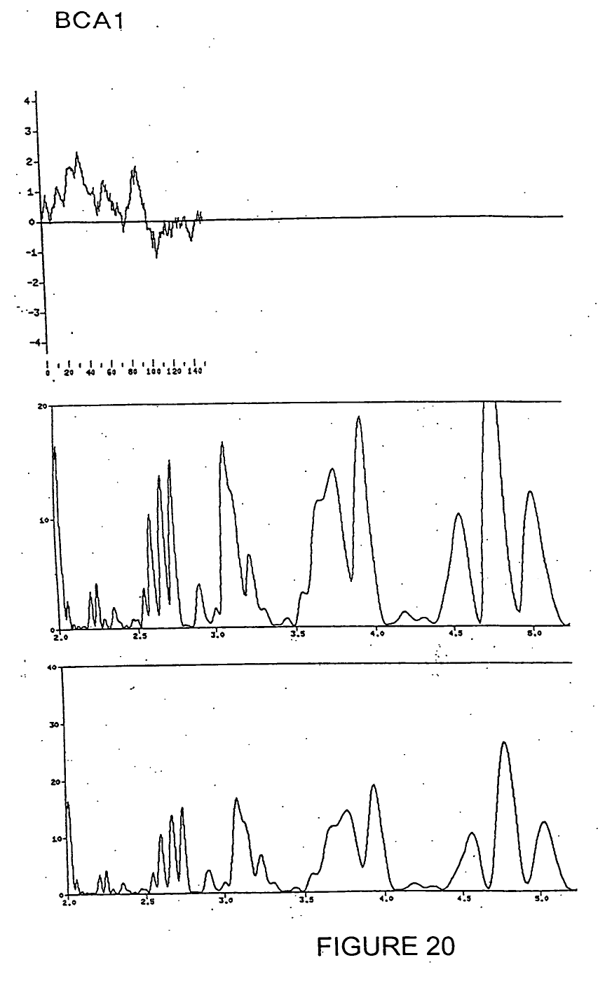

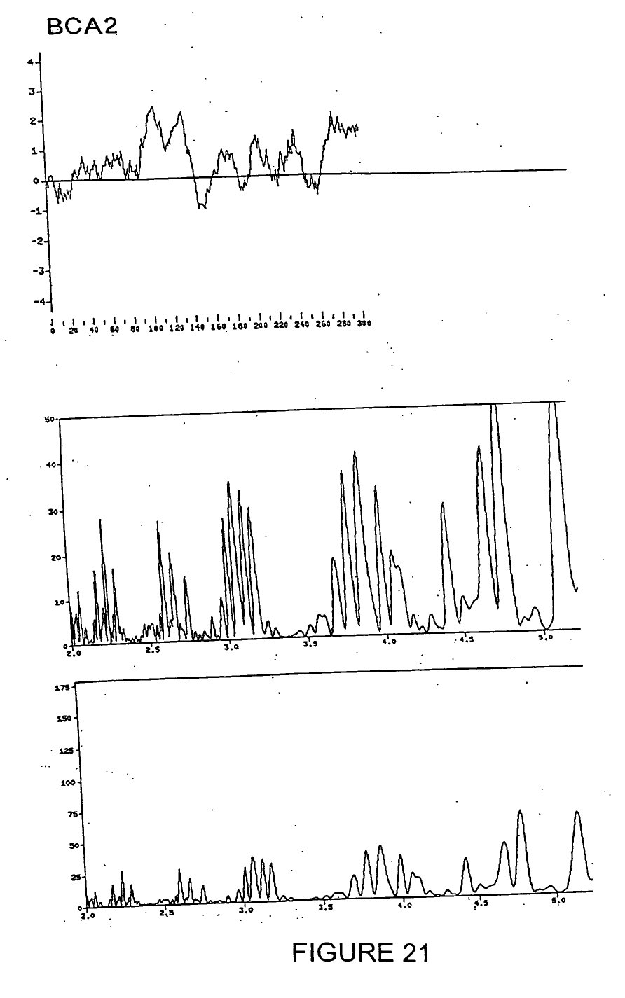

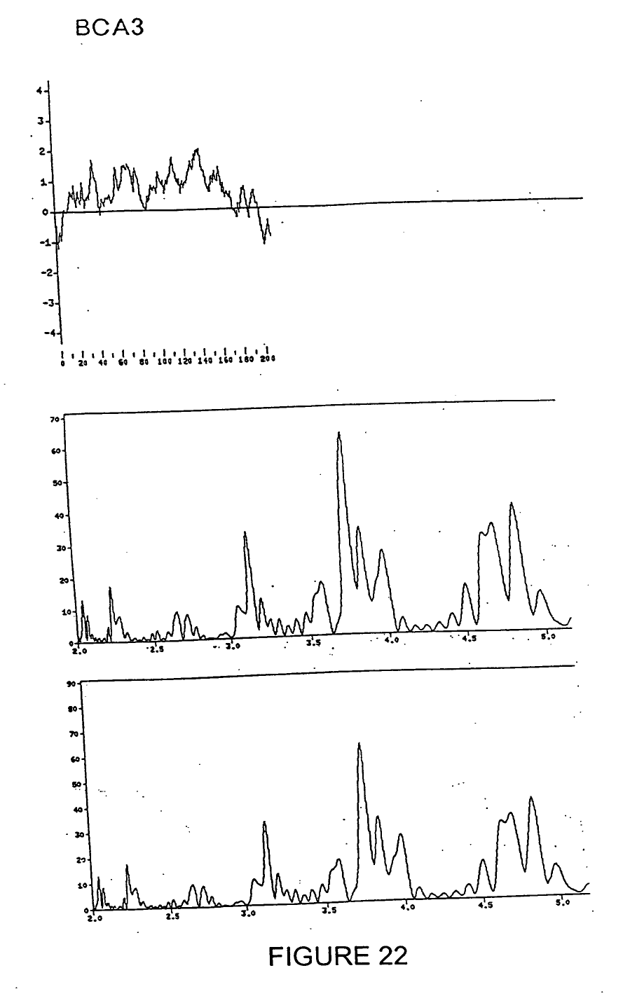

Preferred epitopes encompassed by the antigenic peptide are regions that are located on the surface of the protein, e.g., hydrophilic regions. Hydropathy plots of the proteins of the invention, or similar analyses, can be used to identify hydrophilic regions (FIGS. 20, 21 and 22). In certain embodiments, the polynucleotides of the invention are present as part of polynucleotides comprising nucleic acid sequences that comprise or encode heterologous (e.g., vector, expression vector, or fusion protein) sequences. These nucleotides can then be used to express proteins which can be used as immunogens to generate an immune response, or more particularly, to generate polyclonal or monoclonal antibodies specific to the expressed protein.

An immunogen typically is used to prepare antibodies by immunizing a suitable subject, (e.g., rabbit, goat, mouse or other mammal). An appropriate immunogenic preparation can comprise, for example, recombinantly expressed or chemically synthesized polypeptide. The preparation can further include an adjuvant, such as Freud's complete or incomplete adjuvant, or similar immunostimulatory agent.

Accordingly, another aspect of the invention pertains to antibodies directed against a polypeptide of the invention. The term “antibody” as used herein refers to immunoglobulin molecules and immunologically active portions of immunoglobulin molecules, i.e., molecules that comprise an antigen binding site which specifically binds an antigen, such as a polypeptide of the invention, e.g., an epitope of a polypeptide of the invention. A molecule which specifically binds to a given polypeptide of the invention is a molecule which binds the polypeptide, but does not substantially bind other molecules in a sample, e.g., a biological sample, which naturally comprises the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies. The term “monoclonal antibody” or “monoclonal antibody composition”, as used herein, refers to a population of antibody molecules that comprise only one species of an antigen binding site capable of immunoreacting with a particular epitope.

Polyclonal antibodies can be prepared as described above by immunizing a suitable subject with a BCA polypeptide of the invention as an immunogen. Preferred polyclonal antibody compositions are ones that have been selected for antibodies directed against a polypeptide or polypeptides of the invention. Particularly preferred polyclonal antibody preparations are ones that comprise only antibodies directed against a polypeptide or polypeptides of the invention. Particularly preferred immunogen compositions are those that comprise no other human proteins such as, for example, immunogen compositions made using a non-human host cell for recombinant expression of a polypeptide of the invention. In such a manner, the only human epitope or epitopes recognized by the resulting antibody compositions raised against this immunogen will be present as part of a polypeptide or polypeptides of the invention.

The antibody titer in the immunized subject can be monitored over time by standard techniques, such as with an enzyme linked immunosorbent assay (ELISA) using immobilized polypeptide. If desired, the antibody molecules can be isolated from the mammal (e.g., from the blood) and further purified by well-known techniques, such as protein A chromatography to obtain the IgG fraction. Alternatively, antibodies specific for a BCA polypeptide of the invention can be selected for (e.g., partially purified) or purified by, e.g., affinity chromatography. For example, a recombinantly expressed and purified (or partially purified) protein of the invention is produced as described herein, and covalently or non-covalently coupled to a solid support such as, for example, a chromatography column. The column can then be used to affinity purify antibodies specific for the proteins of the invention from a sample comprising antibodies directed against a large number of different epitopes, thereby generating a substantially purified antibody composition, i.e., one that is substantially free of contaminating antibodies. By a substantially purified antibody composition is meant, in this context, that the antibody sample comprises at most only 30% (by dry weight) of contaminating antibodies directed against epitopes other than those on the desired protein or polypeptide of the invention, and preferably at most 20%, yet more preferably at most 10%, and most preferably at most 5% (by dry weight) of the sample is contaminating antibodies. A purified antibody composition means that at least 99% of the antibodies in the composition are directed against the desired protein or polypeptide of the invention.

At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975, Nature 256:495-497), the human B cell hybridoma technique (Kozbor et al., 1983, Immunol Today 4:72), the EBV-hybridoma technique (Cole et al., 1985, in Monoclonal Antibodies and Cancer Therapy, Alan R. Liss, Inc., pp. 77-96) or trioma techniques. The technology for producing hybridomas is well known (See, e.g., Current Protocols in Immunology, Coligan et al. (eds.) John Wiley & Sons, Inc., New York, N.Y. (1994)). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phase Antibody System, Catalog No. 27-9400-01; and the Stratagene SurfZAP Phage Display Kit, Catalog No. 240612). Additionally, examples of methods and reagents particularly amenable for use in generating and screening an antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, BioTechnology 9:1370-1372; Hay et al., 1992, Hum Antibod Hybridomas 3:81-85; Huse et al., 1989, Science 246:1275-1281; Griffiths et al., 1993, EMBO J. 12:725-7 [symbol] 4.

Additionally, recombinant antibodies, such as chimeric and humanized monoclonal antibodies, comprising both human and non-human portions, which can be made using standard recombinant DNA techniques, are within the scope of the invention. A chimeric antibody is a molecule in which different portions are derived from different animal species, such as those having a variable region derived from a murine mAb and a human immunoglobulin constant region (see, e.g., Cabilly et al., U.S. Pat. No. 4,816,567; and Boss et al., U.S. Pat. No. 4,816,397, which are incorporated herein by reference in their entirety). Humanized antibodies are antibody molecules from non-human species having one or more complementarity determining regions (CDRs) from the non-human species and a framework region from a human immunoglobulin molecule (see, e.g., Queen, U.S. Pat. No. 5,585,089, which is incorporated herein by reference in its entirety). Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al., 1988, Science 240:1041-1043; Liu et al., 1987, Proc Natl Acad Sci. 84:3439-3443; Liu et al., 1987, J. Immunol. 139:3521-3526; Sun et al., 1987, Proc Natl Acad Sci. 84:214-218; Nishimura et al., 1987, Cancer Res. 47:999-1005; Wood et al., 1985, Nature 314:446-449; and Shaw et al., 1988, J. Natl. Cancer Inst. 80:1553-1559; Morrison, 1985, Science 229:1202-1207; Oi et al., 1986, BioTechniques 4:214; U.S. Pat. No. 5,225,539; Jones et al., 1986, Nature 321:552-525; Verhoeyan et al., 1988, Science 239:1534; and Beidler et al., 1988, J. Immunol. 141:4053-4060.

Completely human antibodies are particularly desirable for therapeutic treatment of human patients. Such antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Fremont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., 1994, BioTechnology 12:899-903).

An antibody directed against a BCA polypeptide of the invention (e.g., monoclonal antibody) can be used to isolate the polypeptide by standard techniques, such as affinity chromatography or immunoprecipitation. Moreover, such an antibody can be used to detect the protein (e.g., in a cellular lysate or cell supernatant) in order to evaluate the abundance and pattern of expression of the polypeptide. The antibodies can also be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isotiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologues thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator, a thrombotic agent or an anti-angiogenic agent, e.g., angiostatin or endostatin; or, biological response modifiers such as, for example, lymphokines, interleukin-1 (“IL-1”), interleukin-2 (“IL-2”), interleukin-4 (“IL-4”), interleukin-6 (“IL-6”), interleukin-7 (“IL-7”), granulocyte macrophase colony stimulating factor (“GM-CSF”), granulocyte colony stimulating factor (“G-CSF”), interleukin-10 (“IL-10”), interleukin-12 (“IL-12”), interleukin-15 (“IL-15”), interferon-γ (“IFN-γ”), interferon-α (“IFN-α”), or other immune factors or growth factors.

Techniques for conjugating such therapeutic moiety to antibodies are well known (see, e.g., Amon et al., “Monoclonal Antibodies For Immunotargeting Of Drugs In Cancer Therapy”, in Monoclonal Antibodies And Cancer Therapy, Reisfeld et al. (eds.), Alan R. Liss, Inc. (1985) pp. 243-256; Hellstrom et al., “Antibodies For Drug Delivery” in Controlled Drug Delivery (2nd Ed.), Robinson et al. (eds.) Marcel Dekker, Inc. (1987) pp. 623-653; Thorpe, “Antibody Carriers Of Cytotoxic Agents In Cancer Therapy: A Review”, in Monoclonal Antibodies '84: Biological And Clinical Applications, Pinchera et al. (eds.) (1985) pp. 475-506; “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), Academic Press (1985) pp. 303-316; Thorpe et al., 1992, “The Preparation And Cytotoxic Properties Of Antibody-Toxin Conjugates”, Immunol Rev. 62:119-158).

Alternatively, an antibody can be conjugated to a second antibody to form an antibody heteroconjugate as described in U.S. Pat. No. 4,676,980.

An antibody with or without a therapeutic moiety conjugated to it can be used as a therapeutic that is administered alone or in combination with chemotherapeutic agents.

Alternatively, an antibody of the invention can be conjugated to a second antibody to form an “antibody heteroconjugate” as described in U.S. Pat. No. 4,676,980 or alternatively, two antibodies can be conjugated to each other to create a bispecific heteromers, or an “antibody heteropolymer” as described in U.S. Pat. Nos. 5,470,570 and 5,487,890.

An antibody with or without a therapeutic moiety conjugated to it can be used as a therapeutic that is administered alone or in combination with cytotoxic factor(s) and/or cytokine(s).

In still a further aspect, the invention provides monoclonal antibodies or fragments thereof, which antibodies or fragments specifically bind to a polypeptide of the invention comprising an amino acid sequence selected from the group consisting of: the amino acid sequence of SEQ ID NO:2, 4, 6, 8, 10, 12 or 14; a fragment of at least 15 contiguous amino acid residues of the amino acid sequence of SEQ ID NO:2, 4, 6, 8, 10, 12 or 14; an amino acid sequence which is at least 95% identical to the amino acid sequence of SEQ ID NO:2, 4, 6, 8, 10, 12 or 14, wherein the percent identity is determined using the ALIGN program of the GCG software package with a PAM 120 weight residue table, a gap length penalty of 12, and a gap penalty of 4; and an amino acid sequence which is encoded by a polynucleotide which hybridizes to the polynucleotide consisting of NO:1, 3 (or GenBank Accession No. AW225336), 5 (or GenBank Accession No. AW225337), 7 (or GenBank Accession No. AW225341), 9 (or GenBank Accession No. AW225337), 11 (or GenBank Accession No. AW225337), or 13 (or GenBank Accession No. AW225340}, under conditions of hybridization of 6×SSC at 45° C. and washing in 0.2×SSC, 0.1% SDS at 65° C. The monoclonal antibodies can be human, humanized, chimeric and/or non-human antibodies.

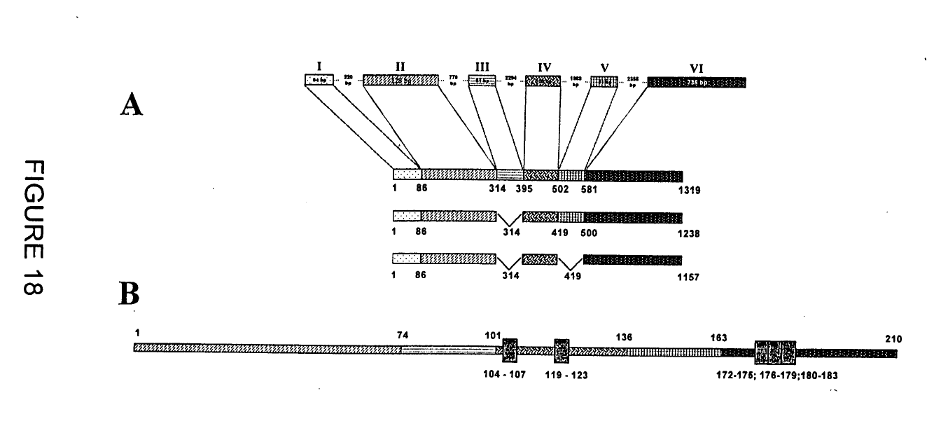

The substantially purified antibodies or fragments thereof can specifically bind to a signal peptide, a secreted sequence, an extracellular domain, a transmembrane or a cytoplasmic domain cytoplasmic membrane of a polypeptide of the invention. In a particularly preferred embodiment, the substantially purified antibodies or fragments thereof, the non-human antibodies or fragments thereof, and/or the monoclonal antibodies or fragments thereof, of the invention specifically bind to a secreted sequence, or alternatively, to an extracellular domain of the amino acid sequence of the invention. Examples of preferred epitopes, i.e., epitopes in extracellular domains of polypeptides of the invention, can be identified using hydropathy plots (FIGS. 18, 19, and 20).

Any of the antibodies of the invention can be conjugated to a therapeutic moiety or to a detectable substance. Non-limiting examples of detectable substances that can be conjugated to the antibodies of the invention are an enzyme, a prosthetic group, a fluorescent material, a luminescent material, a bioluminescent material, and a radioactive material.

Still another aspect of the invention is a method of making an antibody that specifically recognizes a BCA polypeptide of the invention, the method comprising immunizing a mammal with a BCA polypeptide. The BCA polypeptide used as an immunogen comprises an amino acid sequence selected from the group consisting of: the amino acid sequence of any one of SEQ ID NO:2, 4, 6, 8, 10, 12 or 14; a fragment of at least 15 contiguous amino acid residues of the amino acid sequence of any one of SEQ ID NO:2, 4, 6, 8, 10, 12 or 14 an amino acid sequence which is at least 95% identical to the amino acid sequence of any one of SEQ ID NO:2, 4, 6, 8, 10, 12 or 14 wherein the percent identity is determined using the ALIGN program of the GCG software package with a PAM120 weight residue table, a gap length penalty of 12, and a gap penalty of 4; and an amino acid sequence which is encoded by a polynucleotide which hybridizes to the polynucleotide consisting of any one of SEQ ID NO: 1, 3 (or GenBank Accession No. AW225336), 5 (or GenBank Accession No. AW225339), 7 (or GenBank Accession No. AW225341), 9 (or GenBank Accession No. AW225337), 11 (or GenBank Accession No. AW225337), or 13 (or GenBank Accession No. AW225340), or a complement thereof, under conditions of hybridization of 6×SSC at 45° C. and washing in 0.2×SSC, 0.1% SDS at 65° C. After immunization, a sample is collected from the mammal that comprises an antibody that specifically recognizes the immunogen. Preferably, the polypeptide is recombinantly produced using a non-human host cell. Optionally, the antibodies can be further purified from the sample using techniques well known to those of skill in the art. The method can further comprise producing a monoclonal antibody-producing cell from the cells of the mammal. Optionally, antibodies are collected from the antibody-producing cell.

In further embodiments, the invention provides an antibody that immunospecifically binds to a human BCA polypeptide when bound to a binding partner, wherein said polypeptide is selected from the group consisting of BCA1, BCA2, BCA3, BCA4, BCA5, BCA6, and BCA7. In yet another embodiment, the invention provides an antibody that immunospecifically binds to a human BCA polypeptide when bound to a binding partner; wherein said antibody does not bind to said polypeptide when not bound to said binding partner; and wherein said polypeptide is selected from the group consisting of BCA1, BCA2, BCA3, BCA4, BCA5, BCA6, and BCA7.

### 5.5. Functional Analysis of the BCA Genes.

5.5.1. Phosphorylation Assays.

The nature and position of phosphorylation of the proteins encoded by BCA1-7 can be determined. For example, a construct, BCA3/pCMV-tag2B which expresses an N-terminal FLAG-tagged BCA3 protein can be used in in vitro transcription/translation reactions to generate a ³⁵S-Met labeled product. The size of this product could be confirmed by autoradiography or immunoblot analysis of the non-radioactive reaction mixtures using anti-FLAG M2 monoclonal antibodies. This construct can be used to determine the phosphorylation of BCA3 by standard methods to assay phosphorylation.

5.5.2. Identification of Protein Binding Partners

Two-hybrid screening is well known in the art and can be performed to identify binding partners of the BCA1-7 gene products. Accordingly, the yeast two-hybrid cloning method can be used to identify and isolate cDNAs encoding potential protein partners of a BCA polypeptide of the invention. For example, cDNAs encoding binding partners of a BCA1 variant have been identified (see Table 1 and Examples below).

### 5.6. Uses and Methods of the Invention

The polynucleotides, proteins, protein homologues, and antibodies described herein can be used in one or more of the following methods: a) screening assays; b) detection assays (e.g., chromosomal mapping, tissue typing, forensic biology); c) predictive medicine (e.g., diagnostic assays, prognostic assays, monitoring clinical trials, and pharmacogenomics); and d) methods of treatment (e.g., therapeutic and prophylactic). For example, polypeptides of the invention can to used to (i) modulate cellular proliferation; (ii) modulate cellular differentiation; and/or (iii) modulate cellular adhesion. The isolated polynucleotides of the invention can be used to express proteins (e.g., via a recombinant expression vector in a host cell in gene therapy applications), to detect mRNA (e.g., in a biological sample) or a genetic lesion, and to modulate activity of a polypeptide of the invention. In addition, the BCA polynucleotides or BCA polypeptides of the invention can be used to screen drugs o: compounds which bind to and/or modulate activity or expression of a polypeptide of the invention as well as to treat disorders characterized by insufficient or excessive production of a protein of the invention or production of a form of a protein of the invention which has decreased or aberrant activity compared to the wild type protein. In addition, the antibodies of the invention can be used to detect, isolate, and modulate activity of a protein of the invention.

This invention further pertains to use of agents identified by the above-described screening assays (e.g., binding partners, agonists, antagonists), complexes comprising such agents and a BCA polynucleotide and/or BCA polypeptide, and inhibitors of the interactions of such agents with a BCA polynucleotide and/or BCA polypeptide.

5.6.1. Expression Pattern of Breast Cancer-Associated Genes in Human Tissues/Organs.

Cellular and subcellular localization of BCA1-7 expression can be determined by various methods known in the art, such as but not limited to GFP-fusion protein expression. In one embodiment, each construct would consist of GFP and a BCA polynucleotide. The localization of the GFP-BCA fusion protein could be determined by, for example, fluorescence microscopy.

Differential expression of BCA1-7 at different stages of disease progression can be determined by immunohistochemistry and transcriptome (genechip) methodologies.

5.6.2. Screening Assays.

5.6.2.1. Assays for the Identification of Modulators of a BCA Nucleic Acid or Protein.

The present invention also provides isolated BCA polynucleotides, or variants thereof, as probes that can be used to screen for DNA-binding proteins including, but not limited to, proteins that affect DNA conformation or modulate transcriptional activity (e.g., enhancers, transcription factors). In another embodiment, such probes can be used to screen for RNA-binding factors, including but not limited to proteins, steroid hormones, or other small molecules. In yet another embodiment, such probes can be used to detect and identify molecules that bind or affect the pharmacokinetics or activity (e.g., enzymatic activity) of a polypeptide of the invention.

In one embodiment, a screening assay of the invention can identify a test compound that is useful for increasing or decreasing the translation of a BCA mRNA, for example, by binding to one or more regulatory elements in the 5′ untranslated region, the 3′ untranslated region, or the coding region of the mRNA. Compounds that bind to mRNA can, inter alia, increase or decrease the rate of mRNA processing, alter its transport through the cell, prevent or enhance binding of the mRNA to ribosomes, suppressor proteins or enhancer proteins, or alter mRNA stability. Accordingly, compounds that increase or decrease mRNA translation can be used to treat or prevent disease. For example, diseases such as cancer, associated with overproduction of proteins, such as Ras, can be treated or prevented by decreasing translation of the mRNA that codes for the overproduced protein, thus inhibiting production of the protein. Conversely, the symptoms of diseases associated with decreased protein function, such as hemophelia, may be treated by increasing translation of mRNA coding for the protein whose function is decreased, e.g., factor IX in some forms of hemophilia.

Accordingly, in one embodiment, a compound identified by a screening assay of the invention inhibits the production of a BCA protein. In a further embodiment, the compound inhibits the translation of a BCA mRNA.

The invention provides a method for identifying modulators, i.e., candidate or test compounds or agents (e.g., peptides, peptidomimetics, small molecules or other drugs) which bind to polypeptide of the invention or have a stimulatory or inhibitory effect on, for example, expression or activity of a polypeptide of the invention.

In one embodiment, the invention provides assays for screening candidate or test compounds which bind to or modulate the activity of the membrane-bound form of a polypeptide of the invention or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al., 1993, Proc Natl Acad Sci. 90:6909; Erb et al., 1994, Proc Natl Acad Sci. 91:11422; Zuckermann et al., 1994, J. Med Chem. 37:2678; Cho et al., 1993, Science 261:1303; Carrell et al., 1994, Angew Chem Int Ed Encl. 33:2059; Carell et al., 1994, Angew Chem Int Ed Encl. 33:2061; and Gallop et al., 1994, J. Med. Chem. 37:1233.

Libraries of compounds may be presented in solution (e.g., Houghten, 1992, BioTechniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, Proc Natl Acad Sci. 89:1865-1869) orphage (Scott and Smith, 1990, Science 249:386-390; Dev [symbol] n, 1990, Science 249:404-406; Cwirla et al., 1990, Proc Natl Acad Sci. 87:6378-6382; and Felici, 1991, J. Mol. Biol. 222:301-310).

In one embodiment, an assay is a cell-based assay in which a cell which expresses a membrane-bound form of a polypeptide of the invention, or a biologically active portion thereof, on the cell surface is contacted with a test compound and the ability of the test compound to bind to the polypeptide determined. The cell, for example, can be a yeast cell or a cell of mammalian origin. Determining the ability of the test compound to bind to the polypeptide can be accomplished, for example, by coupling the test compound with a radioisotope or enzymatic label such that binding of the test compound to the polypeptide or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radio-emission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In a preferred embodiment, the assay comprises contacting a cell which expresses a membrane-bound form of a polypeptide of the invention, or a biologically active portion thereof, on the cell surface with a known compound which binds the polypeptide to form an assay mixture, contacting the assay mixture with a test compound, and determining the ability of the test Compound to interact with the polypeptide, wherein determining the ability of the test compound to interact with the polypeptide comprises determining the ability of the test compound to preferentially bind to the polypeptide or a biologically active portion thereof as compared to the known compound.

In another embodiment, an assay is a cell-based assay comprising contacting a cell expressing a membrane-bound form of a polypeptide of the invention, or a biologically active portion thereof, on the cell surface with a test compound and determining the ability of the test compound to modulate (e.g., stimulate or inhibit) the activity of the polypeptide or biologically active portion thereof. Determining the ability of the test compound to modulate the activity of the polypeptide or a biologically active-portion thereof can be accomplished, for example, by determining the ability of the polypeptide protein to bind to or interact with a target molecule.

Determining the ability of a polypeptide of the invention to bind to or interact with a target molecule can be accomplished by one of the methods described above for determining direct binding. As used herein, a “target molecule” is a molecule with which a selected polypeptide (e.g., a BCA polypeptide of the invention) binds or interacts with in nature, for example, a molecule on the surface of a cell which expresses the selected protein, a molecule on the surface of a second cell, a molecule in the extracellular milieu, a molecule associated with the internal surface of a cell membrane or a cytoplasmic molecule. A target molecule can be a polypeptide of the invention or some other polypeptide or protein. For example, a target molecule can be a component of a signal transduction pathway which facilitates transduction of an extracellular signal (e.g., a signal generated by binding of a compound to a polypeptide of the invention) through the cell membrane and into the cell or a second intercellular protein which has catalytic activity or a protein which facilitates the association of downstream signaling molecules with a polypeptide of the invention. Determining the ability of a polypeptide of the invention to bind to or interact with a target molecule can be accomplished by determining the activity of the target molecule. For example, the activity of the target molecule can be determined by detecting induction of a cellular second messenger of the target (e.g., intracellular Ca²⁺, diacylglycerol, IP3, etc.), detecting catalytic/enzymatic activity of the target on an appropriate substrate, detecting the induction of a reporter gene (e.g., a regulatory element that is responsive to a polypeptide of the invention operably linked to a nucleic acid encoding a detectable marker, e.g., luciferase), or detecting a cellular response, for example, cellular differentiation, or cell proliferation.

In yet another embodiment, an assay of the present invention is a cell-free assay comprising contacting a BCA polypeptide of the invention or biologically active portion thereof with a test compound and determining the ability of the test compound to bind to the polypeptide or biologically active portion thereof. Binding of the test compound to the polypeptide can be determined either directly or indirectly as described above. In a preferred embodiment, the assay includes contacting the polypeptide of the invention or biologically active portion thereof with a known compound which binds the polypeptide to form an assay mixture, contacting the assay mixture with a test compound, and determining the ability of the test compound to interact with the polypeptide, wherein determining the ability of the test compound to interact with the polypeptide comprises determining the ability of the test compound to preferentially bind to the polypeptide or biologically active portion thereof as compared to the known compound.

In another embodiment, an assay is a cell-free assay comprising contacting a BCA polypeptide of the invention or biologically active portion thereof with a test compound and determining the ability of the test compound to modulate (e.g., stimulate or inhibit) the activity of the polypeptide or biologically active portion thereof. Determining the ability of the test compound to modulate the activity of the polypeptide can be accomplished. for example, by determining the ability of the polypeptide to bind to a target molecule by one of the methods described above for determining direct binding. In an alternative embodiment, determining the ability of the test compound to modulate the activity of the polypeptide can be accomplished by determining the ability of the polypeptide of the invention to further modulate the target molecule. For example, the catalytic/enzymatic activity of the target molecule on an appropriate substrate can be determined as previously described.

In yet another embodiment, the cell-free assay comprises contacting a BCA polypeptide of the invention or biologically active portion thereof with a known compound which binds the polypeptide to form an assay mixture, contacting the assay mixture with a test compound, and determining the ability of the test compound to interact with the polypeptide, wherein determining the ability of the test compound to interact with the polypeptide comprises determining the ability of the polypeptide to preferentially bind to or modulate the activity of a target molecule.

The cell-free assays of the present invention are amenable to use of both a soluble form or the membrane-bound form of a polypeptide of the invention. In the case of cell-free assays comprising the membrane-bound form of the polypeptide, it may be desirable to utilize a solubilizing agent such that the membrane-bound form of the polypeptide is maintained in solution. Examples of such solubilizing agents include non-ionic detergents such as n-octylglucoside, n-dodecylglucoside, n-octylmaltoside, octanoyl-N-methylglucamide, decanoyl-N-methylglucamide, Triton X-100, Triton X-114, Thesit, Isotridecypoly(ethylene glycol ether)n, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In more than one embodiment of the above assay methods of the present invention, it may be desirable to immobilize either the BCA polypeptide of the invention or its target molecule to facilitate separation of complexed from uncomplexed forms of one or both of the proteins, as well as to accommodate automation of the assay. Binding of a test compound to the polypeptide, or interaction of the polypeptide with a target molecule in the presence and absence of a candidate compound, can be accomplished in any vessel suitable for comprising the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase fusion proteins or glutathione-S-transferase fusion proteins can be adsorbed onto glutathione sepharose beads (Sigma Chemical; St. Louis, Mo.) or glutathione derivatized microtiter plates, which are then combined with the test compound or the test compound and either the non-adsorbed target protein or A polypeptide of the invention, and the mixture incubated under conditions conducive to complex formation (e.g., at physiological conditions for salt and pH). Following incubation, the beads or microtiter plate wells are washed to remove any unbound components and complex formation is measured either directly or indirectly, for example, as described above. Alternatively, the complexes can be dissociated from the matrix, and the level of binding or activity of the polypeptide of the invention can be determined using standard techniques.

Other techniques for immobilizing proteins on matrices can also be used in the screening assays of the invention. For example, either the polypeptide of the invention or its target molecule can be immobilized utilizing conjugation of biotin and streptavidin. Biotinylated polypeptide of the invention or target molecules can be prepared from biotin-NHS (N-hydroxy-succinimide) using techniques well known in the art (e.g., biotinylation kit, Pierce Chemicals; Rockford, Ill.), and immobilized in the wells of streptavidin-coated 96 well plates (Pierce Chemical). Alternatively, antibodies reactive with the polypeptide of the invention or target molecules but which do not interfere with binding of the polypeptide of the invention to its target molecule can be derivatized to the wells of the plate, and unbound target or polypeptide of the invention trapped in the wells by antibody conjugation. Methods for detecting such complexes, in addition to those described above for the GST-immobilized complexes, include immunodetection of complexes using antibodies reactive with the polypeptide of the invention or target molecule, as well as enzyme-linked assays which rely on detecting an enzymatic activity associated with the polypeptide of the invention or target molecule.

In another embodiment, modulators of expression of a BCA polypeptide of the invention are identified in a method in which a cell is contacted with a candidate compound and the expression of the selected mRNA or protein (i.e., the mRNA or protein corresponding to a polypeptide or nucleic acid of the invention) in the cell is determined. The level of expression of the selected mRNA or protein in the presence of the candidate compound is compared to the level of expression of the selected mRNA or protein in the absence of the candidate compound. The candidate compound can then be identified as a modulator of expression of the polypeptide of the invention based on this comparison. For example, when expression of the selected mRNA or protein is greater (statistically significantly greater) in the presence of the candidate compound than in its absence, the candidate compound is identified as a stimulator of the selected mRNA or protein expression. Alternatively, when expression of the selected mRNA or protein is less (statistically significantly less) in the presence of the candidate compound than in its absence, the candidate compound is identified as an inhibitor of the selected mRNA or protein expression. The level of the selected mRNA or protein expression in the cells can be determined by methods described herein.

In yet another aspect of the invention, a BCA polypeptide of the inventions can be used as “bait proteins” in a two-hybrid assay or three hybrid assay (see, e.g., U.S. Pat. No. 5,283,317; Zervos et al., 1993, Cell 72:223-232; Madura et al., 1993, J Biol Chem. 268:12046-12054; Bartel et al., 1993, BioTechniques 14:920-924; Iwabuchi et al., 1993, Oncogene 8:1693-1696; and PCT Publication No. WO 94/10300), to identify other proteins, which bind to or interact with the polypeptide of the invention and modulate activity of the polypeptide of the invention. Such binding proteins are also likely to be involved in the propagation of signals by the polypeptide of the inventions as, for example, upstream or downstream elements of a signaling pathway involving the polypeptide of the invention.

The invention also provides a method for screening for compounds (e.g., potentially useful drugs) that bind a BCA polynucleotide or polypeptide. In one embodiment, test compounds are assayed for binding to a BCA polynucleotide or polypeptide. In another embodiment, test compounds are assayed for binding to a complex comprising a BCA polynucleotide (e.g., transcriptional complex) or a BCA polypeptide (hetero- or homo-dimer or multimer). In a further embodiment, test compounds are assayed for binding to a BCA polypeptide when bound to a second, different polypeptide. In a further embodiment, test compounds are assayed for binding to a BCA1 polypeptide when bound to a Smurf2, AIP4 and/or polypeptide encoded from a cDNA listed in Table 1.

In one embodiment, the invention also provides a method for screening for compounds that bind to a complex comprising a BCA polynucleotide or BCA polypeptide, wherein the compound binds to a BCA polynucleotide or BCA polypeptide when bound to its binding partner, and does not bind to the BCA polynucleotide or BCA polypeptide when not bound to the binding partner. In a specific embodiment, the compound binds to a BCA1 polypeptide when bound to a binding partner, and does not bind to the BCA1 polypeptide when not bound to the binding partner.

The invention also provides a method for screening for compounds (e.g., potentially useful drugs) that inhibit the binding of a BCA polynucleotide or polypeptide to an analyte, target molecule or binding partner. In one embodiment, test compounds are assayed to prevent formation of complexes comprising a BCA polynucleotide (e.g., transcriptional complex) or a BCA polypeptide (hetero- or homo-dimer or multimer). In a further embodiment, test compounds are assayed for ability to inhibit binding of a BCA polypeptide to a second, different polypeptide. In a further embodiment, test compounds are assayed for ability to inhibit binding of a BCA1 polypeptide to a Smurf2, AIP4 and/or polypeptide encoded from a cDNA listed in Table 1.

In particular embodiments, the test compounds are assayed for the ability to interfere with existing complexes or existing interactions of a BCA polynucleotide or polypeptide with another compound. In other embodiments, the test compound is incubated first with the BCA polynucleotide or polypeptide, prior to addition of the analyte, target molecule or binding partner, after which the ability to inhibit binding is assayed. In yet other embodiments, the test compound is incubated first with the analyte, target molecule or binding partner, prior to addition of the BCA polynucleotide or polypeptide, after which the ability to inhibit binding is assayed.

Accordingly, in one embodiment, the present invention provides a method for identifying an analyte that disrupts a complex comprising a BCA polynucleotide or BCA polypeptide comprising the steps of contacting the complex with the analyte, and detecting dissociation of the complex. In another embodiment, the invention provides a method for identifying an analyte that inhibits formation of a complex comprising a BCA polynucleotide or BCA polypeptide comprising the steps of contacting one or more members of the complex with the analyte prior to complex formation, and then incubating the members of the complex under conditions that normally would allow complex formation, followed by detecting inhibition of complex formation.

In one embodiment, the invention provides an antibody that immunospecifically binds to a human BCA polypeptide, wherein the polypeptide is selected from the group consisting of BCA1, BCA2, BCA3, BCA4, BCA5, BCA6, and BCA7. In another embodiment, the antibody immunospecifically binds to a human BCA polypeptide when bound to a binding partner, wherein the polypeptide is selected from the group consisting of BCA1, BCA2, BCA3, BCA4, BCA5, BCA6, and BCA7. In another embodiment, the antibody immunospecifically binds to a human BCA polypeptide when bound to a binding partner; wherein the antibody does not bind to the polypeptide when not bound to the binding partner; and wherein the polypeptide is selected from the group consisting of BCA1, BCA2, BCA3, BCA4, BCA5, BCA6, and BCA7.

The present invention also provides methods for identifying an analyte that binds a BCA polypeptide. In one embodiment the method comprises the steps of contacting the BCA polypeptide with an analyte under conditions that allow the BCA polypeptide to be bound by the analyte, and detecting binding of the BCA polypeptide to the analyte, wherein the BCA polypeptide is selected from the group consisting of a BCA1, BCA2, BCA3, BCA4, BCA5, BCA6, and BCA7 polypeptide, and wherein detection of binding indicates presence of an analyte that binds the BCA polypeptide.

In another embodiment, the method comprises the steps of contacting the BCA polypeptide with a positionally addressable array comprising a plurality of proteins, with each protein being at a different position on a solid support, and detecting binding of the BCA polypeptide to a protein on the array, wherein the BCA polypeptide is selected from the group consisting of a BCA1, BCA2, BCA3, BCA4, BCA5, BCA6, and BCA7 polypeptide, and wherein detection of binding indicates presence of a protein that binds the BCA polypeptide. Such arrays (e.g., protein arrays) are known in the art.

The present invention also provides a method for screening for a compound that binds to a complex comprising a BCA polynucleotide or BCA polypeptide, wherein the compound binds to a BCA polynucleotide or BCA polypeptide when bound to its binding partner, and does not bind to the BCA polynucleotide or BCA polypeptide when not bound to the binding partner. Such screening assays can be useful to identify novel binding partners that modulate a BCA polynucleotide or BCA polypeptide such that the compound detects complex formation with heterologous compounds. The invention encompasses any such novel binding partners.

The present invention also provides a method for screening for proteins that bind a BCA polypeptide and regulate ubiquitination. The present invention also provides screening assays can be performed to detect compounds that affect the binding of a BCA polypeptide to a protein involved in the ubiquitination pathway. In a specific embodiment, the BCA polypeptide binds to a protein that regulates ubiquitination of a tumor suppressor (e.g., Syk).

Such assays can be used to identify the domains of BCA polypeptides that are required for binding to ubiquitin-regulating proteins. Polynucleotides encoding these domains could be used as bait in protein-protein interaction screening assays such as two-hybrid analysis. In particular embodiments, mutations in BCA polynucleotides which enhance or inhibit the binding of ubiquitin-related proteins to BCA polypeptides could be identified.

Monitoring the influence of agents (e.g., drugs, compounds) on the expression or activity of a polypeptide of the invention (e.g., the ability to modulate aberrant cell proliferation chemotaxis, and/or differentiation) can be applied not only in basic drug screening, but also in clinical trials. For example, the effectiveness of an agent, as determined by a screening assay as described herein, to increase gene expression, protein levels or protein activity, can be monitored in clinical trials of subjects exhibiting decreased gene expression, protein levels, or protein activity. Alternatively, the effectiveness of an agent, as determined by a screening assay, to decrease gene expression, protein levels or protein activity, can be monitored in clinical trials of subjects exhibiting increased gene expression, protein levels, or protein activity. In such clinical trials, expression or activity of a polypeptide of the invention and preferably, that of other polypeptide that have been implicated in for example, a cellular proliferation disorder, can be used as a marker of the immune responsiveness of a particular cell.

For example, and not by way of limitation, genes, including those of the invention, that are modulated in cells by treatment with an agent (e.g., compound, drug or small molecule) which modulates activity or expression of a polypeptide of the invention (e.g., as identified in a screening assay described herein) can be identified. Thus, to study the effect of agents on cellular proliferation disorders, for example, in a clinical trial, cells can be isolated and RNA prepared and analyzed for the levels of expression of a gene of the invention and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of a gene of the invention or other genes. In this way, the gene expression pattern can serve as a marker, indicative of the physiological response of the cells to the agent. Accordingly, this response state may be determined before, and at various points during, treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of the polypeptide or nucleic acid of the invention in the preadministration sample; (iii) obtaining one or more post-administration samples from the subject; (iv) detecting the level the of the polypeptide or nucleic acid of the invention in the post-administration samples; (v) comparing the level of the polypeptide or nucleic acid of the invention in the pre-administration sample with the level of the polypeptide or nucleic acid of the invention in the post-administration sample or samples; and (vi) altering the administration of the agent to the subject accordingly. For example, increased administration of the agent may be desirable to increase the expression or activity of the polypeptide to higher levels than detected, i.e., to increase the effectiveness of the agent. Alternatively, decreased administration of the agent may be desirable to decrease expression or activity of the polypeptide to lower levels than detected, i.e., to decrease the effectiveness of the agent.

This invention further pertains to uses of agents identified by the above-described screening assays.

5.6.3. Detection Assays.

Portions or fragments of the cDNA sequences identified herein (and the corresponding complete gene sequences) can be used in numerous ways as polynucleotide reagents. For example, these sequences can be used to: (i) map their respective genes on a chromosome and, thus, locate gene regions associated with genetic disease; (ii) identify an individual from a minute biological sample (tissue typing); and (iii) aid in forensic identification of a biological sample. These applications are described in the subsections below.

5.6.4. Chromosome Mapping.

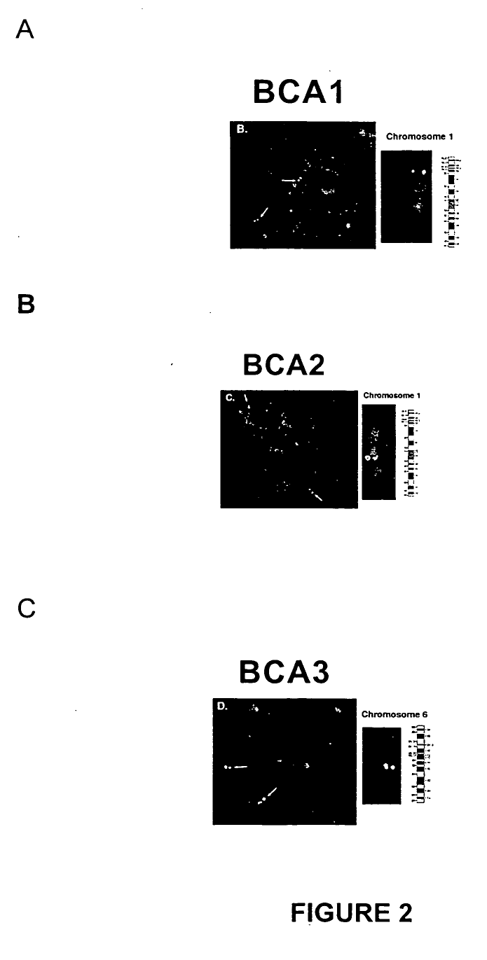

Once the sequence (or a portion of the sequence) of a gene has been isolated, this sequence can be used to map the location of the gene on a chromosome (FIG. 2). Accordingly, BCA polynucleotides described herein or fragments thereof, can be used to map the location of the corresponding genes on a chromosome. The mapping of the sequences to chromosomes is an important first step in correlating these sequences with chromosomal aberrations associated with bcl-related disease.

Briefly, genes can be mapped to chromosomes by preparing PCR primers (preferably 15-25 bp in length) from the sequence of a gene of the invention. Computer analysis of the sequence of a gene of the invention can be used to rapidly select primers that do not span more than one exon in the genomic DNA, thus complicating the amplification process. These primers can then be used for PCR screening of somatic cell hybrids containing individual human chromosomes. Only those hybrids comprising the human gene corresponding to the gene sequences will yield an amplified fragment. For a review of this technique, see D'Eustachio et al. (1983, Science 220:919-924).

PCR mapping of somatic cell hybrids is a rapid procedure for assigning a particular sequence to a particular chromosome. Three or more sequences can be assigned per day using a single thermal cycler. Using the polynucleotides of the invention to design oligonucleotide primers, sublocalization can be achieved with panels of fragments from specific chromosomes. Other mapping strategies which can similarly be used to map a gene to its chromosome include in situ hybridization (described in Fan et al., 1990, Proc Natl Acad Sci. 87:6223-6227), pre-screening with labeled flow-sorted chromosomes (CITE), and pre-selection by hybridization to chromosome specific cDNA libraries. Fluorescence in situ hybridization (FISH) of a DNA sequence to a metaphase chromosomal spread can further be used to provide a precise chromosomal location in one step. For a review of this technique, see Verma et al., (Human Chromosomes: A Manual of Basic Techniques (Pergamon Press, New York, 1988)).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available at the Johns Hopkins University Welch Medical Library website). The relationship between genes and disease, mapped to the same chromosomal region, can then be identified through linkage analysis (co-inheritance of physically adjacent genes)(see, e.g., Egeland et al., 1987, Nature 325:783-787).

Moreover, differences in the DNA sequences between individuals affected and unaffected with a disease associated with a gene of the invention can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Furthermore, the polynucleotides disclosed herein can be used to perform searches against “mapping databases”, e.g., BLAST-type search, such that the chromosome position of the gene is identified by sequence homology or identity with known sequence fragments which have been mapped to chromosomes.

In addition, a polypeptide and fragments and sequences thereof and antibodies specific thereto can be used to map the location of the gene encoding the polypeptide on a chromosome. This mapping can be carried out by specifically detecting the presence of the polypeptide in members of a panel of somatic cell hybrids between cells of a first species of animal from which the protein originates and cells from a second species of animal and then determining which somatic cell hybrid(s) expresses the polypeptide and noting the chromosome(s) from the first species of animal that it contains. For examples of this technique, see Pajunen et al., 1988, Cytogenet Cell Genet. 47:3741 and Van Keuren et al., 1986, Hum Genet. 74:34-40. Alternatively, the presence of the polypeptide in the somatic cell hybrids can be determined by assaying an activity or property of the polypeptide, for example, enzymatic activity, as described in Bordelon-Riser et al., 1979, Somatic Cell Genetics 5:597-613 and Owerbach et al., 1978, Proc Natl Acad Sci. 75:5640-5644.

5.6.5. Tissue Typing.

The BCA polynucleotides of the present invention can also be used to identify individuals from minute biological samples. The United States military, for example, is considering the use of restriction fragment length polymorphism (“RFLP”) analysis for identification of its personnel. In this technique, an individual's genomic DNA is digested with one or more restriction enzymes, and probed on a Southern blot to yield unique bands for identification. This method does not suffer from the current limitations of “Dog Tag” which can be lost, switched, or stolen, making positive identification difficult. The sequences of the present invention are useful as additional DNA markers for RFLP analysis (see, e.g., U.S. Pat. No. 5,272,057).

Furthermore, the sequences of the present invention can be used to provide an alternative technique which determines the actual base-by-base DNA sequence of selected portions of an individual's genome. Thus, the polynucleotides described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The polynucleotides of the invention uniquely represent portions of the human genome. Allelic variation occurs to some degree in the coding regions of these sequences, and to a greater degree in the noncoding regions. It is estimated that allelic variation between individual humans occurs with a frequency at about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO: 1, 3 (or GenBank Accession No. AW225336), 5 (or GenBank Accession No. AW225339), 7 (or GenBank Accession No. AW225341), 9 (or GenBank Accession No. AW225337), 11 (or GenBank Accession No. AW225337), or 13 (or GenBank Accession No. AW225340}, can comfortably provide positive individual identification with a panel of perhaps 10 to 1,000 primers which each yield a noncoding amplified sequence of 100 bases. If predicted coding sequences of any of SEQ ID NO: 1, 3 (or GenBank Accession No. AW225336), 5 (or GenBank Accession No. AW225339), 7 (or GenBank Accession No. AW225341), 9 (or GenBank Accession No. AW225337), 11 (or GenBank Accession No. AW225337), or 13 (or GenBank Accession No. AW225340}, are used, a more appropriate number of primers for positive individual identification would be 500-2,000.

If a panel of reagents from the polynucleotides described herein is used to generate a unique identification database for an individual, those same reagents can later be used to identify tissue from that individual. Using the unique identification database, positive identification of the individual, living or dead, can be made from extremely small tissue samples.

5.6.6. Uses for Forensic Biology.

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample. This can be very useful in cases where a forensic pathologist is presented with a tissue of u unknown origin. Panels of such probes can be used to identify tissue by species and/or by organ type.

The polynucleotides of the invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions are particularly appropriate for this use as greater numbers of polymorphisms occur in the noncoding regions, making it easier to differentiate individuals using this technique. Examples of polynucleotide reagents include the polynucleotides of the invention or portions thereof, e.g., fragments derived from noncoding regions having a length of at least 20 or 30 bases.

Accordingly, the polynucleotides of the invention can be used to provide polynucleotide reagents, e.g., labeled probes that can be used in, for example, to identify a specific cell type or tissue type by in situ hybridization technique.

5.6.7. Predictive Medicine.

The present invention also pertains to the field of predictive medicine in which diagnostic assays, prognostic assays, pharmacogenomics, and monitoring clinical trails are used for prognostic (predictive) purposes to thereby treat an individual prophylactically. One aspect of the present invention relates to diagnostic assays for determining expression of a polypeptide or nucleic acid of the invention and/or activity of a polypeptide of the invention, in the context of a biological sample (e.g., blood, serum, cells, tissue) to thereby determine whether an individual is afflicted with a disease or disorder, or is at risk of developing a disorder, associated with aberrant expression or activity of a polypeptide of the invention, such as a proliferative disorder, e.g., cancer. Accordingly, the present invention provides a method for diagnosing a BCA-related disorder, comprising comparing an amount of BCA nucleic acid or BCA polypeptide expressed in a normal tissue to an amount expressed in a diseased tissue.

The invention also provides for prognostic (or predictive) assays for determining whether an individual is at risk of developing a disorder associated with aberrant expression or activity of a polypeptide of the invention. For example, mutations in a gene of the invention can be assayed in a biological sample. Such assays can be used for prognostic or predictive purpose to thereby prophylactically treat an individual prior to the onset of a disorder characterized by or associated with aberrant expression or activity of a polypeptide of the invention. Accordingly, the present invention provides a method for determining a prognosis of a BCA-related disorder, comprising the step of comparing an amount of BCA nucleic acid or BCA polypeptide expressed in a biological sample at a first stage of a disease to an amount of BCA nucleic acid or BCA polypeptide expressed in the sample at a second stage of the disease.

Another aspect of the invention provides methods for expression of a BCA nucleic acid or BCA polypeptide of the invention or activity of a BCA polypeptide of the invention in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent).

Yet another aspect of the invention pertains to monitoring the influence of agents (e.g., drugs or other compounds) on the in vivo expression or activity of a BCA polypeptide of the invention. These and other agents are described in further detail in the following sections.

5.6.8. Diagnostic Assays.

The present invention provides a method for diagnosing a BCA-related disorder, comprising comparing an amount of BCA nucleic acid or BCA polypeptide expressed in a normal tissue to an amount expressed in a diseased tissue. An exemplary method for detecting the presence or absence of a BCA polypeptide or BCA polynucleotide of the invention in a biological sample involves obtaining a biological sample from a test subject and contacting the biological sample with a compound or an agent capable of detecting a BCA polypeptide or nucleic acid (e.g., mRNA, genomic DNA) of the invention such that the presence of a polypeptide or nucleic acid of the invention is detected in the biological sample.

In a specific embodiment, the invention provides a method for diagnosing a BCA-related disorder in a subject comprising the steps of contacting a BCA antibody with a sample, suspected of containing a BCA polypeptide, from the subject under conditions that allow the BCA polypeptide to be bound by the BCA antibody and detecting or measuring binding of the BCA antibody to the BCA polypeptide, wherein detection or measurement of binding indicates presence or amount, respectively, of the BCA polypeptide, and wherein the BCA-related disorder is determined to be present when the presence or amount of detected BCA polypeptide differs from a control value representing the amount of BCA polypeptide present in an analogous sample from a subject not having the BCA-related disorder.

A preferred agent for detecting mRNA or genomic DNA encoding a polypeptide of the invention is a labeled nucleic acid probe capable of hybridizing to mRNA or genomic DNA encoding a polypeptide of the invention. The nucleic acid probe can be, for example, a full-length cDNA, such as the nucleic acid of SEQ ID NO: 1, 3 (or GenBank Accession No. AW225336), 5 (or GenBank Accession No. AW225339), 7 (or GenBank Accession No. AW225341), 9 (or GenBank Accession No. AW225337), 11 (or GenBank Accession No. AW225337), or 13 (or GenBank Accession No. AW225340}, or a portion thereof, such as an oligonucleotide of at least 15, 30, 50, 100, 250 or 500 contiguous nucleotides in length and sufficient to specifically hybridize under stringent conditions to a mRNA or genomic DNA encoding a polypeptide of the invention. Other suitable probes for use in the diagnostic assays of the invention are described herein.

A preferred agent for detecting a BCA polypeptide of the invention is an antibody capable of binding to a polypeptide of the invention, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

The term “biological sample” is intended to include tissues, cells and biological fluids isolated from a subject, as well as tissues, cells and fluids present within a subject. That is, the detection method of the invention can be used to detect mRNA, protein, or genomic DNA in a biological sample in vitro as well as in vivo. For example, in vitro techniques for detection of mRNA include Northern hybridizations and in situ hybridizations. In vitro techniques for detection of a polypeptide of the invention include enzyme linked immunosorbent assay (ELISA), Western blotting, immunoprecipitation and immunofluorescence. In vitro techniques for detection of genomic DNA include Southern hybridizations. Furthermore, in vivo techniques for detection of a polypeptide of the invention include introducing into a subject a labeled antibody directed against the polypeptide. For example, the antibody can be labeled with a radioactive marker whose presence and location in a subject can be detected by standard imaging techniques.

In one embodiment, the biological sample comprises protein molecules from the test subject. Alternatively, the biological sample can comprise mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting a BCA polypeptide of the invention or mRNA or genomic DNA encoding a polypeptide of the invention, such that the presence of the polypeptide or mRNA c, genomic DNA encoding the polypeptide is detected in the biological sample, and comparing the presence of the polypeptide or mRNA or genomic DNA encoding the polypeptide in the control sample with the presence of the polypeptide or mRNA or genomic DNA encoding the polypeptide in the test sample.

Probes based on the sequence of a BCA polynucleotide of the invention can be used to detect transcripts or genomic sequences encoding the same protein molecule encoded by a selected polynucleotide. The probe comprises a label group attached thereto, e.g., a radioisotope, a fluorescent compound, an enzyme, or an enzyme co-factor. Such probes can be used as part of a diagnostic test kit for identifying cells or tissues which mis-express the protein, such as by measuring levels of a polynucleotide encoding the protein in a sample of cells from a subject, e.g., detecting mRNA levels or determining whether a gene encoding the protein has been mutated or deleted.

5.6.8.1. Detection of BCA Gene Products

Antibodies directed against wild type or mutant BCA polynucleotides or polypeptides, or conserved variants or peptide fragments thereof, may also be used as diagnostics and prognostics, as described herein. Such diagnostic methods, may be used to detect abnormalities in the level of BCA gene expression, or abnormalities in the structure and/or temporal, tissue, cellular, or subcellular location of BCA gene product. Antibodies, or fragments of antibodies, such as those described below, may be used to screen potentially therapeutic compounds in vitro to determine their effects on BCA gene expression and BCA peptide production. The compounds which have beneficial effects on breast cancer can be identified and a therapeutically effective dose determined.

The tissue or cell type to be analyzed will generally include those which are known, or suspected, to express the BCA gene, such as, for example, breast cancer cells or metastatic cells. The protein isolation methods employed herein may, for example, be such as those described in Harlow and Lane (Harlow, E. and Lane, D., 1988, “Antibodies: A Laboratory Manual”, Cold Spring Harbor Laboratory Press, Cold Spring Harbor, N.Y.), which is incorporated herein by reference in its entirety. The isolated cells can be derived from cell culture or from a patient. The analysis of cell taken from culture may be a necessary step to test the effect of compounds on the expression of the BCA gene.

Preferred diagnostic methods for the detection of BCA gene products or conserved variants or peptide fragments thereof, may involve, for example, immunoassays wherein the BCA gene products or conserved variants, including gene products which are the result of alternatively spliced transcripts, or peptide fragments are detected by their interaction with an anti-BCA gene product-specific antibody.

For example, antibodies, or fragments of antibodies, useful in the present invention may be used to quantitatively or qualitatively detect the presence of BCA gene products or conserved variants or peptide fragments thereof. The antibodies (or fragments thereof) useful in the present invention may, additionally, be employed histologically, as in immunofluorescence or immunoelectron microscopy, for in situ detection of BCA gene products or conserved variants or peptide fragments thereof. In situ detection may be accomplished by removing a histological specimen from a patient, such as paraffin embedded sections of breast tissues and applying thereto a labeled antibody of the present invention. The antibody (or fragment) is preferably applied by overlaying the labeled antibody (or fragment) onto a biological sample. When a BCA gene product is present in the cytoplasm, the antibody of the invention can be introduced inside the cell, for example, by making the cell membrane permeable. Through the use of such a procedure, it is possible to determine not only the presence of a BCA gene product, or conserved variants or peptide fragments, but also the distribution of a BCA in a cell, tissue, or organ of interest. Using the present invention, those of ordinary skill will readily perceive that any of a wide variety of histological methods (such as staining procedures) can be modified in order to achieve such in situ detection.

Immunoassays for BCA gene products or conserved variants or peptide fragments thereof will typically comprise incubating a sample, such as a biological fluid, a tissue extract, freshly harvested cells, or lysates of cells which have been incubated in cell culture, in the presence of a detectably labeled antibody capable of identifying BCA gene products or conserved variants or peptide fragments thereof, and detecting the bound antibody by any of a number of techniques well-known in the art.

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labeled BCA gene specific antibody. The solid phase support may then be washed with the buffer a second time to remove unbound antibody. The amount of bound label on solid support may then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given lot of anti-BCA gene product antibody may be determined according to standard methods. Those skilled in the art will be able to determine operative and optimal assay conditions for each determination by using standard techniques.

One of the ways in which the BCA gene peptide-specific antibody can be detectably labeled is by linking the same to an enzyme and use in an enzyme immunoassay (EIA) (Voller, 1978,“The Enzyme Linked Immunosorbent Assay (ELISA)”, Diagnostic Horizons 2:1-7, Microbiological Associates Quarterly Publication, Walkersville, Md.); Voller et al., 1978, J. Clin. Pathol. 31:507-520; Butler, 1981, Meth. Enzymol. 73:482-523; Maggio, E. (ed.), 1980, Enzyme Immunoassay, CRC Press, Boca Raton, Fla.; Ishikawa, E. et al., (eds.), 1981, Enzyme Immunoassay, Kgaku Shoin, Tokyo). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect BCA peptides through the use of a radioimmunoassay (RIA) (See, e.g., Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a gamma counter or a scintillation counter or by autoradiography.

It is also possible to label the antibody with a fluorescent compound. When the fluorescently labeled antibody is exposed to light of the proper wavelength, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

In various embodiments, the present invention provides the measurement of BCA gene products, and the uses of such measurements in clinical applications.

As used herein, the term “soluble” shall mean those molecules that are released by normal or pathologic physiological processes of a cell.

The measurement of BCA gene product of the invention can be valuable in detecting and/or staging breast cancer in a subject, in screening of breast cancer in a population, in differential diagnosis of the physiological condition of a subject, and in monitoring the effect of a therapeutic treatment on a subject.

The present invention also provides for the detecting, diagnosing, or staging of breast cancer, or the monitoring of treatment of breast cancer by measuring in addition to one or more BCA1-7 gene products at least one other marker, such as receptors or differentiation antigens. For example, serum markers selected from, for example but not limited to, carcinoembryonic antigen (CEA), CA15-3, CA549, CAM26, M29, CA27.29 and MCA can be measured in combination with one or more BCA1-7 gene products to detect, diagnose, stage, or monitor treatment of breast cancer. In another embodiment, the prognostic indicator is the observed change in different marker levels relative to one another, rather than the absolute levels of the markers present at any one time. These measurements can also aid in predicting therapeutic outcome and in evaluating and monitoring the overall disease status of a subject.

In a specific embodiment of the invention, a soluble BCA gene product alone or in combination with other markers can be measured in any body fluid of the subject including but not limited to blood, serum, plasma, milk, urine, saliva, pleural effusions, synovial fluid, spinal fluid, tissue infiltrations and tumor infiltrates. The measurements of soluble BCA gene products in blood or serum are preferred with respect to the development of a test kit which is to be used in clinics and homes.

Any of numerous immunoassays can be used in the practice of the instant invention. Antibodies, or antibody fragments comprising the binding domain, are known in the art or can be obtained by procedures standard in the art such as those described in Harlow and Lane, Antibodies: A Laboratory Manual, Cold Spring Harbor Laboratory, 1988.

5.6.8.2. Detecting and Staging Breast Cancer in a Patient

Cancer can be detected and staged in a patient using a BCA polynucleotide and polypeptide of the invention. In one embodiment of the present invention, measurement of at least one BCA1-7 gene products or fragments thereof, or soluble BCA1-7 gene products can be used to detect breast cancer in a subject or to stage the breast cancer in a subject.

Staging refers to the grouping of patients according to the extent of their disease. Staging is useful in choosing treatment for individual patients, estimating prognosis, and comparing the results of different treatment programs. Staging of breast cancer is performed initially on a clinical basis, according to the physical examination and laboratory radiologic evaluation. The most widely used clinical staging system is the one adopted by the International Union against Cancer (UICC) and the American Joint Committee on Cancer (AJCC) Staging and End Results Reporting. It is based on the tumor-nodes-metastases (TNM) system as detailed in the 1988 Manual for Staging of Cancer.

Accordingly, in an exemplary embodiment, the invention provides a method for staging a BCA-related disorder in a subject comprising the steps of contacting a BCA antibody with a sample, suspected of containing a BCA polypeptide, from the subject under conditions that allow the BCA polypeptide to be bound by the BCA antibody and detecting or measuring binding of the BCA antibody to the BCA polypeptide, wherein detection or measurement of binding indicates presence or amount, respectively, of the BCA polypeptide, and wherein the stage of a BCA-related disorder in a subject is determined to be present when the presence or amount of detected BCA polypeptide is compared with the amount of BCA polypeptide present in an analogous sample from a subject having a particular stage of a BCA-related disorder.

5.6.9. Prognostic Assays.

The methods described herein can furthermore be utilized as prognostic assays to identify subjects having or at risk of developing a disease or disorder associated with aberrant expression or activity of a BCA polypeptide of the invention. For example, the assays described herein, such as the preceding diagnostic assays or the following assays, can be utilized to identify a subject having or at risk of developing a disorder associated with aberrant expression or activity of a BCA polypeptide of the invention, e.g., an immunologic disorder, or embryonic disorders. Alternatively, the prognostic assays can be utilized to identify a subject having or at risk for developing such a disease or disorder. Thus, the present invention provides a method in which a test sample is obtained from a subject and a BCA polypeptide or nucleic acid (e.g., mRNA, genomic DNA) of the invention is detected, wherein the presence of the polypeptide or nucleic acid is diagnostic for a subject having or at risk of developing a disease or disorder associated with aberrant expression or activity of the polypeptide. As used herein, a “test sample” refers to a biological sample obtained from a subject of interest. For example, a test sample can be a biological fluid (e.g., serum), cell sample, or tissue.

The prognostic assays described herein, for example, can be used to identify a subject having or at risk of developing disorders such as cancers, for example, hormone-sensitive cancer such as breast cancer.

In another example, prognostic assays described herein can be used to identify a subject having or at risk of developing related disorders associated with expression of polypeptides of the invention.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant expression or activity of a polypeptide of the invention. For example, such methods can be used to determine whether a subject can be effectively treated with a specific agent or class of agents (e.g., agents of a type which decrease activity of the polypeptide). Thus, the present invention provides methods for determining whether a subject can be effectively treated with an agent for a disorder associated with aberrant expression or activity of a polypeptide of the invention in which a test sample is obtained and the polypeptide or nucleic acid encoding the polypeptide is detected (e.g., wherein the presence of the polypeptide or nucleic acid is diagnostic for a subject that can be administered the agent to treat a disorder associated with aberrant expression or activity of the polypeptide).

The methods of the invention can also be used to detect genetic lesions or mutations in a BCA gene of the invention, thereby determining if a subject with the lesioned gene is at risk for a disorder characterized by aberrant expression or activity of a polypeptide of the invention. In preferred embodiments, the methods include detecting, in a sample of cells from the subject, the presence or absence of a genetic lesion or mutation characterized by at least one of an alteration affecting the integrity of a gene encoding the polypeptide of the invention, or the mis-expression of the gene encoding the polypeptide of the invention. For example, such genetic lesions or mutations can be detected by ascertaining the existence of at least one of: 1) a deletion of one or more nucleotides from a BCA gene; 2) an addition of one or more nucleotides to a BCA gene; 3) a substitution of one or more nucleotides of a BCA gene; 4) a chromosomal rearrangement of a BCA. gene; 5) an alteration in the level of a messenger RNA transcript of a BCA gene; 6) an aberrant modification of a BCA gene, such as of the methylation pattern of the genomic DNA; 7) the presence of a non-wild type splicing pattern of a messenger RNA transcript of a BCA gene; 8) a non-wild type level of the protein encoded by a BCA gene; 9) an allelic loss of a BCA gene; and 10) an inappropriate post-translational modification of the protein encoded by a BCA gene. As described herein, there are a large number of assay techniques known in the art which can be used for detecting lesions in a gene.

In certain embodiments, detection of the lesion involves the use of a probe/primer in a polymerase chain reaction (PCR) (See, e.g., U.S. Pat. Nos. 4,683,195 and 4,683,202), such as anchor PCR or RACE PCR, or, alternatively, in a ligation chain reaction (LCR) (see, e.g., Landegran et al., 1988, Science 241:1077-1080; Nakazawa et al., 1994, Proc Natl Acad Sci. 91:360-364), the latter of which can be particularly useful for detecting point mutations in a gene (See, e.g., Abravaya et al., 1995, Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to the selected gene under conditions such that hybridization and amplification of the gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli et al., 1990, Proc Natl Acad Sci. 87:1874-1878), transcriptional amplification system (Kwoh, et al., 1989, Proc Natl Acad Sci. 86:1173-1177), Q-Beta Replicase (Lizardi et al., 1988, BioTechnology 6:1197), or any other nucleic acid amplification method, followed by the detection of the amplified molecules using techniques well known to those of skill in the art. These detection schemes are especially useful for the detection of polynucleotides if such molecules are present in very low numbers.

In an alternative embodiment, mutations in a selected gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,498,531) can be used to score for the presence of specific mutations by development or loss of a ribozyme cleavage site.

In other embodiments, genetic mutations can be identified by hybridizing a sample and control nucleic acids, e.g., DNA or RNA, to high density arrays comprising hundreds or thousands of oligonucleotides probes (Cronin et al., 1996, Human Mutation 7:244-255; Kozal et al., 1996. Nature Medicine 2:753-759). For example, genetic mutations can be identified in two-dimensional arrays containing light-generated DNA probes as described in Cronin et al., supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the selected gene and detect mutations by comparing the sequence of the sample nucleic acids with the corresponding wild-type (control) sequence. Examples of sequencing reactions include those based on techniques developed by Maxim and Gilbert (1977, Proc Natl Acad Sci. 74:560) or Sanger (1977, Proc Natl Acad Sci. 74:5463). It is also contemplated that any of a variety of automated sequencing procedures can be utilized when performing the diagnostic assays, including sequencing by mass spectrometry (see, e.g., PCT Publication No. WO 94/16101; Cohen et al., 1996 Adv Chromatogr. 36:127-162; Naeve et al., 1995, “Accuracy of automated DNA sequencing: a multi-laboratory comparison of sequencing results”, Biotechniques. 19:448-453; Griffin et al., 1993, Appl Biochem Biotechnol. 38:147-159).

Other methods for detecting mutations in a selected gene include methods in which protection from cleavage agents is used to detect mismatched bases in RNA/RNA or RNA/DNA heteroduplexes (Myers et al., 1985, Science 230:1242). In general, the technique of “mismatch cleavage” entails providing heteroduplexes formed by hybridizing (labeled) RNA or DNA comprising the wild-type sequence with potentially mutant RNA or DNA obtained from a tissue sample. The double-stranded duplexes are treated with an agent which cleaves single-stranded regions of the duplex such as which will exist due to basepair mismatches between the control and sample strands. RNA/DNA duplexes can be treated with RNase to digest mismatched regions, and DNA/FDNA hybrids can be treated with S1 nuclease to digest mismatched regions.

In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton et al., 1988, Proc Natl Acad Sci. 85:4397; Saleeba et al., 1992, Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in cDNAs obtained from samples of cells. For example, the mutY enzyme of E. coli cleaves A at G/A mismatches and the thymidine DNA glycosylase from HeLa cells cleaves T at G/T mismatches (Hsu et al., 1994, Carcinogenesis 15:1657-1662). According to an exemplary embodiment, a probe based on a selected sequence, e.g., a wild-type sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in genes. For example, single strand conformation polymorphism (SSCP) may be used to detect differences in electrophoretic mobility between mutant and wild type nucleic acids (see, e.g., Orita et al., 1989, Proc Natl Acad Sci. 86:2766; Cotton, 1993, Mutat Res. 285:125-144; Hayashi, 1992, Genet Anal Tech Appl. 9:73-79). Single-stranded DNA fragments of sample and control nucleic acids will be denatured and allowed to renature. The secondary structure of single-stranded nucleic acids varies according to sequence, and the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a prefered embodiment, the subject method utilizes heteroduplex analysis to separate double stranded heteroduplex molecules on the basis of changes in electrophoretic mobility (Keen et al., 1991, Trends Genet. 7:5).

In yet another embodiment, the movement of mutant or wild-type fragments in polyacrylamide gels containing a gradient of denaturant is assayed using denaturing gradient gel electrophoresis (DGGE) (Myers et al., 1985, Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner, 1987, Biophys Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al., 1986, Nature 324:163); Saiki et al., 1989, Proc Natl Acad Sci. 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al., 1989, Nucleic Acids Res. 17:2437-2448) or at the extreme 3′ end of one primer where, under appropriate conditions, mismatch can prevent or reduce polymerase extension (Prossner, 1993, Tibtech 11:238). In addition, it may be desirable to introduce a novel restriction site in the region of the mutation to create cleavage-based detection (Gasparini et al., 1992, Mol. Cell Probes 6:1). It is anticipated that in certain embodiments amplification may also be performed using Taq ligase for amplification (Barany, 1991, Proc Natl Acad Sci. 88:189). In such cases, ligation will occur only if there is a perfect match at the 3′ end of the 5′ sequence making it possible to detect the presence of a known mutation at a specific site by looking for the presence or absence of amplification.

Furthermore, the presence of a BCA polynucleotide or polypeptide of the invention can be correlated with the presence or expression level of other cancer-related proteins, such as for example, androgen receptor, estrogen receptor, adhesion molecules (e.g., E-cadherin), proliferation markers (e.g., MIB-1), tumor-suppressor genes (e.g., syk, TP53, retinoblastoma gene product), vascular endothelial growth factor (Lissoni et al., 2000, “Chemotherapy and angiogenesis in advanced cancer: vascular endothelial growth factor (VEGF) decline as predictor of disease control during taxol therapy in metastatic breast cancer”, Int J Biol Markers. 15:308-11), Rad51 (Maacke et al., 2000, “Over-expression of wild-type Rad51 correlates with histological grading of invasive ductal breast cancer”, Int J Cancer. 88:907-913), cyclin D1, BRCA1, BRCA2, or carcinoembryonic antigen.

The methods described herein may be performed, for example, by utilizing pre-packaged diagnostic kits comprising at least one nucleic acid probe or antibody reagent described herein, which may be conveniently used, e.g., in clinical settings to diagnose patients exhibiting symptoms or family history of a disease or illness involving a gene encoding a polypeptide of the invention. Further more, any cell type or tissue, e.g., preferably peripheral blood leukocytes, in which the polypeptide of the invention is expressed may be utilized in the prognostic assays described herein.

5.6.10. Pharmacogenomics.

Pharmacogenomics deals with clinically significant hereditary variations in the response to drugs due to altered drug disposition and abnormal action in affected persons (see, e.g., Linder, 1997, Clin Chem. 43:254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism”. These pharmacogenetic conditions can occur either as rare defects or as polymorphisms.

Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of a BCA polypeptide of the invention, expression of a BCA nucleic acid of the invention, or mutation content of a BCA gene of the invention in an individual can be determined to thereby select appropriate agent(s) for therapeutic or prophylactic treatment of the individual.

Accordingly, in addition to the nucleotide sequences of SEQ ID NO: 1, 3 (or GenBank Accession No. AW225336), 5 (or GenBank Accession No. AW225339), 7 (or GenBank Accession No. AW225341), 9 (or GenBank Accession No. AW225337), 11 (or GenBank Accession No. AW225338), and 13 (or GenBank Accession No. AW225340), it will be appreciated by those skilled in the art that DNA sequence polymorphisms that lead to changes in the amino acid sequence may exist within a population (e.g., the human population). Such genetic polymorphisms may exist among individuals within a population due to natural allelic variation. An allele is one of a group of genes which occur alternatively at a given genetic locus. As used herein, the phrase “allelic variant” refers to a nucleotide sequence which occurs at a given locus or to a polypeptide encoded by the nucleotide sequence. Such natural allelic variations can typically result in 1-5% variance in the nucleotide sequence of a given gene. Alternative alleles can be identified by sequencing the gene of interest in a number of different individuals or by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

5.6.11. Prophylactic Methods.

The present invention provides for prophylactic and therapeutic methods of treating a subject at risk of or having a BCA-related disorder. Such a BCA-related disorder includes, but is not limited to, an allergy, anxiety disorder, autoimmune disease, behavioral disorder, birth defect, blood disorder, bone disease, cancer, circulatory disease, tooth disease, depressive disorder, dissociative disorder, ear condition, eating disorder, eye condition, food allergy, food-borne illness, gastrointestinal disease, genetic disorder, heart disease, hormonal disorder, infectious disease, insect-transmitted disease, nutritional disorder, kidney disease, leukodystrophy, liver disease, mental health disorder, metabolic disease, mood disorder, neurological disorder, neurodegenerative disorder, personality disorder, phobia, pregnancy complication, prion disease, prostate disease, respiratory disease, sexual disorder, skin condition, sleep disorder, speech-language disorder, sports injury, tropical disease, vestibular disorder, prostate cancer, acquired immunodeficiency syndrome, hepatitis or breast cancer.

Accordingly, the present invention provides for prophylactic and therapeutic methods of treating a subject at risk of (or susceptible to) a disorder or having a disorder associated with aberrant expression or activity of a BCA polypeptide of the invention. For example, disorders characterized by aberrant expression or activity of the polypeptides of the invention include hormone-sensitive cancers, such as but not limited to cancer of the breast, ovary, uterus, prostate, testis, skin and brain.

In one aspect, the invention provides a method for preventing in a subject, a disease or condition associated with an aberrant expression or activity of a BCA polypeptide of the invention, by administering to the subject an agent which modulates expression of at least one activity of a BCA polypeptide. Subjects at risk for a disease which is caused or contributed to by aberrant expression or activity of a BCA polypeptide of the invention can be identified by, for example, any or a combination of diagnostic or prognostic assays as described herein. Administration of a prophylactic agent can occur prior to the manifestation of symptoms characteristic of the aberrancy, such that a disease or disorder is prevented or, alternatively, delayed in its progression. Depending on the type of aberrancy, for example, an agonist or antagonist agent can be used for treating the subject.

The prophylactic agents described herein, for example, can be used to treat a subject at risk of developing disorders such as disorders discussed for example, in Sections above relative to the uses of the sequences of the invention. For example, an antagonist of a BCA polypeptide of the invention can be used to modulate or treat breast cancer. The appropriate agent can be determined based on screening assays described herein.

5.6.12. Methods of Treatment.

The present invention encompasses methods for the prevention and/or treatment of a BCA-related disorder comprising administering to a patient in need thereof a BCA polynucleotide, BCA polypeptide, BCA agonist, BCA antagonist, or an inhibitor of a BCA agonist or antagonist.

More particularly, the present invention relates to uses of BCA polynucleotides, polypeptides, and BCA antagonists for the prevention, diagnosis, prognosis and management of cancer, preferably hormone-sensitive cancers, such as but not limited to cancer of the breast, ovary, uterus, prostate, testis, skin and brain. The present invention is based, at least in part, on the discovery of cDNA molecules which encode BCA proteins that are more highly expressed in breast cancer cells than in normal breast cells.

Cancer describes a disease state in which a carcinogenic agent or agents causes the transformation of a healthy cell into an abnormal cell, which is followed by an invasion of adjacent tissues by these abnormal cells, and which may be followed by lymphatic or blood-borne spread of these abnormal cells to regional lymph nodes and/or distant sites, i.e., metastasis.

Abnormal cell regulation may lead to tumor growth such that the tissue mass is increased because of greater cell numbers as a result of faster cell division and/or slower rates of cell death. Tumors may be malignant or non-malignant.

The invention contemplates uses of BCA nucleic acids, polypeptides, and BCA antagonists (e.g., antibodies directed against BCA polypeptides of the invention) to treat cancer, i.e., to inhibit the replication of cancer cells, inhibit the spread of cancer, decrease tumor size, lessen or reduce the number of cancerous cells in the body, or ameliorate or alleviate the symptoms of the disease caused by the cancer. Such treatment is considered therapeutic if there is a decrease in mortality and/or morbidity, or a decrease in disease burden manifest by reduced numbers of malignant cells in the body.

The invention contemplates uses of BCA nucleic acids, polypeptides, and BCA antagonists (e.g., antibodies directed against BCA polypeptides of the invention) to prevent cancer, i.e., to prevent the occurrence or recurrence of the disease state of cancer. As such, a treatment that impedes, inhibits, or interferes with metastasis, tumor growth, or cancer proliferation has preventive activity.

In one embodiment, the present invention provides a method for identifying an inhibitor of growth of a breast cancer cell comprising the steps of contacting the cell with a BCA polynucleotide, BCA polypeptide, or an BCA antagonist (e.g., an antibody directed against a BCA polypeptide and that immunospecifically binds to a BCA polypeptide), and measuring cell growth, wherein the polynucleotide or polypeptide is selected from the group consisting of a BCA1, BCA2, BCA3, BCA4, BCA5, BCA6, and BCA7 polynucleotide or polypeptide; and wherein an inhibition of cell growth indicates the presence of an inhibitor of growth of a breast cancer cell.

In a specific embodiment, the present invention provides a method for inhibiting the degradation of a tumor suppressor comprising administering to a patient in need thereof an effective amount of a BCA nucleic acid, BCA polypeptide, BCA agonist, BCA antagonist or a variant thereof. Many tumor suppressors are known in the art (see, e.g., Hakem and Mak, 2001, “Animal models of tumor-suppressor genes”, Annu Rev Genet. 35:209-241). Specific target molecules include, but are not limited to, a protein that increases ubiquitination of a tumor suppressor, and therefore promotes degradation of the tumor suppressor. BCA polypeptides that bind such a protein and inhibit its activity can increase the half-life of the tumor suppressor, and can reduce the risk of, or treat, tumor formation or metastasis.

Similarly, by targeting molecules that bind and inhibit or inactivate a tumor suppressor, the half-life of the tumor suppressor can be increased, and thereby reduce the risk of, or treat, tumor formation or metastasis.

In another specific embodiment the present invention provides a method for promoting or facilitating the degradation of an oncogenic protein (e.g., a polypeptide encoded by an oncogene) comprising administering to a patient in need thereof an effective amount of a BCA nucleic acid, BCA polypeptide, BCA agonist, BCA antagonist or a variant thereof.

In another specific embodiment, the present invention provides a method for inhibiting the activity of an oncogenic protein (e.g., a polypeptide encoded by an oncogene) comprising administering to a patient in need thereof an effective amount of a BCA nucleic acid, BCA polypeptide, BCA agonist, BCA antagonist or a variant thereof. For example, BCA polypeptides that bind such a oncogenic protein and inhibit its activity can reduce the risk of, or treat, tumor formation or metastasis.

As such, specific target molecules include, but are not limited to, any oncogene known in the art (see, e.g., Chin and DePinho, 2000, “Flipping the oncogene switch: illumination of tumor maintenance and regression”, Trends Genet. 16(4):147-150).

Accordingly, the invention pertains to methods of modulating BCA expression or activity of a BCA polypeptide of the invention for therapeutic purposes. The modulatory method of the invention involves contacting a cell with an agent that modulates one or more of the activities of the polypeptide. An agent that modulates activity can be an agent as described herein, such as a nucleic acid or a protein, a naturally-occurring cognate ligand of the polypeptide, a peptide, a peptidomimetic, or other small molecules. In one embodiment, the agent stimulates one or more of the biological activities of the polypeptide. Examples of such stimulatory agents include the active polypeptide of the invention and a polynucleotide encoding the polypeptide of the invention that has been introduced into the cell.

In another embodiment, the agent inhibits one or more of the biological activities of the polypeptide of the invention. Examples of such inhibitory agents include antisense polynucleotides and antibodies. These modulatory methods can be performed in vitro (e.g., by culturing the cell with the agent) or, alternatively, in vivo (e.g., by administering the agent to a subject or in the vicinity of the cells). As such, the present invention provides methods of treating an individual afflicted with a disease or disorder characterized by aberrant expression or activity of a BCA polypeptide of the invention. In one embodiment, the method involves administering an agent (e.g., an agent identified by a screening assay described herein), or combination of agents that modulates (e.g., upregulates or downregulates) expression or activity. In another embodiment, the method involves administering a BCA polypeptide of the invention or a polynucleotide of the invention as therapy to compensate for reduced or aberrant expression or activity of the polypeptide.

Stimulation of activity is desirable in situations in which activity or expression is abnormally low or downregulated and/or in which increased activity is likely to have a beneficial effect. Conversely, inhibition of activity is desirable in situations in which activity or expression is abnormally high or upregulated and/or in which decreased activity is likely to have a beneficial effect.

5.6.13. Cancers and Therapeutics.

BCA nucleic acids, BCA polypeptides, and modulators thereof, can be used to modulate the development and progression of non-cancerous cell-proliferative disorders such as, but not limited to, deregulated proliferation (e.g., hyperdysplasia, hyper-IgM syndrome, or lymphoproliferative disorders), cirrhosis of the liver (a condition in which scarring has overtaken normal liver regeneration processes), treatment of keloid (hypertrophic scar) formation (disfiguring of the skin in which the scarring process interferes with normal renewal), psoriasis (a common skin condition characterized by excessive proliferation of the skin and delay in proper cell fate determination), benign tumors, fibrocystic conditions, and tissue hypertrophy (e.g., prostatic hyperplasia).

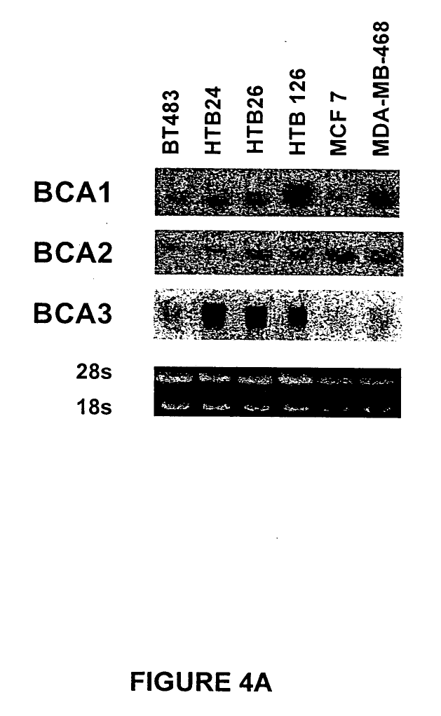

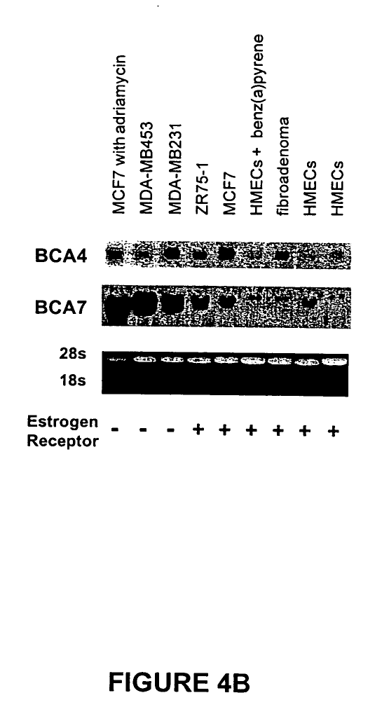

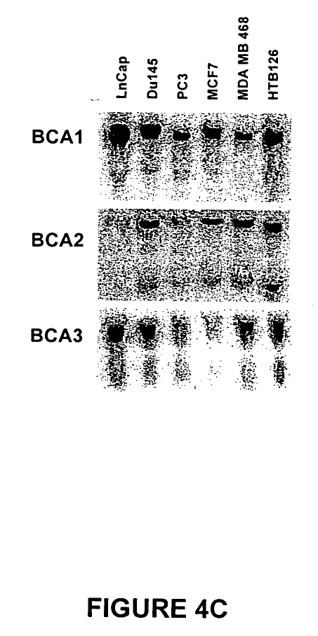

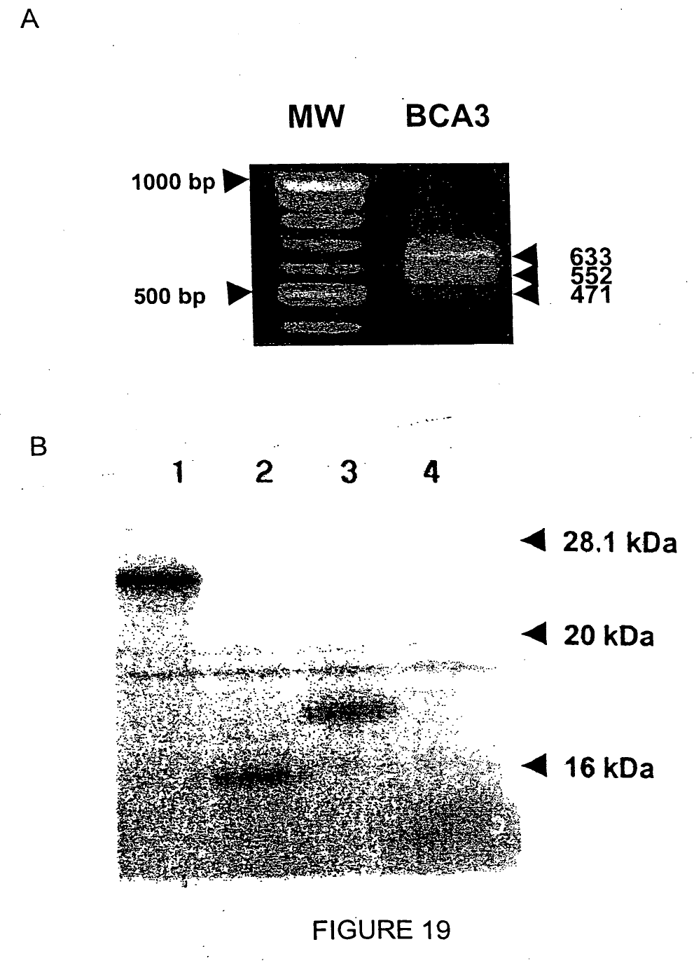

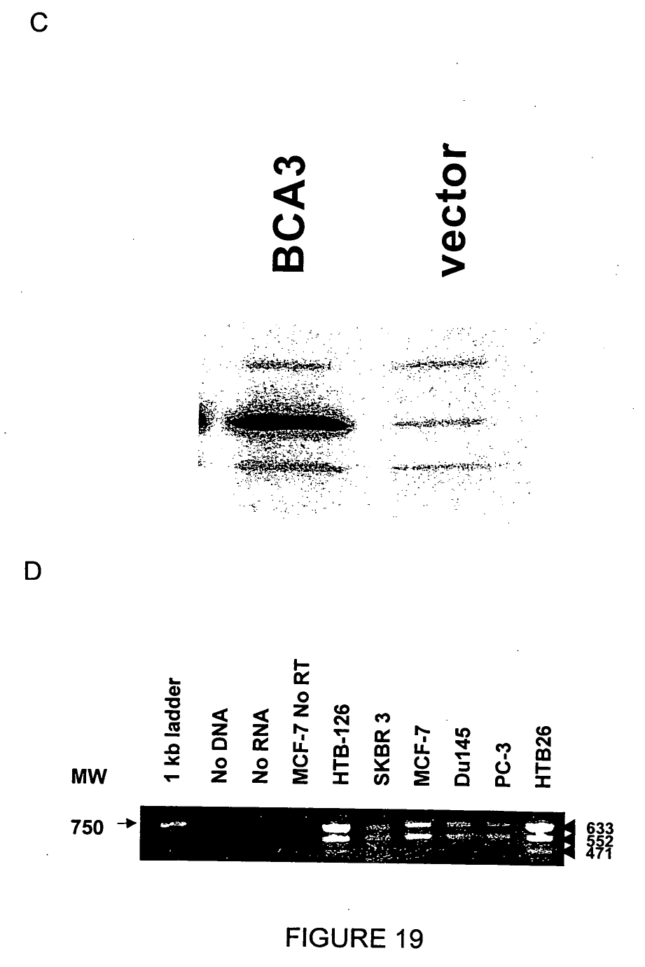

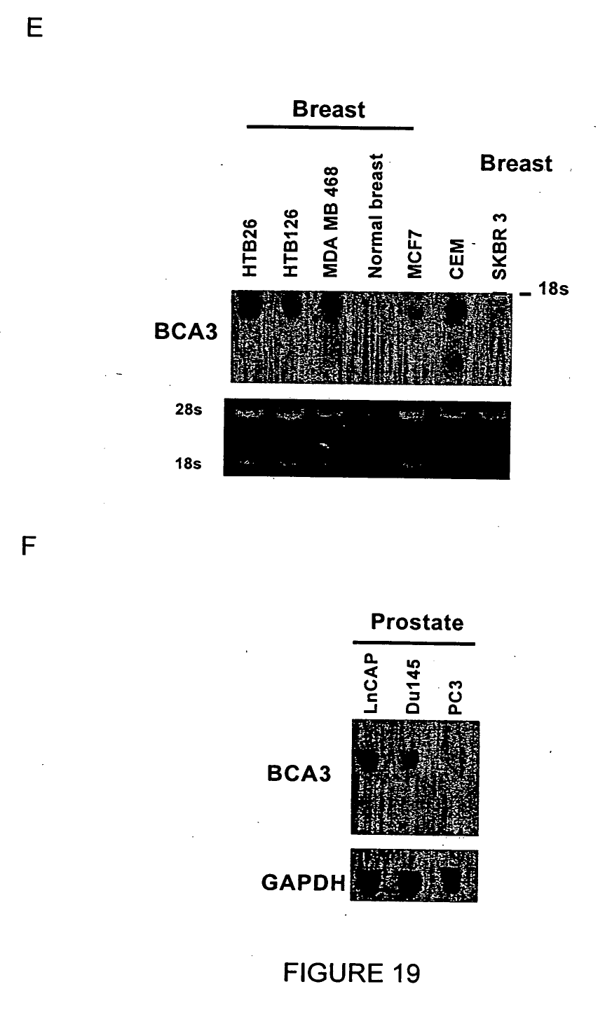

BCA nucleic acids, BCA polypeptides, and modulators thereof, can also be used to. modulate the development and progression of cancers such as, but not limited to, neoplasms, tamors, carcinomas, sarcomas, adenomas or myeloid lymphoma tumors, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leimyosarcoma, rhabdotheliosarcoma, colon sarcoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hematoma, bile duct carcinoma, melanoma, choriocarcinoma, semicoma, embryonal carcinoma, Wilms' tumor, cervical cancer, testicular tumor, lung carcinoma, small cell carcinoma, bladder carcinoma, epithelial carcinoma, glioma, astrocytoma, medulloblastoma, craniopharyngioma, ependynoma, pinealoma, hemangioblastoma, retinoblastoma), leukemias, (e.g. acute lymphocytic leukemia), acute myelocytic leukemia (myelolastic, promyelocytic, myelomonocytic, monocytic and erythroleukemia), chronic leukemias (chronic myelocytic (granulocytic) leukemia and chronic lymphocytic leukemia), or polycythemia vera, or lymphomas (Hodgkin's disease and non-Hodgkin's diseases), multiple myelomas and Waldenström's macroglobulinemia. In particular, the BCA polynucleotides, polypeptides, and modulators thereof, can be used to modulate the development and progression of hormone-sensitive cancers, such as but not limited to cancer of the breast, ovary, uterus, prostate, testis, skin and brain. Cancers and related cancer cell lines that showed an elevated expression level of BCA proteins are particularly suited, e.g, prostate cancer and BCA3, see FIGS. 4C and 19E.

5.6.14. Antisense Therapy.

The present invention provides compositions and methods for the use of a BCA antisense oligonucleotide to prevent or treat a BCA-related disorder, such as cancer, in particular breast cancer. The invention also provides pharmaceutical compositions comprising a BCA antisense oligonucleotide, as well as methods for their prophylactic and therapeutic use. An antisense oligonucleotide, or an analogue or derivative thereof, refers to a range of chemical species that recognize polynucleotide target sequences through Watson-and-Crick hydrogen bonding interactions with the nucleotide bases of the target sequences. The target sequences may be RNA or DNA, and may be single-stranded or double-stranded. Target molecules include, but are not limited to, pre-rmRNA, mRNA, and DNA. Also encompassed by the invention are drug delivery means and therapeutic regimens for the pharmaceutical compositions of the invention.

In one embodiment, a BCA antisense oligonucleotide is administered to a human to prevent or treat cancer, wherein BCA mRNA or protein is expressed at above-normal levels.

In another embodiment, a BCA antisense oligonucleotide is administered to a human at a high dose to prevent or treat cancer.

In another embodiment, a BCA antisense oligonucleotide is administered to a human at a low or reduced dose to prevent or treat cancer.

Aside from affecting diseased tissue, a BCA antisense oligonucleotide can affect normal tissues, which include tissues containing cells that normally express a BCA gene. Additionally, a BCA antisense oligonucleotide can affect normal tissues that, although not expressing a BCA gene, are compromised by diseased tissues. In a particular embodiment, a BCA antisense oligonucleotide can protect normal tissues that do or do not normally express a BCA gene.

In one embodiment, a BCA antisense oligonucleotide is administered to prevent or treat cancer, to a patient in need of such treatment, for a short treatment cycle.

In another embodiment, the invention further encompasses combination therapy to prevent or treat cancer. Such therapy includes the use of one or more molecules, compounds or treatments that assist in the prevention or treatment of a disease. Examples of contemplated therapeutics include biologicals, chemicals, and therapeutic treatments (e.g., irradiation treatment). Accordingly, the present invention provides for preventing or treating cancer comprising administering, to a patient in need of such treatment, a pharmaceutical composition, which comprises a BCA antisense oligonucleutide, and one or more therapeutic agents, such that the BCA antisense oligonucleotide potentiates the effect of additional therapeutic agents, and thereby reduces the overall toxicity of a therapeutic regimen. For example, lower dosages, fewer administrations, and shorter treatment periods can demonstrate fewer side effects or improved efficacy as compared to most standard treatments, such as those for cancer.

In a specific embodiment, the invention provides for a BCA antisense oligonucleotide that is administered to a human, in combination with one of more cancer therapeutic agents, to prevent or treat cancer. Such cancer therapeutics include one or more molecules, compounds or treatments that have anti-cancer activity. Examples of contemplated cancer therapeutics include biologicals, chemicals, and therapeutic treatments (e.g., irradiation treatment).

In another specific embodiment, the invention provides for a BCA antisense oligonucleotide that is administered to a human, in combination with one of more cancer therapeutic agents at reduced doses, to prevent or treat cancer. Such treatments may involve high, standard, or low doses of one or more BCA antisense oligonucleotides, and treatment cycles may be of long or short duration. In a specific embodiment, the invention provides for a particularly high dose of a BCA antisense oligonucleotide that is administered to a human, in combination with one of more cancer therapeutic agents at reduced doses, for short treatment cycles to prevent or treat cancer.

The invention described herein encompasses a method of preventing or treating cancer comprising a therapeutically effective amount of a BCA antisense oligonucleotide to a human in need of such therapy. The invention further encompasses the use of a short period of administration of a BCA antisense oligonucleotide. Examples of types of cancer, include, but are not limited to, non-Hodgkin's lymphoma, Hodgkin's lymphoma, leukemia (e.g., acute leukemia such as acute lymphocytic leukemia, acute myelocytic leukemia, chronic myeloid leukemia, chronic lymphocytic leukemia, multiple myeloma), colon carcinoma, rectal carcinoma, pancreatic cancer, breast cancer, ovarian cancer, prostate cancer, renal cell carcinoma, hepatoma, bile duct carcinoma, choriocarcinoma, cervical cancer, testicular cancer, lung carcinoma, bladder carcinoma, melanoma, head and neck cancer, brain cancer, cancers of unknown primary site, neoplasms, cancers of the peripheral nervous system, cancers of the central nervous system, tumors (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, seminoma, embryonal carcinoma, Wilms' tumor, small cell lung carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, and retinoblastoma), heavy chain disease, metastases, or any disease or disorder characterized by uncontrolled or abnormal cell growth.

5.6.15. Antibody Therapy.

The present invention contemplates uses of the BCA gene product specific antibodies of the invention for the prevention or treatment of cancer, particularly breast cancer. Examples of such antibody therapy are well known in the art, using antibodies such as Herceptin®, Rituxan®, OvaRex, Panorex, BEC2, IMC-C225, Vitaxin, Campath I/H, Smart MI95, LymphoCide, Smart I D10, and Oncolym.

Accordingly, an antibody directed to any of BCA1-7 can be used for antibody therapy, following routine procedures for the development of antibodies for use in clinical settings. Depending on the disease to be treated, approximately 1 μg/kg to 20 mg/kg of a pharmaceutical comprising an antibody of the present invention is administered to the patient. In one embodiment, the dose of antibody is 1 μg/kg to 100 μg/kg. In another embodiment, the dose of antibody is 101 μg/kg to 999 μg/kg. In another embodiment, the dose of antibody is 1 mg/kg to 5 mg/kg. In yet another embodiment, the dose of antibody is 6 mg/kg to 10 mg/kg. In yet another embodiment, the dose of antibody is 11 mg/kg to 20 mg/kg. The progress of an antibody therapy can be monitored using standard techniques and assays (See, e.g., International Patent Publication No. WO 94/04188).

5.6.16. Small Molecule Therapy

The present invention also encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e, including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The factors to consider in choosing an appropriate dose of a small molecule agent will be understood by the ordinarily skilled physician, veterinarian, or scientist. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

5.6.17. Gene Therapy.

Gene therapy approaches may also be used in accordance with the present invention to modulate the expression of a BCA gene. Gene therapy refers to therapy performed by the administration to a subject of an expressed or expressible nucleic acid. Accordingly, the present invention provides for a method for treating or preventing a BCA-related disorder comprising administering to a patient in need thereof an effective amount of a mammalian expression vector comprising a BCA polynucleotide, or a variant thereof. In further embodiments, the polynucleotide encodes a BCA polypeptide, BCA agonist, BCA antagonist, inhibitor of a BCA agonist, inhibitor of a BCA antagonist, or a variant thereof.

Any composition described for administration by gene therapy can also be useful, apart from gene therapy approaches, for in vitro or ex vivo manipulations.

Any of the methods for gene therapy available in the art can be used in accordance with the present invention (See, e.g., Goldspiel et al., 1993, Clinical Pharmacy 12:488-505; Grossman and Wilson, 1993, Curr. Opin. in Genetics and Devel. 3:110-114; Salmons and Gunzberg, 1993, Human Gene Therapy 4:129-141; Morgan and Anderson, 1993, Ann. Rev. Biochem. 62:191-217; Mulligan, 1993, Science 260:926-932; Tolstoshev, 1993, Ann. Rev. Pharmacol. Toxicol. 32:573-596; and Clowes et al., 1994, J. Clin. Invest. 93:644-651; Kiem et al., 1994, Blood 83:1467-1473, each of which is incorporated herein by reference). Gene therapy vectors can be administered to a subject systemically or locally by, for example, intravenous injection (See, e.g., U.S. Pat. No. 5,328,470) or by stereotactic injection (See, e.g., Chen et al., 1994, Proc Natl Acad Sci. 91:3054-57). Synthetic genes, the in vitro or in vivo transcription and translation of which results in the production of a BCA antagonist, for example, may be constructed by techniques well known in the art. For example, antisense, ribozyme, triple helix molecules, and/or recombinant antibodies may be used to target by gene therapy a BCA gene of the invention, resulting in a decrease in the respective BCA gene expression and/or BCA protein levels. Techniques for the production and use of antisense, ribozyme, and/or triple helix molecules are well known to those of skill in the art, and in accordance with the present invention, can be applied to a nucleotide sequence encoding a BCA polypeptide of the invention.

A pharmaceutical preparation of the gene therapy vector can comprise a gene therapy vector in an acceptable diluent, or can comprise a slow release matrix in which the gene delivery vehicle is embedded. Alternatively, where the complete gene delivery vector can be produced intact from recombinant cells, e.g., retroviral vectors, the pharmaceutical preparation can include one or more cells which produce the gene delivery system.

The present invention encompasses vectors comprising a nucleic acid encoding a BCA polypeptide of the invention, or the complement thereof. In one embodiment, a BCA polynucleotide of the invention to be introduced for purposes of gene therapy comprises an inducible promoter operably linked to the coding region in the antisense orientation, such that expression of the nucleic acid can be controlled using an appropriate inducer or inhibitor of transcription. In another embodiment, the vector comprises a promoter which expresses the cloned construct constitutively. In a further embodiment, the promoter can be downregulated by a suppressor molecule. Alternatively, the vector comprises a promoter, such that an inducing molecule initiates or increases expression of the cloned antisense BCA polynucleotide. In a preferred embodiment, the vector comprises a cell-specific promoter. In another preferred embodiment, the vector comprises a disease-specific promoter, such that expression is largely limited to diseased tissues or tissues surrounding diseased tissues. In another particular embodiment, a BCA antisense oligonucleotide is placed within a mammalian expression vector such that a BCA antisense construct comprises the entire cDNA sequence.

Gene therapy involves introducing a gene construct to cells in tissue culture or in vivo. Methods for introduction of polynucleotides of the invention to cells in vitro include, but are not limited to, electroporation, lipofection, calcium phosphate-mediated transfection, and viral infection. Usually, the method of transfer includes the transfer of a selectable marker to the cells, after which the cells are placed under selection to isolate the cells which have taken up and express the transferred gene. The transfected cells then an be administered to a subject.

An expression construct can be delivered directly into a subject. In one embodiment, the polynucleotides of the invention can be injected directly into the target tissue or cell derivation site. Alternatively, a subject's cells are first transfected with an expression construct in vitro, after which the transfected cells are administered back into the subject (i.e., ex vivo gene therapy). Accordingly, the polynucleotides of the invention can be delivered in vivo or ex vivo to target cells. Several methods have been developed for delivering the polynucleotides of the invention to target cells or target tissues. Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject. In another embodiment, the polynucleotides of the invention can be introduced into the target tissue as an implant, for example, in a polymer formulation (See e.g., U.S. Pat. No. 5,702,717). In another embodiment, the polynucleotides of the invention can be targeted to the desired cells or tissues.

In one embodiment, a nucleic acid of the invention is administered to inhibit BCA activity using gene therapy.

In a particular embodiment, a vector is introduced in vivo such that it is taken up by a cell and directs the transcription of an antisense BCA nucleic acid of the invention. Such a vector can remain episomal or can become chromosomally integrate. Expression vectors can be plasmid, viral, or others known in the art, that can be used to replicate and/or express the cloned nucleotide sequence encoding a BCA antisense polynucleotide in a target mammalian cell. A variety of expression vectors useful for introducing into cells the polynucleotides of the inventions are well known in the art (See, e.g., Promega™ catalogue, 2001; Stratagene™ catalogue, 2001). Expression constructs can be introduced into target cells and/or tissues of a subject using vectors which include, but are not limited to adenovirus, adeno-associated virus, retrovirus and herpes virus vectors, in addition to other particles that introduce DNA into cells, such as liposomes.

A polynucleotide of the invention can be expressed using any promoter known in the art capable of expression in mammalian, preferably human cells. Such promoters can be inducible or constitutive. These promoters include, but are not limited to, a casein promoter (Cerdan et al., 1998, “Accurate spatial and temporal transgene expression driven by a 3.8-kilobase promoter of the bovine beta-casein gene in the lactating mouse mammary gland”, Mol Reprod Dev 49(3):236-45), whey acid promoter (Doppler et al., 1991, “Lactogenic hormone and cell type-specific control of the whey acidic protein gene promoter in transfected mouse cells”, Mol Endocrinol 5:1624-1632), SV40 early promoter region (Bernoist and Chambon, 1981, Nature 290:304-310), the promoter compriseed in the 3′ long terminal repeat of Rous sarcoma virus (Yamamoto et al., 1980, Cell 22:787-797), the herpes thymidine kinase promoter (Wagner et al., 1981, Proc Natl Acad Sci. 78:1441-1445), and the regulatory sequences of the metallothionein gene (Brinster et al., 1982, Nature 296:39-42).

In one embodiment in which recombinant cells are used in gene therapy, nucleotides complementary to polynucleotides encoding polypeptides of the invention are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (See, e.g., PCT Publication WO 94/08598; Stemple and Anderson, 1992, Cell 71:973-985; Pittelkow and Scott, 1986, Mayo Clinic Proc. 61:771; Rheinwald, 1980, Meth Cell Bio. 21A:229).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

A nucleic acid fragment encoding a biologically active portion of a polypeptide of the invention can be prepared by isolating a portion of any of SEQ ID NO: 1, 3 (or GenBank Accession No. AW225336), 5 (or GenBank Accession No. AW225339), 7 (or GenBank Accession No. AW225341), 9 (or GenBank Accession No. AW225337), 11 (or GenBank Accession No. AW225337), or 13 (or GenBank Accession No. AW225340}, expressing the encoded portion of the polypeptide protein (e.g., by recombinant expression in vitro) and assessing the activity of the encoded portion of the polypeptide.

In another embodiment, an antisense BCA polynucleotide comprises an appended group such as a peptide (e.g., for targeting host cell receptors in vivo), or an agent that facilitates transport across the cell membrane (See, e.g., Letsinger et al., 1989, Proc Natl Acad Sci. 86:6553-6556; Lemaitre et al., 1987, Proc Natl Acad Sci. 84:648-652; PCT Publication No. WO 88/09810) or the blood-brain barrier (See, e.g., PCT Publication No. WO 89/10134). In another embodiment, an antisense BCA polynucleotide can be modified with hybridization-triggered cleavage agents (See, e.g., Krol et al., 1988, BioTechniques 6:958-976) or intercalating agents (See, e.g., Zon, 1988, Pharm Res. 5:539-549). To this end, an antisense BCA polynucleotide may be conjugated to another molecule, e.g., a peptide, hybridization triggered cross-linking agent, transport agent, or hybridization-triggered cleavage agent.

Any type of plasmid, cosmid, YAC or viral vector can be used to prepare the recombinant construct. Alternatively, vectors can be used which selectively target a tissue or cell type, e.g., viruses which infect breast cells. Further specificity can be realized by using a tissue-specific or cell-specific promoter in the expression vector.

In a specific embodiment, an expression vector is administered directly in vivo, where the vector is expressed to produce the encoded product. This can be accomplished by any of numerous methods known in the art, e.g., by placing a nucleic acid of the invention in an appropriate expression vector such that, upon administration, the vector becomes intracellular and expresses a BCA antisense oligonucleotide. Such vectors can be internalized by using, for example, a defective or attenuated retroviral vector or other viral vectors that can infect mammalian cells (See, e.g., U.S. Pat. No. 4,980,286).

Alternatively, an expression construct comprising a nucleic acid of the invention can be injected directly into a target tissue as naked DNA. In another embodiment, an expression construct comprising a nucleic acid of the invention can be introduced intracellularly using microparticle bombardment, for example, by using a Biolistic gene gun (Dupont™). In another embodiment, an expression construct comprising a nucleic acid of the invention can be coated with lipids, or cell-surface receptors, or transfecting agents, such that encapsulation in liposomes, microparticles, or microcapsules facilitates access to target tissues and/or entry into target cells. In yet another embodiment, an expression construct comprising a nucleic acid of the invention is linked to a polypeptide that is internalized in a subset of cells or is targeted to a particular cellular compartment. In a further embodiment, the linked polypeptide is a nuclear targeting sequence which targets the vector to the cell nucleus. In another further embodiment, the linked polypeptide is a ligand that is internalized by receptor-mediated endocytosis in cells expressing the respective receptor for the ligand (See, e.g., Wu and Wu, 1987, J. Biol. Chem. 262:4429-4432).

In another embodiment, nucleic acid-ligand complexes can be formed such that the ligand comprises a fusogenic viral peptide which disrupts endosomes, thereby allowing the nucleic acid to avoid lysosomal degradation. In another embodiment, a nucleic acid of the invention can be targeted in vivo via a cell-specific receptor resulting in cell-specific uptake and expression (See, e.g., International Patent Publications WO 92/06180, WO 92/22635, WO 92/20316, WO 93/14188, and WO 93/2022. In yet another embodiment, a nucleic acid of the invention is introduced intracellularly and, by homologous recombination, can transiently or stably be incorporated within the host cell DNA, which then allows for its expression, (Koller and Smithies, 1989, Proc Natl Acad Sci. 86:8932-8935; Zijlstra et al., 1989, Nature 342:435-438).

In one embodiment, viral vectors are used that comprise nucleic acids encoding compounds that activate cytokine receptors (i.e., cytokines or antibodies), or compounds that activate molecules expressed on activated immune cells (See, e.g., Miller et al., 1993, Meth. Enzymol. 217:581-599). In a specific embodiment, a viral vector that comprises polynucleotides encoding 4-1BB ligand, or anti-4-1BB immunoglobulin, and/or IL-12 are used. For example, a retroviral vector can be used in which sequences not necessary for packaging of the viral genome and integration into host cell DNA have been deleted, and polynucleotides encoding 4-1BB ligand, or anti-4-1BB immunoglobulin, or IL-12 are cloned into the vector, thereby facilitating delivery of the transgene into a subject. Greater detail about retroviral vectors is available in Boesen et al., 1994, Biotherapy 6:291-302, which describes the use of a retroviral vector to deliver the mdr1 gene to hematopoietic stem cells.

Other viral vectors can be used for gene therapy approaches in accordance with the invention. For example, adenoviruses are useful for delivering gene constructs to respiratory epithelia. Other targets for adenovirus-based delivery systems are the liver, the central nervous system, endothelial cells, and muscle cells. Moreover, adenoviruses are able to infect non-dividing cells (See, e.g., Rosenfeld et al., 1991, Science 252:431-434; Rosenfeld et al., 1992, Cell 68:143-155; Mastrangeli et al., 1993, J. Clin. Invest. 91:225-234; Kozarsky and Wilson, 1993, Curr. Opin. Genetics Develop. 3:499-503; Bout et al., 1994, Human Gene Therapy 5:3-10; PCT Publication No. WO 94/12649; and Wang et al., 1995, Gene Therapy 2:775-783).

Adeno-associated virus can also be used in accordance with the gene therapy approaches of the present invention (See, e.g., Walsh et al., 1993, Proc. Soc. Exp. Biol. Med. 204:289-300; U.S. Pat. No. 5,436,146).

In this embodiment, the nucleic acid is introduced into a cell prior to administration in vivo of the resulting recombinant cell. Such introduction can be carried out by any method known in the art, including, but not limited to, transfection, electroporation, microinjection, infection with a viral or bacteriophage vector comprising the polynucleotides, cell fusion, chromosome-mediated gene transfer, microcell-mediated gene transfer, and spheroplast fusion. Numerous techniques are known in the art for the introduction of foreign genes into cells (See, e.g., Maniatis et al., 1989; Current Protocols in Molecular Biology, John Wiley & Sons, 2000; Loeffler and Behr, 1993, Meth. Enzymol. 217:599-618; Cohen et al., 1993, Meth. Enzymol. 217:618-644; Cline, 1985, Pharmacol. Ther. 29:69-92) and can be used in accordance with the present invention. In a preferred embodiment, the technique stably transfers a nucleic acid of the invention to a target cell, such that the nucleic acid is inherited by the cell's progeny.

The resulting recombinant cells can be delivered to a subject by various methods known in the art, and the skilled artisan would appreciate appropriate modes of administration. For example, intravenous administration may be the preferred mode of administration for recombinant hematopoietic stem cells. Similarly, the number of recombinant cells to be administered to a subject can be determined by one skilled in the art, and would include a consideration of factors such as the desired effect, the disease state, and the mode of administration.

Cells into which a nucleic acid of the invention can be introduced for purposes of gene therapy include, but are not limited to, epithelial cells, endothelial cells, keratinocytes, fibroblasts, muscle cells, hepatocytes, blood cells (e.g., B lymphocytes, T lymphocytes, eosinophils, granulocytes, macrophages, megakaryocytes, monocytes, neutrophils), stem cells or progenitor cells (e.g., undifferentiated cells obtained from adipose, bone marrow, blood, fetal liver, and umbilical cord (See, e.g., Rheinwald, 1980, Meth. Cell Bio. 21A:229; International Publication No. WO 94/08598; Pittelkow and Scott, 1986, Mayo Clinic Proc. 61:771; and Stemple and Anderson, 1992, Cell 71:973-985). The cells used for introduction of a nucleic acid of the invention can be autologous or non-autologous. In a preferred embodiment, the cells used for gene therapy are autologous to the subject.

One skilled in the art will appreciate that many different promoters can be used to drive expression of a BCA antisense construct. In one embodiment, the promoter comprises hormone-sensitive elements. For example, a promoter comprising an androgen-sensitive enhancer would be activated to greater degree in androgen-producing cells or adjacent tissues. Such an expression construct may be beneficial for targeting tissues secreting abnormally high levels of androgen. In another embodiment, the promoter comprises elements of a fibroblast-specific promoter. In a further embodiment, the fibroblast-specific promoter comprises promoter elements from synovial fibroblasts. Alternatively, the promoter comprises elements of promoters that are activated in aggressive rheumatoid arthritis synovial fibroblasts. In a particular embodiment, the promoter comprises a portion of a BCA promoter. In a non-limiting example, a viral vector is used in which the viral promoter is replaced fully, or in part, with at least parts of a BCA promoter. Such an expression construct would more specifically be expressed in BCA-expressing cells, and higher expression of a BCA antisense oligonucleotide would occur in cells expressing above-normal levels of BCA.

Gene therapy approaches may also be used in accordance with the present invention to inhibit BCA. For example, ribozyme and triple helix molecules may be used to target a BCA gene products, resulting in a decrease in BCA protein. Techniques for the production and use of antisense ribozyme and/or triple helix molecules are well known to those of skill in the art and can be designed with respect to the nucleotide sequence encoding the amino acid sequence of BCA, also known in the art.

5.6.17.1. Antisense Gene Therapy

Antisense approaches to gene therapy involve the use of riboprobes that may hybridize to a portion of the target mRNA. The skilled artisan will recognize that absolute complementarity is not required, such that some degree of mismatch can result in, at least, transitory duplex formation. In one non-limiting example, the antisense riboprobe binds to the target mRNA transcript and prevents its translation.

Riboprobes that are complementary to the 5′ untranslated sequences, up to and including the AUG initiation codon, can be used effectively to inhibit translation of a BCA mRNA. Additionally, riboprobes complementary to the 3′ untranslated sequences of mRNAs also can be effective at inhibiting BCA mRNA translation (See, e.g., Wagner, 1994, Nature 372:333-335). Moreover, antisense riboprobes complementary to mRNA coding regions can be used in accordance with the invention.

Preferably, in vitro studies are performed to assess the ability of an antisense riboprobe to inhibit gene expression. These studies typically use controls which distinguish between antisense-mediated inhibition of gene expression and nonspecific biological effects of riboprobes. Preferably, these studies compare antisense-mediated changes in the levels of the target RNA or target protein with levels of an internal control RNA or protein.

In one embodiment, a recombinant DNA construct that has a BCA antisense riboprobe under the control of a pol III or pol II promoter is used to generate BCA antisense riboprobes in a cell. The use of such a construct to transfect target cells in the subject can result in the transcription of sufficient amounts of a BCA riboprobe to reduce or inhibit BCA mRNA and/or protein expression. Low transfection rates or low transcription activity of the DNA construct can nevertheless generate sufficient BCA antisense molecules to demonstrate clinical effectiveness.

In another embodiment, a BCA antisense polynucleotide is cloned into an expression vector, preferably a mammalian expression vector. In a specific embodiment, the BCA antisense polynucleotide comprises the sequence of a full-length BCA DNA. In another specific embodiment, the BCA antisense polynucleotide comprises the sequence of a 5′ untranslated region, which optionally can include the sequence, AUG, indicating the start of the coding region.

In one embodiment the BCA antisense polynucleotide is about 50 bp in length. In a particular embodiment, the BCA antisense polynucleotide comprises the sequence from nucleotides 1-50, 51-100, 101-150, 151-200, 201-250, 251-300, 301-350, 351-400, 401-450, 451-500, 501-550, 551-600, 601-650, 651-700, 701-750, 751-800, 801-850, 851-900, 901-950, 951-1000, 1001-1050, 1051-1100, 1101-1150, 1151-1200, 1201-1250, 1251-1300, 1301-1350, or 1351-1400 of SEQ ID NOs:1, 3 (or GenBank Accession No. AW225336), 5 (or GenBank Accession No. AW225337), 7 (or GenBank Accession No. AW225341), 9 (or GenBank Accession No. AW225337), 11 (or GenBank Accession No. AW225338), and 13 (or GenBank Accession No. AW225340).

In another embodiment the BCA antisense polynucleotide is about 100 bp in length. In a particular embodiment, the BCA antisense polynucleotide comprises the sequence from nucleotides 1-100, 101-200, 201-300, 301-400, 401-500, 501-600, 601-700, 701-800, 801-900, 901-1000, 1001-1100, 1101-1200, 1201-1300, or 1301-1400 of SEQ ID NOs:1, 3 (or GenBank Accession No. AW225336), 5 (or GenBank Accession No. AW225337), 7 (or GenBank Accession No. AW225341), 9 (or GenBank Accession No. AW225337), 11 (or GenBank Accession No. AW225338), and 13 (or GenBank Accession No. AW225340).

In another embodiment the BCA antisense polynucleotide is about 200 bp in length. In a particular embodiment, the BCA antisense polynucleotide comprises the sequence from nucleotides 1-200, 201-400, 401-600, 601-800, 801-1000, 1001-1200, or 1201-1400 of SEQ ID NOs:1, 3 (or GenBank Accession No. AW225336), 5 (or GenBank Accession No. AW225337), 7 (or GenBank Accession No. AW225341), 9 (or GenBank Accession No. AW225337), 11 (or GenBank Accession No. AW225338), and 13 (or GenBank Accession No. AW225340).

In another embodiment the BCA antisense polynucleotide is about 400 bp in length. In a particular embodiment, the BCA antisense polynucleotide comprises the sequence from nucleotides 1-400, 401-800, 801-1200, or 1201-1600 of SEQ ID NOs:1, 3 (or GenBank Accession No. AW225336), 5 (or GenBank Accession No. AW225337), 7 (or GenBank Accession No. AW225341), 9 (or GenBank Accession No. AW225337), 11 (or GenBank Accession No. AW225338), and 13 (or GenBank Accession No. AW225340). In a specific embodiment, the BCA antisense polynucleotide comprises the sequence from nucleotides 100-500, 501-900, 901-1300, or 1301-1700 of SEQ ID NOs:1, 3 (or GenBank Accession No. AW225336), 5 (or GenBank Accession No. AW225337), 7 (or GenBank Accession No. AW225341), 9 (or GenBank Accession No. AW225337), 11 (or GenBank Accession No. AW225338), and 13 (or GenBank Accession No. AW225340). In yet another specific embodiment, the BCA antisense polynucleotide comprises the sequence from nucleotides 200-600, 601-1000, or 1001-1400 of SEQ ID NOs:1, 3 (or GenBank Accession No. AW225336), 5 (or GenBank Accession No. AW225337), 7 (or GenBank Accession No. AW225341), 9 (or GenBank Accession No. AW225337), 11 (or GenBank Accession No. AW225338), and 13 (or GenBank Accession No. AW225340).

In another embodiment, antisense polynucleotides of the invention are cloned into a vector, which is designed to target the vector (and thereby target expression of the antisense riboprobe) to specific tissues or cell-types. For example, an antisense riboprobe can be linked to peptides or antibodies that specifically bind receptors or antigens expressed on the target cell surface, thereby targeting the vector to cells) can be administered.

In another embodiment, the vector comprises a promoter that is more highly activated in diseased cells or tissues, as compared to normal cells or tissues.

A preferred approach to achieve intracellular concentrations of the antisense sufficient to suppress translation of endogenous mRNAs involves the use of a recombinant DNA construct in which the antisense oligonucleotide is placed under the control of a strong pol III or pol II promoter. The use of such a construct to transfect target cells in a patient will result in the transcription of sufficient amounts of single stranded RNAs that can form complementary base pairs with the endogenous BCA1-7 gene transcripts and thereby prevent translation of the BCA1-7 gene mRNA. For example, a vector can be introduced in vivo such that the vector is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290:304-310), the promoter compriseed in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), and the regulatory sequences of the metallothionein gene (Brinster et al., 1982, Nature 296:39-42). Any type of plasmid, cosmid, YAC (yeast artificial chromosome), or viral vector can be used to prepare the recombinant DNA construct which can be introduced directly into the tissue site. Alternatively, viral vectors can be used which selectively infect the desired tissue.

5.6.17.2. Ribozyme Therapy

The invention also encompasses ribozymes. Ribozymes are enzymatic RNA molecules capable of catalyzing the specific cleavage of a single-stranded nucleic acid, such as an mRNA (See, e.g., Rossi, 1994, Current Biology 4:469-471). The mechanism of a trans-acting ribozyme action involves sequence-specific hybridization of the ribozyme molecule to a complementary target, followed by an endonucleolytic cleavage. The composition of ribozyme molecules include one or more sequences complementary to the target gene mRNA, and catalytic sequences responsible for mRNA cleavage (See, e.g., U.S. Pat. No. 5,093,246 which is incorporated by reference in its entirety). Thus, ribozymes, e.g., hammerhead ribozymes (Haselhoff and Gerlach, 1988, Nature 334:585-591), can be used to catalytically cleave mRNA transcripts thereby inhibiting the expression of a protein encoded by a particular mRNA. A trans-acting ribozyme having specificity for a polynucleotide encoding a polypeptide of the invention can be designed based upon the nucleotide sequence of the polynucleotides of the invention. Accordingly, in one embodiment, an engineered hammerhead motif ribozyme molecule specifically and efficiently catalyzes endonucleolytic cleavage of RNA sequences encoding a BCA polypeptide of the invention.

In another embodiment, an mRNA encoding a polypeptide of the invention is used to select a catalytic RNA having a specific ribonuclease activity from a pool of RNA molecules (See, e.g., Bartel and Szostak, 1993, Science 261:1411-1418).

Specific ribozyme cleavage sites within a potential RNA target are identified by scanning the molecule of interest for ribozyme cleavage sites, which include the sequences GUA, GUU and GUC. Once identified, short RNA sequences of approximately 15 to 20 ribonucleotides corresponding to a cleavage site of a target gene are evaluated for predicted structural features, such as secondary structure, that may make the oligonucleotide suitable. The suitability of candidate sequences also can be evaluated by testing their ability to hybridize with complementary oligonucleotides, using for example, ribonuclease protection assays.

In one embodiment, a ribozyme in the form of an antisense riboprobe is generated from a mammalian expression vector. In another embodiment, a ribozyme in the form of an oligonucleotide administered directly to the patient. In a further embodiment, the ribozyme is administered systemically. In another further embodiment, the ribozyme is administered directly to the cells or tissue, in vivo or ex vivo.

The ribozymes of the present invention also include RNA endoribonucleases, such as the ribozymes which occurs naturally in Tetrahymena thermophila (also known as the IVS, or L-19 IVS RNA) and has been extensively described (Zaug et al., 1984, Science 224:574-578; Been and Cech, 1986, Cell 47:207-216; Zaug and Cech, 1986, Science 231:470-475; Zaug et al., 1986, Nature 324:429-433; published International Patent Publication No. WO 88/04300). These ribozymes have an 8 bp active site which hybridizes to a target RNA sequence to cause cleavage of the target RNA. Accordingly, the invention encompasses ribozymes that target active sites comprising 8 bp, which are present in a BCA gene.

As discussed for antisense approaches, supra, the ribozymes of the invention can be composed of modified oligonucleotides (e.g. for improved stability or targeting) and should be delivered to cells that express a BCA gene in vivo. A preferred method of delivery involves using a DNA construct “encoding” the ribozyme under the control of a strong constitutive pol III or pol II promoter, so that transfected cells will produce sufficient quantities of the ribozyme to cause degradation of an endogenous BCA mRNA and thereby inhibit translation. Because ribozymes unlike antisense molecules, are catalytic, a relatively low intracellular concentration is required for efficiency.

Ribozymes of the invention can be prepared by any method known in the art for the synthesis of DNA and RNA molecules. For example, chemical synthesis can be achieved by synthesizing oligodeoxyribonucleotides and oligoribonucleotides using solid phase phosphoramidite chemical synthesis. Alternatively, ribozyme polynucleotides can be generated by in vitro or in vivo transcription of DNA sequences. Such DNA sequences can be incorporated into a wide variety of vectors which incorporate suitable RNA polymerase promoters such as the T7 or SP6 polymerase promoters. Alternatively, antisense cDNA constructs can be introduced stably into cell lines, such that the synthesize ribozymes are expressed constitutively or inducibly, depending on the promoter used.

5.6.17.3. Triple Helix Therapy

The invention also encompasses polynucleotides which form triple helical structures. For example, expression of a polypeptide of the invention can be inhibited by targeting nucleotide sequences complementary to the regulatory region of the gene encoding the polypeptide (e.g., the promoter and/or enhancer) to form triple helical structures that prevent transcription of the gene in target cells (See, e.g., Helene, 1991, Anticancer Drug Des. 6:569-84; Helene, 1992, Ann NY Acad Sci. 660:27-36; Maher, 1992, Bioassays 14:807-815).

Polynucleotides to be used to inhibit transcription by triple helix formation can be single stranded oligonucleotides. The base composition of these oligonucleotides can be designed to promote triple helix formation via Hoogsteen base pairing rules, preferably with long stretches of purines or pyrimidines on one strand of the duplex. Nucleotide sequences can be pyrimidine-based thereby resulting in TAT and CGC+ triplets across the three associated strands of the resulting triple helix. The pyrimidine-rich molecules provide base complementarity to a purine-rich region of a single strand of the duplex in a parallel orientation to that strand. Purine-rich polynucleotides also can be chosen, for example, comprising a stretch of guanine residues. These molecules can form a triple helix with a DNA duplex that is rich in GC pairs, in which most of the purine residues are located on a single strand of the targeted duplex, resulting in GGC triplets across the three strands in the triplex.

Additionally, the number of potential sequences that can be targeted for triple helix formation can be increased by creating a “switchback” polynucleotide. Switchback molecules are synthesized in an alternating 5′-3′, 3′-5′ manner, such that the molecule first hybridizes with one strand of a duplex, followed by hybridization with another strand, thus eliminating the requirement for a stretch of purines or pyrimidines on one strand of a duplex.

Ribozyme and triple helix molecules of the invention can be prepared by any method known in the art for the synthesis of DNA or RNA molecules (e.g., oligodeoxyribonucleotides or oligoribonucleotides). Such methods include, for example, solid phase phosphoramidite chemical synthesis. For further examples of methods of synthesis, see Section 5.6.14 regarding methods for synthesis of antisense oligonucleotides, supra.

These oligonucleotides can be administered directly, for example, via injection. Alternatively, RNA molecules can be generated in vitro or in vivo by transcription of DNA sequences. Such DNA sequences may be incorporated into a wide variety of vectors known in the art that feature a suitable RNA polymerase promoter such as, for example, a T7 or SP6 polymerase promoter. In a preferred embodiment, a breast-cell specific promoter is used to produce an expression vector comprising a polynucleotide of the invention. In another preferred embodiment, a cancer-specific promoter is used to produce an expression vector comprising a polynucleotide of the invention. In a specific embodiment, a whey acidic promoter is used to produce an expression vector comprising a polynucleotide of the invention. In another specific embodiment, a casein promoter is used to produce an expression vector comprising a polynucleotide of the invention. In another specific embodiment, a probasin promoter is used to produce an expression vector comprising a polynucleotide of the invention (See e.g., Zhang et al., 2000, “A small composite probasin promoter confers high levels of prostate-specific gene expression through regulation by androgens and glucocorticoids in vitro and in vivo”, Endocrinology 141:4698-4710). Also contemplated is use of tetracycline inducible vectors (available, e.g., from CLONTECH) and adeno-based vectors. Also intended to be within the scope of the invention is use of any heterologous promoter indicated above.

5.6.17.4. Antibody Therapy

In one embodiment, polynucleotides comprising sequences encoding antibodies that bind to a BCA are administered via gene therapy. In a particular embodiment, recombinant cells are used that comprise polynucleotides encoding antibodies to BCA polypeptides of the invention. The gene construct is expressed such that the recombinant antibody is secreted or expressed on the cell surface. The recombinant cells are then administered in vivo for therapeutic effect.

Antibodies of the invention, including antibodies conjugated to therapeutic moieties, can be administered to an individual alone or in combination with a chemotherapeutic drug, cytotoxic factor, and/or cytokine. In one embodiment, an antibodies directed to a BCA polypeptide is administered first, followed by chemotherapeutic drug, cytotoxic factor, and/or cytokine within 24 hours. The treatment cycle can be repeated if warranted by the clinical response of the patient. Furthermore, the antibody, chemotherapeutic drug, cytotoxic factor, and/or cytokine can be administered via separate routes, such as for example, by intravenous and intramuscular administration. Cytotoxic factors include, but are not limited to, TNF-α, TNF-β, IL-1, IFN-γ, and IL-2. Chemotherapeutic drugs include, but are not limited to, 5-fluorouracil (5FU), vinblastine, actinomycin D, etoposide, cisplatin, methotrexate, and doxorubicin. Cytokines include, but are not limited to, IL-2, IL-3, IL-4, IL-5, IL-6, IL-7, IL-8, IL-9, IL-10, and IL-12.

5.6.17.5. Vaccine Therapy.

The polynucleotides of the invention, including variants thereof, cin be used as vaccines, e.g., by genetic immunization. Genetic immunization is particularly advantageous as it stimulates a cytotoxic T-cell response but does not utilize live attenuated vaccines, which can revert to a virulent form and infect the host causing the very infection sought to be prevented. As used herein, genetic immunization comprises inserting the nucleotides of the invention into a host, such that the nucleotides are taken up by cells of the host and the proteins encoded by the nucleotides are translated. These translated proteins are then either secreted or processed by the host cell for presentation to immune cells and an immune reaction is stimulated. Preferably, the immune reaction is a cytotoxic T cell response, however, a humoral response or macrophage stimulation is also useful in preventing future infections. The skilled artisan will appreciate that there are various methods for introducing foreign nucleotides into a host animal and subsequently into cells for genetic immunization, for example, by intramuscular injection of about 50 mg of plasmid DNA encoding the proteins of the invention solubilized in 50 ml of sterile saline solution, with a suitable adjuvant (See, e.g., Weiner and Kennedy, 1999, Scientific American 7:50-57; Lowrie et al., 1999, Nature 400:269-271).

5.6.18. Combination Therapies

The administration of a BCA antagonist can potentiate the effect of anti-cancer agents. In a preferred embodiment, the invention further encompasses the use of combination therapy to prevent or treat cancer.

In one embodiment, breast cancer can be treated with a pharmaceutical composition comprising a BCA antagonist in combination with 5-fluorouracil, cisplatin, docetaxel, doxorubicin, Herceptin®, gemcitabine (Seidman, 2001, “Gemcitabine as single-agent therapy in the management of advanced breast cancer”, Oncology 15:11-14), IL-2, paclitaxel, and/or VP-16 (etoposide).

In another embodiment, prostate cancer can be treated with a pharmaceutical composition comprising a BCA antagonist in combination with paclitaxel, docetaxel, mitoxantrone, and/or an androgen receptor antagonist (e.g., flutamide).

In another embodiment, leukemia can be treated with a pharmaceutical composition comprising a BCA antagonist in combination with fludarabine, cytosine arabinoside, gemtuzumab (MYLOTARG), daunorubicin, methotrexate, vincristine, 6-mercaptopurine, idarubicin, mitoxantrone, etoposide, asparaginase, prednisone and/or cyclophosphamide. As another example, myeloma can be treated with a pharmaceutical composition comprising a BCA antagonist in combination with dexamethasone.

In another embodiment, melanoma can be treated with a pharmaceutical composition comprising a BCA antagonist in combination with dacarbazine.

In another embodiment, colorectal cancer can be treated with a pharmaceutical composition comprising a BCA antagonist in combination with irinotecan.

In another embodiment, lung cancer can be treated with a pharmaceutical composition comprising a BCA antagonist in combination with paclitaxel, docetaxel, etoposide and/or cisplatin.

In another embodiment, non-Hodgkin's lymphoma can be treated with a pharmaceutical composition comprising a BCA antagonist in combination with cyclophosphamide, CHOP, etoposide, bleomycin, mitoxantrone and/or cisplatin.

In another embodiment, gastric cancer can be treated with a pharmaceutical composition comprising a BCA antagonist in combination with cisplatin.

In another embodiment, pancreatic cancer can be treated with a pharmaceutical composition comprising a BCA antagonist in combination with gemcitabine.

These combination therapies can also be used to prevent cancer, prevent the recurrence of cancer, or prevent the spread or metastasis or cancer.

Combination therapy also includes, in addition to administration of a BCA antagonist, the use of one or more molecules, compounds or treatments that aid in the prevention or treatment of cancer (i.e., cancer therapeutics), which molecules, compounds or treatments includes, but is not limited to, chemoagents, immunotherapeutics, cancer vaccines, anti-angiogenic agents, cytokines, hormone therapies, gene therapies, and radiotherapies.

In one embodiment, one or more chemoagents, in addition to a BCA antagonist, is administered to treat a cancer patient. A chemoagent (or “anti-cancer agent” or “anti-tumor agent” or “cancer therapeutic”) refers to any molecule or compound that assists in the treatment of tumors or cancer. Examples of chemoagents contemplated by the present invention include, but are not limited to, cytosine arabinoside, taxoids (e.g., paclitaxel, docetaxel), anti-tubulin agents (e.g., paclitaxel, docetaxel, epothilone B, or its analogues), macrolides (e.g., rhizoxin) cisplatin, carboplatin, adriamycin, tenoposide, mitozantron, discodermolide, eleutherobine, 2-chlorodeoxyadenosine, alkylating agents (e.g., cyclophosphamide, mechlorethamine, thioepa, chlorambucil, melphalan, carmustine (BSNU), lomustine (CCNU), cyclothosphamide, busulfan, dibromomannitol, streptozotocin, mitomycin C, and cis-dichlorodiamine platinum (II) (DDP) cisplatin, thio-tepa), antibiotics (e.g., dactinomycin (formerly actinomycin), bleomycin, mithramycin, anthramycin), antimetabolites (e.g., methotrexate, 6-mercaptopurine, 6-thioguanine, cytarabine, flavopiridol, 5-fluorouracil, fludarabine, gemcitabine, dacarbazine, temozolamide), asparaginase, Bacillus Calmette and Guerin, diphtheria toxin, hexamethylmelamine, hydroxyurea, LYSODREN®, nucleoside analogues, plant alkaloids (e.g., Taxol, paclitaxel, camptothecin, topotecan, irinotecan (CAMPTOSAR, CPT-11), vincristine, vinca alkyloids such as vinblastine), podophyllotoxin (including derivatives such as epipodophyllotoxin, VP-16 (etoposide), VM-26 (teniposide)), cytochalasin B, colchine, gramicidin D, ethidium bromide, emetine, mitomycin, procarbazine, mechlorethamine, anthracyclines (e.g., daunorubicin (formerly daunomycin), doxorubicin, doxorubicin liposomal), dihydroxyanthracindione, mitoxantrone, mithramycin, actinomycin D, procaine, tetracaine, lidocaine, propranolol, puromycin, anti-mitotic agents, abrin, ricin A, pseudomonas exotoxin, nerve growth factor, platelet derived growth factor, tissue plasminogen activator, aldesleukin, allutamine, anastrozle, bicalutamide, biaomycin, busulfan, capecitabine, carboplain, chlorabusil, cladribine, cylarabine, daclinomycin, estramusine, floxuridhe, gemcitabine, gosereine, idarubicin, itosfamide, lauprolide acetate, levamisole, lomusline, mechlorethamine, magestrol, acetate, mercaptopurino, mesna, mitolanc, pegaspergase, pentoslatin, picamycin, riuxlmab, campath-1, straplozocin, thioguanine, tretinoin, vinorelbine, or any fragments, family members, or variants thereof, including pharmaceutically acceptable salts thereof. Compositions comprising one or more chemoagents (e.g., FLAG, CHOP) are also contemplated by the present invention. FLAG comprises fludarabine, cytosine arabinoside (Ara-C) and G-CSF. CHOP comprises cyclophosphamide, vincristine, doxorubicin, and prednisone.

In one embodiment, the chemoagent is gemcitabine at a dose ranging from 100 to 1000 mg/m²/cycle. In one embodiment, the chemoagent is dacarbazine at a dose ranging from 200 to 4000 mg/m²/cycle. In a preferred embodiment, the dose ranges from 700 to 1000 mg/m²/cycle. In another embodiment, the chemoagent is fludarabine at a dose ranging from 25 to 50 mg/m²/cycle. In another embodiment, the chemoagent is cytosine arabinoside (Ara-C) at a dose ranging from 200 to 2000 mg/m²/cycle. In another embodiment, the chemoagent is docetaxel at a dose ranging from 1.5 to 7.5 mg/kg/cycle. In another embodiment, the chemoagent is paclitaxel at a dose ranging from 5 to 15 mg/kg/cycle. In yet another embodiment, the chemoagent is cisplatin at a dose ranging from 5 to 20 mg/kg/cycle. In yet another embodiment, the chemoagent is 5-fluorouracil at a dose ranging from 5 to 20 mg/kg/cycle. In yet another embodiment, the chemoagent is doxorubicin at a dose ranging from 2 to 8 mg/kg/cycle. In yet another embodiment, the chemoagent is epipodophyllotoxin at a dose ranging from 40 to 160 mg/kg/cycle. In yet another embodiment, the chemoagent is cyclophosphamide at a dose ranging from 50 to 200 mg/kg/cycle. In yet another embodiment, the chemoagent is irinotecan at a dose ranging from 50 to 75, 75 to 100, 100 to 125, or 125 to 150 mg/m²/cycle. In yet another embodiment, the chemoagent is vinblastine at a dose ranging from 3.7 to 5.4, 5.5 to 7.4, 7.5 to 11, or 11 to 18.5 mg/m²/cycle. In yet another embodiment, the chemoagent is vincristine at a dose ranging from 0.7 to 1.4, or 1.5 to 2 mg/m²/cycle. In yet another embodiment, the chemoagent is methotrexate at a dose ranging from 3.3 to 5, 5 to 10, 10 to 100, or 100 to 1000 mg/m²/cycle.

In a preferred embodiment, the invention further encompasses the use of low doses of chemoagents when administered as part of a BCA antagonist treatment regimen. For example, initial treatment with a BCA antagonist increases the sensitivity of a tumor to subsequent challenge with a dose of chemoagent, which dose is near or below the lower range of dosages when the chemoagent is administered without a BCA antagonist. In one embodiment, a BCA antagonist and a low dose (e.g., 6 to 60 mg/m²/day or less) of docetaxel are administered to a cancer patient. In another embodiment, a BCA antagonist and a low dose (e.g., 10 to 135 mg/m²/day or less) of paclitaxel are administered to a cancer patient. In yet another embodiment, a BCA antagonist and a low dose (e.g., 2.5 to 25 mg/m²/day or less) of fludarabine are administered to a cancer patient. In yet another embodiment, a BCA antagonist and a low dose (e.g., 0.5 to 1.5 g/m²/day or less) of cytosine arabinoside (Ara-C) are administered to a cancer patient.

The invention, therefore, contemplates the use of one or more BCA antagonists, which is administered prior to, subsequently, or concurrently with low doses of chemoagents, for the prevention or treatment of cancer.

In one embodiment, the chemoagent is gemcitabine at a dose ranging from 10 to 100 mg/m²/cycle.

In one embodiment, the chemoagent is cisplatin, e.g., PLATINOL or PLATINOL-AQ (Bristol Myers), at a dose ranging from 5 to 10, 10 to 20, 20 to 40, or 40 to 75 mg/m²/cycle. In another embodiment, a dose of cisplatin ranging from 7.5 to 75 mg/m²/cycle is administered to a patient with ovarian cancer. In another embodiment, a dose of cisplatin ranging from 5 to 50 mg/m²/cycle is administered to a patient with bladder cancer.

In another embodiment, the chemoagent is carboplatin, e.g., PARAPLATIN (Bristol Myers), at a dose ranging from 2 to 4, 4 to 8, 8 to 16, 16 to 35, or 35 to 75 mg/m²/cycle. In another embodiment, a dose of carboplatin ranging from 7.5 to 75 mg/m²/cycle is administered to a patient with ovarian cancer. In another embodiment, a dose of carboplatin ranging from 5 to 50 mg/m²/cycle is administered to a patient with bladder cancer. In another embodiment, a dose of carboplatin ranging from 2 to 20 mg/m²/cycle is administered to a patient with testicular cancer.

In another embodiment, the chemoagent is docetaxel, e.g., TAXOTERE (Rhone Poulenc Rorer) at a dose ranging from 6 to 10, 10 to 30, or 30 to 60 mg/m²/cycle.

In another embodiment, the chemoagent is paclitaxel, e.g., TAXOL (Bristol Myers Squibb), at a dose ranging from 10 to 20, 20 to 40, 40 to 70, or 70 to 135 mg/kg/cycle.

In another embodiment, the chemoagent is 5-fluorouracil at a dose ranging from 0.5 to 5 mg/kg/cycle.

In another embodiment, the chemoagent is doxorubicin, e.g., ADRIAMYCIN (Pharmacia & Upjohn), DOXIL (Alza), RUBEX (Bristol Myers Squibb), at a dose ranging from 2 to 4, 4 to 8, 8 to 15, 15 to 30, or 30 to 60 mg/kg/cycle.

In another embodiment, a BCA antagonist is administered in combination with one or more immunotherapeutic agents, such as antibodies and immunomodulators, which includes, but is not limited to, Herceptin®, Retuxan®, OvaRex, Panorex, BEC2, IMC-C225, Vitaxin, Campath I/H, Smart M195, LymphoCide, Smart I D10, and Oncolym, rituxan, rituximab, gemtuzumab, or trastuzumab.

In another embodiment, a BCA antagonist is administered in combination with one or more anti-angiogenic agents, which includes, but is not limited to, angiostatin, thalidomide, kringle 5, endostatin, Serpin (Serine Protease Inhibitor) anti-thrombin, 29 kDa N-terminal and a 40 kDa C-terminal proteolytic fragments of fibronectin, 16 kDa proteolytic fragment of prolactin, 7.8 kDa proteolytic fragment of platelet factor-4, a 13-amino acid peptide corresponding to a fragment of platelet factor-4 (Maione et al., 1990, Cancer Res. 51:2077-2083), a 14-amino acid peptide corresponding to a fragment of collagen I (Tolma et al., 1993, J. Cell Biol. 122:497-511), a 19 amino acid peptide corresponding to a fragment of Thrombospondin I (Tolsma et al., 1993, J. Cell Biol. 122:497-511), a 20-amino acid peptide corresponding to a fragment of SPARC (Sage et al., 1995, J. Cell. Biochem. 57:1329-1334), or any fragments, family members, or variants thereof, including pharmaceutically acceptable salts thereof.

Other peptides that inhibit angiogenesis and correspond to fragments of laminin, fibronectin, procollagen, and EGF have also been described (see, e.g., Cao, 1998, Prog Mol Subcell Biol. 20:161-176). Monoclonal antibodies and cyclic pentapeptides, which block certain integrins that bind RGD proteins (i.e., possess the peptide motif Arg-Gly-Asp), have been demonstrated to have anti-vascularization activities (Brooks et al., 1994, Science 254:569-571; Hammes et al., 1996, Nature Medicine 2:529-533). Moreover, inhibition of the urokinase plasminogen activator receptor by receptor antagonists inhibits angiogenesis, tumor growth and metastasis (Min et al., 1996, Cancer Res. 56: 2428-33; Crowley et al., 1993, Proc Natl Acad Sci. 90:5021-25). Use of such anti-angiogenic agents is also contemplated by the present invention.

In another embodiment, a BCA antagonist is administered in combination with a regimen of radiation.

In another embodiment, a BCA antagonist is administered in combination with one or more cytokines, which includes, but is not limited to, lymphokines, tumor necrosis factors, tumor necrosis factor-like cytokines, lymphotoxin-α, lymphotoxinβ, interferon-α, interferonβ, macrophage inflammatory proteins, granulocyte monocyte colony stimulating factor, interleukins (including, but not limited to, interleukin-1, interleukin-2, interleukin-6, interleukin-12, interleukin-15, interleukin-18), OX40, CD27, CD30, CD40 or CD137 ligands, Fas-Fas ligand, 4-1BBL, endothelial monocyte activating protein or any fragments, family members, or variants thereof, including pharmaceutically acceptable salts thereof.

In yet another embodiment, a BCA antagonist is administered in combination with a cancer vaccine. Examples of cancer vaccines include, but are not limited to, autologous cells or tissues, non-autologous cells or tissues, carcinoembryonic antigen, alpha-fetoprotein, human chorionic gonadotropin, BCG live vaccine, melanocyte lineage proteins (e.g., gp100, MART-1/MelanA, TRP-1 (gp75), tyrosinase, widely shared tumor-specific antigens (e.g., BAGE, GAGE-1, GAGE-2, MAGE-1, MAGE-3, N-acetylglucosaminyltransferase-V, p15), mutated antigens that are tumor-specific (β-catenin, MUM-1, CDK4), nonmelanoma antigens (e.g., HER-2/neu (breast and ovarian carcinoma), human papillomavirus-E6, E7 (cervical carcinoma), MUC-1 (breast, ovarian and pancreatic carcinoma)). For human tumor antigens recognized by T cells, see generally Robbins and Kawakami, 1996, Curr. Opin. Immunol. 8:628-36. Cancer vaccines may or may not be purified preparations.

In yet another embodiment, a BCA antagonist is used in association with a hormonal treatment. Hormonal therapeutic treatments comprise hormonal agonists, hormonal antagonists (e.g., flutamide, tamoxifen, leuprolide acetate (LUPRON), LH-RH antagonists), inhibitors of hormone biosynthesis and processing, and steroids (e.g., dexamethasone, retinoids, betamethasone, cortisol, cortisone, prednisone, dehydrotestosterone, glucocorticoids, mineralocorticoids, estrogen, testosterone, progestins), antigestagens (e.g., mifepristone, onapristone), and antiandrogens (e.g., cyproterone acetate).

In yet another embodiment, a BCA antagonist is used in association with a gene therapy program in the treatment of cancer. In one embodiment, gene therapy with recombinant cells secreting interleukin-2 is administered in combination with a BCA antagonist to prevent or treat cancer, particularly breast cancer (See, e.g., Deshmukh et al., 2001, J Neurosurg. 94:287-92).

In one embodiment, a BCA antagonist is administered, in combination with at least one cancer therapeutic agent, for a short treatment cycle to a cancer patient to treat cancer. The duration of treatment with the cancer therapeutic agent may vary according to the particular cancer therapeutic agent used. The invention also contemplates discontinuous administration or daily doses divided into several partial administrations. An appropriate treatment time for a particular cancer therapeutic agent will be appreciated by the skilled artisan, and the invention contemplates the continued assessment of optimal treatment schedules for each cancer therapeutic agent.

The present invention contemplates at least one cycle, preferably more than one cycle during which a single therapeutic or sequence of therapeutics is administered. An appropriate period of time for one cycle will be appreciated by the skilled artisan, as will the total number of cycles, and the interval between cycles. The invention contemplates the continued assessment of optimal treatment schedules for each BCA antagonist and cancer therapeutic agent.

5.6.19. Pharmaceutical Compositions.

Since inhibition of expression of a BCA gene or inhibition of a BCA protein can have significant therapeutic responses in a patient with a BCA-related disorder, the invention provides useful pharmaceutical compositions, treatment courses, and modes of delivery. Accordingly, in one embodiment, a pharmaceutical composition comprises a polynucleotide or polypeptide of the invention, and variants thereof, which refers to any pharmaceutically acceptable homologue, analogue, or fragment corresponding to the pharmaceutical composition of the invention. In another embodiment, the present invention provides for a pharmaceutical composition that comprises a BCA antagonist and a pharmaceutically acceptable carrier.

The phrase “pharmaceutically acceptable” refers to an agent that does not interfere with the effectiveness of the biological activity of an active ingredient, and which may be approved by a regulatory agency of the Federal government or a state government, or is listed in the U.S. Pharmacopeia or other generally recognized pharmacopeia for use in animals, more particularly for use in humans.

The carrier can be a diluent, adjuvant, excipient, or vehicle with which the therapeutic is administered. Such carriers can be sterile liquids, such as saline solutions in water, or oils, including those of petroleum, animal, vegetable or synthetic origin, such as peanut oil, soybean oil, mineral oil, sesame oil and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The carrier, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Examples of suitable pharmaceutical carriers are described in Remington's Pharmaceutical Sciences by E. W. Martin. Examples of suitable pharmaceutical carriers are a variety of cationic lipids, including, but not limited to N-(1(2,3-dioleyloxy)propyl)-N,N,N-trimethylammonium chloride (DOTMA) and diolesylphosphotidylethanolamine (DOPE). Liposomes are also suitable carriers for the antisense oligonucleotides of the invention. Such compositions should comprise a therapeutically effective amount of the compound, together with a suitable amount of carrier so as to provide the form for proper administration to the patient. The formulation should suit the mode of administration.

Pharmaceutically acceptable salts are prepared from pharmaceutically acceptable, essentially nontoxic, acids and bases, including inorganic and organic acids and bases. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, saline solutions, glycerol solutions, ethanol, N-(1(2,3-dioleyloxy)propyl)-N,N,N-trimethylammonium chloride (DOTMA), diolesylphosphotidylethanolamine (DOPE), and liposomes. Such compositions should comprise a therapeutically effective amount of the compound, together with a suitable amount of carrier so as to provide an appropriate formulation for administration to a patient. For example, oral administration requires enteric coatings to protect the antagonist from degradation within the gastrointestinal tract. In another example, the antagonist may be administered in a liposomal formulation to facilitate transport in circulatory system, effect delivery across cell membranes to intracellular sites, and shield the antagonist from degradative enzymes.

In another embodiment, a pharmaceutical composition comprises a BCA antagonist and one or more therapeutic agents and a pharmaceutically acceptable carrier. In a particular embodiment, the pharmaceutical composition comprises a BCA antagonist and one or more cancer therapeutic agents and a pharmaceutically acceptable carrier.

In a further embodiment, a pharmaceutical composition, comprising a BCA antagonist, with or without other therapeutic agents, and a pharmaceutically acceptable carrier, is at an effective dose.

The compositions of the invention can be formulated as neutral or salt forms. Pharmaceutically acceptable salts include those formed with free amino groups, such as for example, those derived from hydrochloric, phosphoric, acetic, oxalic, and tartaric acids, and those formed with free carboxyl groups, such as for example, those derived from sodium, potassium, ammonium, calcium, ferric hydroxides, isopropylamine, triethylamine, 2-ethylamino ethanol, histidine, and procaine.

In a preferred embodiment, the composition is formulated in accordance with routine procedures as a pharmaceutical composition adapted for subcutaneous injection or intravenous administration to humans. Typically, compositions for subcutaneous injection or intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water-free concentrate in a hermetically sealed container such as an ampule or sachette indicating the quantity of active agent. Where the composition is to be administered by infusion, it can be dispensed with an infusion bottle, bag, or other acceptable container, containing sterile pharmaceutical grade water, saline, or other acceptable diluents. Where the composition is administered by injection, an ampule of sterile water for injection or saline can be provided so that the ingredients may be mixed prior to administration.

The polynucleotides, polypeptides, and antibodies of the invention can be incorporated into pharmaceutical compositions suitable for administration. Such compositions typically comprise the polynucleotide, protein, or antibody, and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the pharmaceutical compositions of the invention is contemplated.

The invention includes methods for preparing pharmaceutical compositions for modulating the expression or activity of a polypeptide or nucleic acid of the invention. Such methods comprise formulating a pharmaceutically acceptable carrier with an agent which modulates expression or activity of a polypeptide or nucleic acid of the invention. Such compositions can further include additional active agents. Thus, the invention further includes methods for preparing a pharmaceutical composition by formulating a pharmaceutically acceptable carrier with an agent which modulates expression or activity of a polypeptide or nucleic acid of the invention and additional polynucleotides, polypeptides, and antibodies of the invention.

Selection of a preferred effective dose can be determined by a skilled artisan based upon the consideration of factors which will be known to one of ordinary skill in the art. Such factors include the particular form of a BCA antagonist and its pharmacokinetic parameters such as bioavailability, metabolism and half-life, which is established during the development procedures typically employed in obtaining regulatory approval of a pharmaceutical compound. Further factors that can be used to determine an effective dose include the disease to be treated, the benefit to be achieved in a patient, the patient's body mass, the patient's immune status, the route of administration, whether administration of a BCA antagonist and/or combination therapeutic agent is acute or chronic, concomitant medications, and other factors known by the skilled artisan to affect the efficacy of administered pharmaceutical agents.

In one embodiment, the pharmaceutical composition comprises a BCA antisense oligonucleotide at a dose of about 0.01 to 0.1, 0.1 to 1, 1 to 5, or 6 to 10 mg/kg/day; and a pharmaceutically acceptable carrier. The actual amount of any particular antisense oligonucleotide administered can depend on several factors, such as the type of disease, the toxicity of the antisense oligonucleotide to normal cells of the body, the rate of uptake of the antisense oligonucleotide by tumor cells, and the weight and age of the individual to whom the antisense oligonucleotide is administered. The skilled artisan will appreciate the factors that may interfere with the action or biological activity of the antisense oligonucleotide in vivo, an effective amount of the antisense oligonucleotide can be determined empirically by routine procedures, including, for example, via clinical trials.

In another embodiment, the pharmaceutical compositions of the invention comprise a BCA antisense oligonucleotide at a particularly high dose, which ranges from about 10 to 50 mg/kg/day. In a specific embodiment a particularly high dose of BCA antisense oligonucleotide, ranging from 11 to 15, 16 to 20, 21 to 25, 26 to 30, 31 to 35, 36 to 40, 41 to 45, or 46 to 50 mg/kg/day, is administered during a treatment cycle.

A preferred effective dose of a BCA antisense oligonucleotide can be determined by a skilled artisan, especially given that several antisense oligonucleotide compounds are currently undergoing clinical trials. These routine trials can establish the particular form of antisense oligonucleotide to be administered, an appropriate delivery route, and a particular antisense oligonucleotide's pharmacokinetic parameters such as bioavailability, metabolism, and half-life. Other factors typically considered during the course of a clinical trial are the patient's body mass, the patient's immune status, the disease to be treated, the benefit to be achieved in a patient, the route of administration, whether administration of an antisense oligonucleotide and/or combination therapeutic agent is acute or chronic, concomitant medications, and other factors known by the skilled artisan to affect the efficacy of administered pharmaceutical agents.

5.6.20. Modes of Administration.

Administration of the pharmaceutical compositions of the invention includes, but is not limited to, oral, intravenous infusion, subcutaneous injection, intramuscular, topical, depo injection, implantation, time-release mode, intracavitary, intranasal, inhalation, intratumor, intraocular, and controlled release. A pharmaceutical composition of the invention is formulated to be compatible with its intended route of administration. Examples of routes of administration include parenteral, e.g., intravenous, intramuscular, intraperitoneal, intraorbital, intracapsular, intraspinal, intrasternal, intra-arterial, intradermal, subcutaneous, oral (e.g., inhalation), transdermal (topical), transmucosal, and rectal administration. The skilled artisan can appreciate the specific advantages and disadvantages to be considered in choosing a mode of administration.

Multiple modes of administration are encompassed by the invention. For example, a BCA antagonist is administered by subcutaneous injection, whereas a combination therapeutic agent is administered by intravenous infusion.

A BCA antagonist can be administered before, during, and/or after the administration of one or more therapeutic agents. In one embodiment, a BCA antagonist can first be administered to cancer patient to reduce the expression of BCA, which increases the tumor's sensitivity to subsequent challenge with a cancer therapeutic agent. In another embodiment, a BCA antagonist can be administered after administration of a cancer therapeutic agent to reduce tumor expression of BCA, which can deter tumor resistance, and thereby prevent relapse or minimization of response to the cancer therapeutic agent. In yet another embodiment, there can be a period of overlap between the administration of BCA antagonist and one or more cancer therapeutic agents.

Moreover, administration of one or more species of BCA antagonist, with or without other therapeutic agents, may occur simultaneously (i.e., co-administration) or sequentially. In one embodiment, a BCA antagonist is first administered to increase sensitivity of a tumor to subsequent administration of a cancer therapeutic agent or irradiation therapy. In another embodiment, the periods of administration of one or more species of a BCA antagonist, with or without other therapeutic agents may overlap. For example, a BCA antagonist is administered for 14 days, and a second therapeutic agent is introduced beginning on the seventh day of BCA antagonist treatment, and treatment with the second therapeutic agent continues beyond the 14-day BCA antagonist treatment.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide, for example. The parenteral preparation can be enclosed in ampules, disposable syringes or multiple dose vials made of glass or plastic.

Pharmaceutical compositions adapted for parenteral administration include, but are not limited to, aqueous and non-aqueous sterile injectable solutions or suspensions, which may contain antioxidants, buffers, bacteriostats and solutes that render the compositions substantially isotonic with the blood of an intended recipient. Such compositions may also comprise water, alcohols, polyols, glycerine and vegetable oils, for example. Compositions adapted for parenteral administration can be packaged in unit-dose or multi-dose containers (e.g., sealed ampules and vials). These compositions can be stored in a freeze-dried (lyophilized) condition, which requires the addition of a sterile liquid carrier, e.g., sterile saline solution for injections, prior to use. Extemporaneous injection solutions and suspensions may be prepared from sterile powders, granules and tablets. Such compositions should comprise a therapeutically effective amount of a BCA antagonist and/or other therapeutic agent, together with a suitable amount of carrier so as to provide the form for proper administration to the patient. The formulation should suit the mode of administration.

Systemic administration can also be by transmucosal or transdermal means. For transmucosal or transdermal administration, penetrants appropriate to the barrier to be permeated are used in the formulation. Penetrants for transmucosal administration are generally known in the art, and include, for example, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

Pharmaceutical compositions adapted for transdermal administration can be provided as discrete patches intended to remain in intimate contact with the epidermis for a prolonged period of time. Pharmaceutical compositions adapted for topical administration may be provided as, for example, ointments, creams, suspensions, lotions, powders, solutions, pastes, gels, sprays, aerosols, or oils. A topical ointment or cream is preferably used for topical administration to the skin, mouth, eye or other external tissues. When formulated in an ointment, the active ingredient may be employed with either a paraffinic or a water-miscible ointment base. Alternatively, the active ingredient may be formulated in a cream with an oil-in-water base or a water-in-oil base.

Pharmaceutical compositions adapted for topical administration to the eye include, for example, eye drops or injectable compositions. In these compositions, the active ingredient can be dissolved or suspended in a suitable carrier, which includes, for example, an aqueous solvent with or without carboxymethylcellulose. Pharmaceutical compositions adapted for topical administration in the mouth include, for example, lozenges, pastilles and mouthwashes.

Pharmaceutical compositions adapted for oral administration may be provided, for example, as capsules, tablets, powders, granules, solutions, syrups, suspensions (in aqueous or non-aqueous liquids), edible foams, whips, or emulsions. Tablets or hard gelatine capsules may comprise, for example, lactose, starch or derivatives thereof, magnesium stearate, sodium saccharine, cellulose, magnesium carbonate, stearic acid or salts thereof. Soft gelatin capsules may comprise, for example, vegetable oils, waxes, fats, semi-solid, or liquid polyols. Solutions and syrups may comprise, for example, water, polyols and sugars.

Pharmaceutically compatible binding agents, and/or adjuvant materials can be included as part of the composition. The tablets, pills, capsules, and troches can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

An active agent intended for oral administration may be coated with or admixed with a material (e.g., glyceryl monostearate or glyceryl distearate) that delays disintegration or affects absorption of the active agent in the gastrointestinal tract. Thus, for example, the sustained release of an active agent may be achieved over many hours and, if necessary, the active agent can be protected from being degraded within the gastrointestinal tract. Taking advantage of the various pH and enzymatic conditions along the gastrointestinal tract, pharmaceutical compositions for oral administration may be formulated to facilitate release of an active agent at a particular gastrointestinal location. Oral formulations preferably comprise 10% to 95% active ingredient by weight.

Pharmaceutical compositions adapted for nasal administration can comprise solid carriers such as powders (preferably having a particle size in the range of 20 to 500 microns). Powders can be administered in the manner in which snuff is taken, i.e., by rapid inhalation through the nose from a container of powder held close to the nose. Alternatively, compositions adopted for nasal administration may comprise liquid carriers such as, for example, nasal sprays or nasal drops. These compositions may comprise aqueous or oil solutions of the active ingredient. Compositions for administration by inhalation may be supplied in specially adapted devices including, but not limited to, pressurized aerosols, nebulizers, or insufflators, which can be constructed so as to provide predetermined dosages of the active ingredient.

Pharmaceutical compositions adapted for rectal administration can be prepared in the form of suppositories (e.g., with conventional suppository bases such as cocoa butter and other glycerides) or retention enemas for rectal delivery. Pharmaceutical compositions adapted for vaginal administration may be provided, for example, as pessaries, tampons, creams, gels, pastes, foams, or spray formulations.

In one embodiment, a pharmaceutical composition of the invention is delivered by a controlled-release system. For example, the pharmaceutical composition may be administered using intravenous infusion, an implantable osmotic pump, a transdermal patch, liposomes, or other modes of administration. In one embodiment, a pump may he used (See, e.g., Langer, 1990, Science 249:1527-33; Sefton, 1987, CRC Crit. Ref. Biomed. Eng. 14:201; Buchwald et al., 1980, Surgery 88:507; Saudek et al., 1989, N. Encl. J. Med. 321:574). In another embodiment, the compound can be delivered in a vesicle, in particular a liposome (See, e.g., Langer, 1990, Science 249:1527-1533; Treat et al., 1989, in Liposomes in the Therapy of Infectious Disease and Cancer, Lopez-Berestein and Fidler (eds.) Liss, New York, pp. 353-65; Lopez-Berestein, ibid., pp. 317-27; International Patent Publication No. WO 91/04014; U.S. Pat. No. 4,704,355). In another embodiment, polymeric materials can be used (See, e.g., Medical Applications of Controlled Release, Langer and Wise (eds.) CRC Press: Boca Raton, Fla., 1974; Controlled Drug Bioavailability, Drug Product Design and Performance, Smolen and Ball (eds.) Wiley: New York (1984); Ranger and Peppas, 1953, J. Macromol. Sci. Rev. Macromol. Chem. 23:61; Levy et al., 1985, Science. 228:190; During et al., 1989, Ann Neurol. 25:351; Howard et al., 1989, J. Neurosurg. 71:105).

In one embodiment, the active compounds, which comprise polynucleotides, polypeptides, or antibodies of the invention, are prepared with carriers that will protect the compound from rapid elimination from the body. Such carriers can be a controlled release formulation, which includes, but is not limited to, implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

In a particular embodiment, polypeptides of the invention can be administered using a biodegradable polymer having reverse thermal gelatin properties (See, e.g., U.S. Pat. No. 5,702,717).

In yet another embodiment, a controlled release system can be placed in proximity of the target. For example, a micropump may deliver controlled doses directly into the axillary lymph node region, thereby requiring only a fraction of the systemic dose (See, e.g., Goodson, 1984, in Medical Applications of Controlled Release, vol. 2, pp. 115-138).

In one embodiment, it may be desirable to administer a pharmaceutical composition of the invention locally to the area in need of treatment; this may be achieved, for example, by local infusion during surgery, topical application (e.g., in conjunction with a wound dressing after surgery), injection, by means of a catheter, by means of a suppository, or by means of an implant. An implant can be of a porous, non-porous, or gelatinous material, including membranes, such as sialastic membranes, or fibers. Suppositories generally comprise active ingredients in the range of 0.5% to 10% by weight.

Pharmaceutical compositions suitable for injectable use include sterile aqueous solutions, or dispersions, or sterile powders (for the extemporaneous preparation of sterile injectable solutions or dispersions). For intravenous administration, suitable carriers include physiological saline, bacteriostatic water, Cremophor EL™ (BASF; Parsippany, N.J.) or phosphate buffered saline (PBS). The carrier can be a solvent or dispersion medium comprising, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion, or by the use of a surfactant. Prevention of the action of microorganisms can be achieved by various antibacterial and antifungal agents, such as for example, parabens, chlorobutanol, phenol, ascorbic acid, and thimerosal. It can be preferable to include in the composition isotonic agents, such as for example, sugars, polyalcohols (e.g., mannitol), sorbitol, and sodium chloride. Prolonged absorption of the injectable compositions can be brought about by including in the composition an agent which delays absorption, such as for example, aluminum monostearate and gelatin.

Sterile injectable solutions can be prepared by incorporating the required amount of an active compound (e.g., a polypeptide or antibody) in an appropriate solvent with one or a combination of ingredients enumerated above, as required, followed by filtered sterilization. Generally, dispersions are prepared by incorporating the active compound into a sterile vehicle which comprises a basic dispersion medium and the required other ingredients from those enumerated above. In the case of sterile powders for the preparation of sterile injectable solutions, the preferred methods of preparation are vacuum drying and freeze-drying which yields a powder comprising the active ingredient.

For administration by inhalation, the compounds are delivered in the form of an aerosol spray from a pressurized container or dispenser which comprises a suitable propellant, e.g., a gas such as carbon dioxide, or a nebulizer.

Oral or parenteral compositions can be formulated in dosage unit form for ease of administration and uniformity of dosage. Dosage unit form as used herein refers to physically discrete units suited as unitary dosages for the subject to be treated, such that each unit contains a predetermined quantity of active compound, which is calculated to produce the desired therapeutic effect, and a pharmaceutical carrier. The skilled artisan will appreciate that dosage unit forms are dependent on the unique characteristics of the active compound, the particular therapeutic effect to be achieved, and the limitations inherent in the art of compounding such an active compound for human administration.

For antibodies, the preferred dosage is 0.1 mg/kg to 100 mg/kg of body weight (generally 10 mg/kg to 20 mg/kg). For example, if the antibody is to act in the brain, a dosage of 50 mg/kg to 100 mg/kg is usually appropriate. Since partially human antibodies and fully human antibodies generally have a longer half-life in a patient than other antibodies, lower dosages and less frequent administration is possible. Modifications, such as lipidation, can be used to stabilize antibodies and to enhance uptake and tissue penetration (See, e.g., Cruikshank et al., 1997, J Acquir Immune Defic Syndr Hum Retrovirol. 14(3):193-203).

In one embodiment, a therapeutically effective amount of a polypeptide of the invention ranges from about 0.001 to 30 mg/kg body weight. In another embodiment, a therapeutically effective amount of a polypeptide of the invention ranges from about 0.01 to 25 mg/kg body weight. In another embodiment, a therapeutically effective amount of a polypeptide of the invention ranges from about 0.1 to 20 mg/kg body weight. In yet another embodiment, a therapeutically effective amount of a polypeptide of the invention ranges from about 1 to 10 mg/kg, 2 to 9 mg/kg, 3 to 8 mg/kg, 4 to 7 mg/kg, or 5 to 6 mg/kg body weight.

The skilled artisan will appreciate that certain factors may influence the dose necessary to effectively treat a subject, which factors include, but are not limited to, previous treatment regimens, severity of the disease or disorder, general health and/or age of the subject, and concurrent diseases. Moreover, treatment of a subject with a therapeutically effective amount of a protein, polypeptide, or antibody can include a single treatment or, preferably, can include a series of treatments. In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

5.6.21. Kits.

The invention also encompasses kits for detecting the presence of a BCA polypeptide or polynucleotide of the invention in a biological sample (a test sample). Such kits can be used to determine if a subject is suffering from or is at increased risk of developing a disorder associated with aberrant expression of a polypeptide of the invention as discussed, for example, in sections above relating to uses of the sequences of the invention.

In an exemplary embodiment, a kit comprises, in a first container, a purified BCA nucleic acid, BCA polypeptide, BCA agonist, BCA antagonist, and in a second container, a molecule that binds to the BCA nucleic acid, BCA polypeptide, BCA agonist, BCA antagonist when bound to an analyte in a biological sample. The molecule can be, for example, a detectable tag that recognizes a complex comprising BCA and the analyte such that the interaction between BCA and the analyte is identified. Many detectable tags, such as antibodies, for such use are well known in the art and readily available.

For example, kits can be used to determine if a subject is suffering from or is at increased risk of disorders such as cancer, in particular hormone-sensitive cancers, such as but not limited to cancer of the breast, ovary, uterus, prostate, testis, skin and brain.

In another example, kits can be used to determine if a subject is suffering from or is at risk for a disorder associated with aberrant expression of a polypeptide of the invention.

The kit, for example, can comprise a labeled compound or agent capable of detecting the BCA polypeptide or BCA mRNA encoding the polypeptide in a biological sample and means for determining the amount of the polypeptide or mRNA in the sample (e.g., an antibody which binds the polypeptide or an oligonucleotide probe which binds to DNA or mRNA encoding the polypeptide). Kits can also include instructions for observing that the tested subject is suffering from or is at risk of developing a disorder associated with aberrant expression of the polypeptide if the amount of the polypeptide or mRNA encoding the polypeptide is above or below a normal level.

For antibody-based kits, the kit can comprise, for example: (1) a first antibody (e.g., attached to a solid support) which binds to a polypeptide of the invention; and, optionally, (2) a second, different antibody which binds to either the BCA polypeptide or the first antibody and is conjugated to a detectable agent.

For oligonucleotide-based kits, the kit can comprise, for example: (1) an oligonucleotide, e.g., a detectably labeled oligonucleotide, which hybridizes to a BCA polynucleotide encoding a polypeptide of the invention or (2) a pair of primers useful for amplifying a polynucleotide encoding a polypeptide of the invention. The kit can also comprise, e.g., a buffering agent, a preservative, or a protein stabilizing agent. The kit can also comprise components necessary for detecting the detectable agent (e.g., an enzyme or a substrate). The kit can also comprise a control sample or a series of control samples which can be assayed and compared to the test sample contained. Each component of the kit is usually enclosed within an individual container and all of the various containers are within a single package along with instructions for observing whether the tested subject is suffering from or is at risk of developing a disorder associated with aberrant expression of the polypeptide.

The invention provides a kit containing an antibody of the invention conjugated to a detectable substance, and instructions for use. Still another aspect of the invention is a pharmaceutical composition comprising an antibody of the invention and a pharmaceutically acceptable carrier. In preferred embodiments, the pharmaceutical composition comprises an antibody of the invention, a therapeutic moiety, and a pharmaceutically acceptable carrier.

The pharmaceutical compositions of the invention can be included in a container, pack, or dispenser together with instructions for administration.

The present invention may be better understood by reference to the following non-limiting examples, which are provided only as exemplary of the invention. The following examples are presented to more fully illustrate the preferred embodiments of the invention. The examples should in no way be construed, however, as limiting the broader scope of the invention.

### 6. EXAMPLES

### 6.1. Identification of BCA Genes.

In order to precisely define genes that are associated with the initiation and development of breast cancer, more than 1000 cDNA clones for genes that may be activated or inactivated during progression of breast cancer were isolated. The isolation of such clones was accomplished by subtractive hybridization and differential display methods using matched breast tumor and normal breast cell line RNAs.

RNA was prepared from breast cell lines derived from a tumor and the adjacent normal tissue of the same patient. cDNAs from the normal breast cell line, Hs578Bst, were subtracted from cDNAs of the breast carcinoma cell line Hs578T.

All cDNA sequences from the breast cancer subtractive cloning library were compared by BLAST to the entire non-redundant GenBank database. This search was further refined by comparison in different subsets of GenBank, for example, the vector database and the E. Coli genome.

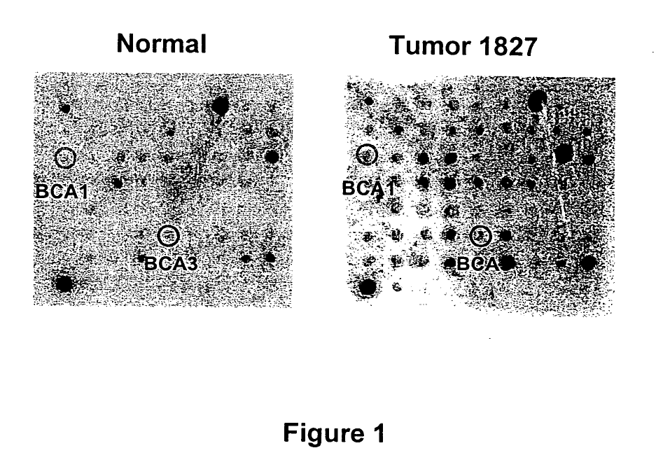

Seven genes, namely BCA1-7, were identified. Two genes, BCA1 and BCA3 are expressed more highly in a breast tumor tissue than in normal breast tissue (FIG. 1).

In order to characterize complete genes and gather indications of the function of their expressed proteins in breast cancer, each novel sequence was translated using web-based tools available at the Baylor College of Medicine Search Launcher home page. It was possible that some known protein sequences were not recognized by DNA searches, and thus each ORF was compared to protein databases (EMBL, ExPASY), as well as to the translated non-redundant GenBank database using the tBlastn program. As the subtractive cloning procedure was designed to capture sequences close to the 3′ end of expressed genes with the oligo-dT primer that was used, some of the translated sequences were short. Although cloned cDNA fragment sizes in the breast cancer subtractive, library are approximately 500 bp on average, initial “tag” sequencing resulted in between 100 and 300 bp sequences for most clones. Full-length clones were obtained using bioinformatic databases.

Such analysis demonstrated that BCA3 has at least three different mRNAs. After examination of corresponding genomic sequences, it was predicted that the three mRNAs arise from alternative splicing, and has been confirmed by RT-PCR. Knowing where the first exon is within a genomic clone allows one to survey the 5′ flanking sequence for cis-acting regulatory elements, providing clues as to which transcription factors may affect each breast cancer specific gene.

The putative ORFs for consensus sequences defined by Protium and other computed databases were searched to gather as much existing information as to the in vivo roles of proteins encoded by the breast cancer library. An access point is the SMART interface, which combines multiple protein sequence databases for analysis of each query sequence, and has excellent links to multiple sources for each group of proteins from which consensus residues were identified. By such analyses, the existence of a RING-H2 domain in BCA1 and BCA2, and a WW-domain binding PPPPY motif in BCA1 has been established.

### 6.2. Chromosome-Mapping of the BCA1-7 Genes.

The chromosomal localization of seven cDNAs that are differentially expressed in breast carcinoma cell lines were determined. Their chromosomal localization to regions frequently altered in breast and other cancers is consistent with a role in the development and progression of breast cancer.

The chromosomal localization of each clone was performed by fluorescence in situ hybridization (FISH). The seven cDNAs (260-280 bp) were initially screened on the RPCI-1 human P1-derived artificial chromosome (PAC) library (http://bacpac.med.buffalo.edu) to obtain corresponding genomic PAC clones. Several of the cDNAs identified multiple PACs, thus PCR analysis was performed using primers (Table 1) from the original cDNAs to confirm genomic clone identity for each PAC. PACs containing PCR-positive inserts for each cDNA (Table 1) were then mapped by FISH analysis to normal human metaphase chromosomes. Subchromosomal banding localization of three PAC clones is shown in FIG. 2. The FISH mapping data was confirmed by screening of a NIGMS monochromosomal rodent/human somatic cell hybrid panel #2, as well as by radiation hybrid and YAC contig mapping (Table 1).

Sequence analysis of the seven cDNA clones revealed that only one clone, BCA7, demonstrated an ORF that was identical to a known human gene, human Cyclin I (Nakamura et al., 1995, “Cyclin I: a new cyclin encoded by a gene isolated from human brain”, Experimental Cell Research 221:534-542). As shown in Table 1, the remaining six cDNAs (BCA1, BCA2, BCA3, BCA4, BCA5, and BCA6) identified ESTs but had no matches to any functionally characterized human gene in public databases (Table 1).

Two clones, BCA1 and BCA2, showed a weak similarity to the Drosophila Goliath (g1) protein, a zinc finger protein with a putative transcription factor function regulating gene expression during mesoderm formation (Bouchard et al., 1993, “The Drosophila melanogaster developmental gene g1 encodes a variant zinc-finger-motif protein”, Gene 125: 205-209).

BCA5, demonstrated a high similarity (p=3.5e-299) to an SEC61 gamma subunit that is involved in protein transport of the rough endoplasmic and is found in dog (Canis familiarus) and mouse (Mus musculus)(Gordon et al., 1992, “Tumor necrosis factor induces genes involved in inflammation, cellular and tissue repair, and metabolism in murine fibroblasts”, Journal of Immunology 148:4021-4027).

The BCA4 sequence was weakly similar to an ORF in yeast and the remaining two novel cDNAs, BCA3 and BCA6, showed no similarity to any known functional genes, though did identify ESTs.

The BCA7 clone mapped to 4q21, one of a few regions shown by comparative genomic hybridization (“CGH”) to be amplified in primary breast carcinoma (Tirkkonen et al., 1998, “Molecular cytogenetics of primary breast cancer by CGH”, Genes, Chromosomes & Cancer 21:177-184). This finding supports a role for the BCA7 gene in the more aggressive form of breast carcinoma.

Two of the novel clones mapped to regions of chromosome 1 (FIG. 2), that have been implicated in breast and other cancers (Bieche et al., 1995, “Loss and gain of distinct regions of chromosome 1q in primary breast cancer”, Clinical Cancer Research 1:123-127; Brenner et al., 1997, “The genetics of sporadic breast cancer”, Progress in Clinical & Biological Research 396:63-82; Tirkkonen et al., 1998, “Molecular cytogenetics of primary breast cancer by CGH”, Genes, Chromosomes & Cancer 21:177-184). One of these clones, BCA1 (1p32), localized to a region that coincides with one of four minimally deleted regions of 1p found in breast cancer (Hoggard et al., 1995, “Allelic imbalance on chromosome 1 in human breast cancer. II. Microsatellite repeat analysis”, Genes, Chromosomes & Cancer 12: 24-31). Allelic loss of the 1p32 region has also been noted in other malignancies, such as neuroblastoma, in which loss of the 1p32-pter region associated with poor prognosis (Takeda et al., 1994, “There may be two tumor suppressor genes on chromosome arm Ip closely associated with biologically distinct subtypes of neuroblastoma”, Genes, Chromosomes & Cancer 10: 30-39).

BCA2 localized to 1q21 (FIG. 2), identified as a smallest commonly deleted region within the larger 1q21-23 locus which has shown a high frequency of allelic loss in primary breast carcinoma (Bieche et al., 1995, “Loss and gain of distinct regions of chromosome 1q in primary breast cancer”, Clinical Cancer Research 1:123-127). Additionally, an increase in 1q copy number is also a common finding in breast carcinoma by CGH and has been demonstrated in 50% of early stage node negative breast carcinomas (Isola et al., 1995, “Genetic aberrations detected by comparative genomic hybridization predict outcome in node-negative breast cancer”, American Journal of Pathology 147: 905-911) and 67% of primary breast carcinomas (Tirkkonen et al., 1998, “Molecular cytogenetics of primary breast cancer by CGH”, Genes, Chromosomes & Cancer 21:177-184). The finding of both allelic loss and gain of the 1q21 region indicates the presence of both tumor suppressor genes and oncogenes in this region, and highlights the merits of detailed analysis of this locus.

The BCA3 clone mapped to 6q14-15 (FIG. 2), another chromosomal region in which allelic loss in breast cancer has been reported by both LOH (loss of heterozygousity) and CGH (comparison of genomic hybridization) studies (Sheng et al., 1996, “Multiple regions of chromosome 6q affected by loss of heterozygosity in primary human breast carcinomas”, British Journal of Cancer 73:144-147; Nishizaki et al., 1997, “Genetic alterations in primary breast cancers and their metastases: direct comparison using modified comparative genomic hybridization”, Genes, Chromosomes & Cancer 19:267-272). Similar loss of 6q has been reported in other tumors such as lung (Merlo et al., 1994, “Homozygous deletion on chromosome 9p and loss of heterozygosity on 9q, 6p, and 6q in primary human small cell lung cancer”, Cancer Research 54:2322-2326) and prostate carcinoma (Hyytinen et al., 1997, “Genetic changes associated with the acquisition of androgen-independent growth, tumorigenicity and metastatic potential in a prostate cancer model”, British Journal of Cancer 75: 190-195).

The remaining three novel cDNA clones (i.e., BCA4, BCA5 and BCA6) mapped to chromosomal regions not currently associated with development of primary breast cancer. However, these loci (i.e., 5q33, 7p12 and 2p11-12, respectively) have demonstrated aberrations in some tumor types or contain genes associated with regulation of cell growth (Veronese et al., 1995, “Detection of myc translocations in lymphoma cells by fluorescence in situ hybridization with yeast artificial chromosomes”, Blood 85:2132-2138; Pedersen, 1996, “Anatomy of the 5q-deletion: different sex ratios and deleted 5q bands in MDS and AML”, Leukemia 10:1883-1890; Collins, 1993, “Amplified genes in human gliomas”, Seminars in Cancer Biology 4:27-32).

The only clone that identified a known functionally characterized human gene was BCA7, a member of the cyclin family of cell cycle regulatory proteins (Nakamura et al., 1995, “Cyclin I: a new cyclin encoded by a gene isolated from human brain”, Experimental Cell Research 221:534-542). The predicted amino acid sequence of BCA7 shared a number of similarities with other cyclins including the cyclin box that encodes the region responsible for binding to the respective cyclin dependent kinases (CDKs). Overexpression of cyclins such as Cyclin D1 and D3 leads to loss of normal growth control and is thought to play an important role in tumor development and progression. Most members of the cyclin family are integrally involved in regulation of cell cycle, and are highly expressed during interphase and destroyed at the end of each cell cycle (Glotzer et al., 1991, “Cyclin is degraded by the ubiquitin pathway”, Nature 349:132-138).

Expression of Cyclin I isolated from brain, however, was found to be highest in the post mitotic tissues of brain, heart and skeletal muscle and independent of the cell cycle (Nakamura et al., 1995, “Cyclin I: a new cyclin encoded by a gene isolated from human brain”, Experimental Cell Research 221:534-542). Furthermore, identification of a novel isoform of Cyclin I (Cyclin ITI) in testis (Zhu et al., 1998, “Expression of a novel isoform of cyclin I in human testis”, Biochemical & Biophysical Research Communications 249:56-60) suggests there may be other tissue specific isoforms of this cyclin which may demonstrate tissue-specific functions.

### 6.3. Differential Expression of BCA Genes in Breast Cancer Cells, Tissues and Cell Lines

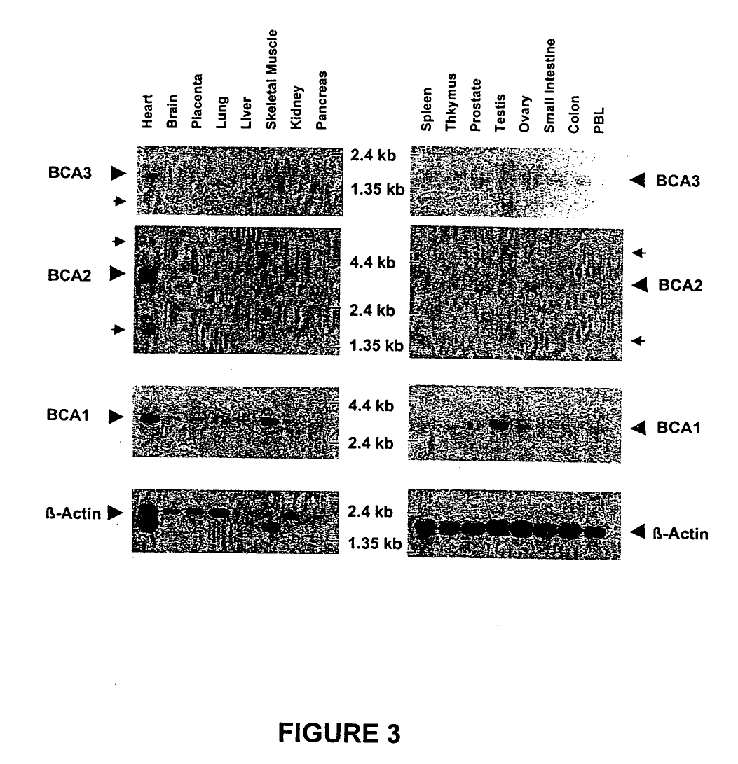

Northern blot analysis was performed to examine the differential expression of BCA1, BCA2 or BCA3 in tissue (FIG. 3). RNA from various human tissues or breast cancer cell lines, including a cell line that does not express estrogen receptors, were subjected to gel electrophoresis and transferred to a membrane. The transferred RNA was hybridized with radiolabeled cDNA probes.

BCA2 was most abundant in heart, skeletal muscle and testis. The mRNA was also present in placenta, lung, liver, kidney, pancreas, thymus, prostrate, ovary, small intestine and colon. Three bands were present in each tissue. The most predominant was 3.4-kb message, while a 4.5 kb and a 1.4 kb message were also present to a lesser extent (FIG. 2B). Prostate and breast tumor cell lines expressed BCA2.

BCA1 was present as a single message of 3.4 kb. BCA1 was abundant in the testis, though significant expression was detected in prostate, ovaries and heart, brain, placenta, skeletal muscle, kidney, prostate and ovary. Moderate levels of expression were also noted in lung, liver, pancreas, small intestine, colon and PBL. (FIG. 3). Prostate and breast tumor cell lines expressed BCA1.

BCA3 was predominately expressed in heart. It was also present at low levels in testis, ovary, and skeletal muscle. No expression was detected in prostate. BCA3 is expressed in breast cancer cell lines, but is found at very low levels in normal breast tissue.

Moreover, BCA3 is found at high levels in prostate tumor cell lines, LNCAP and DU-145 (FIG. 3).

Two of the genes were analyzed in estrogen-receptor-negative (“ER−ve”) breast carcinoma cells and in estrogen-receptor-positive (“ER+ve”) breast cell lines. BCA7 was expressed at higher levels in estrogen receptor ER−ve breast carcinoma cell lines as compared to its expression in ER+ve cell lines (FIG. 4B), indicating a role in the increased aggressiveness of these tumors. BCA4 expression however, did not correlate with the presence or absence of estrogen receptors, and demonstrated highest mRNA expression in lane 3 (MDA-MB-231, ER−ve) and lane 5 (MCF-7, ER+ve). Notably, BCA1 and BCA5 showed highest expression in the ER−ve cells HTB-126 and MDA-MS-468 (lanes 4 and 6) whereas BCA2 demonstrated high expression in the ER+ve MCF-7 cells indicating different roles of these genes in breast tumor progression (FIG. 4B).

Elevated expression of BCA7 in the ER−ve breast cancer cell lines (FIG. 4B) indicates a role for this gene in the more aggressive form of breast carcinoma. Thus, BCA7 can be useful for establishing a prognosis for, arid staging, breast cancer.

### 6.4. Preparation of Antibodies to Breast-Cancer-Associated Genes

In order to study the protein expression patterns of the breast-cancer-associated genes, peptides were synthesized and used as immunogens to generate antibodies. Examples of peptides useful for generating antibodies are listed below:

Peptide Immunogens

| | | |
|---|---|---|
| cDNA | Peptide | Amino Acid Sequence |

| | | |
|---|---|---|
| BCA1 | C14N | C-M-E-P-V-D-A-A-L-L-S-S-Y-E-T-N |
| BCA2 | N13F | N-R-S-N-D-S-Q-L-N-D-R-W-T-F |
| BCA3 | A14V | A-V-D-S-G-Q-S-V-D-L-V-F-P-V |

### 6.5. BCA1

6.5.1 BCA1 Nucleic Acid Sequence.

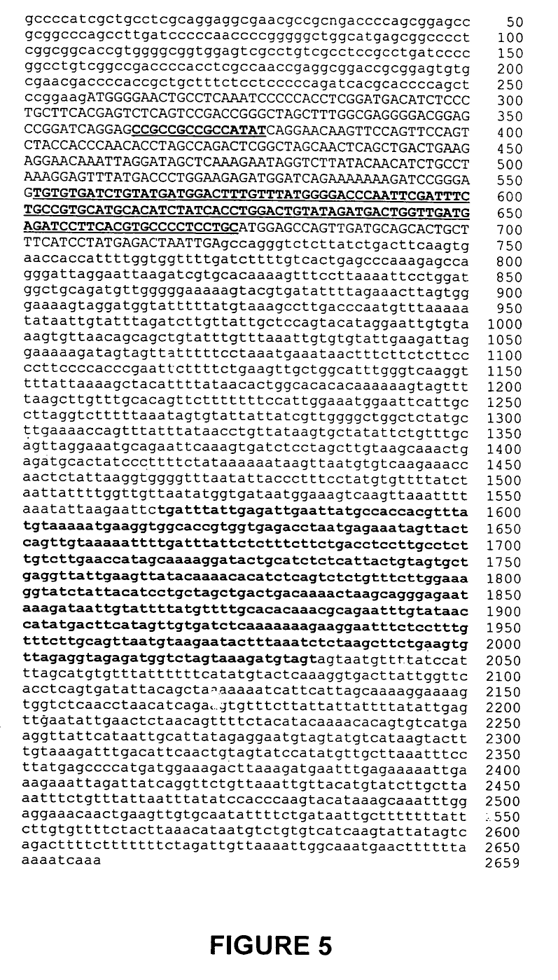

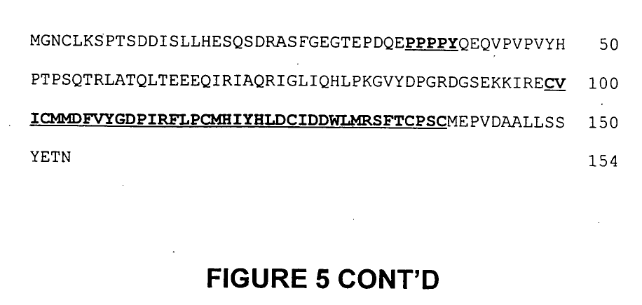

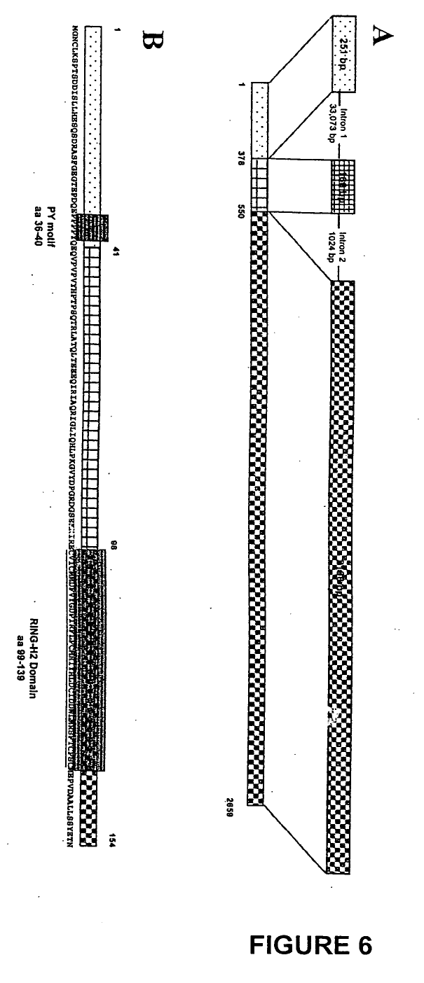

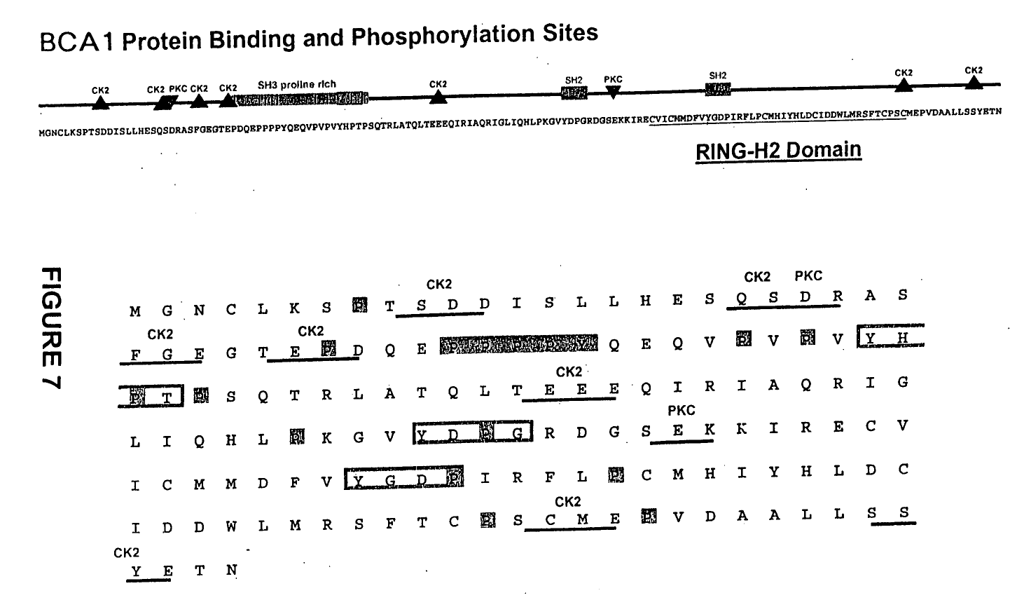

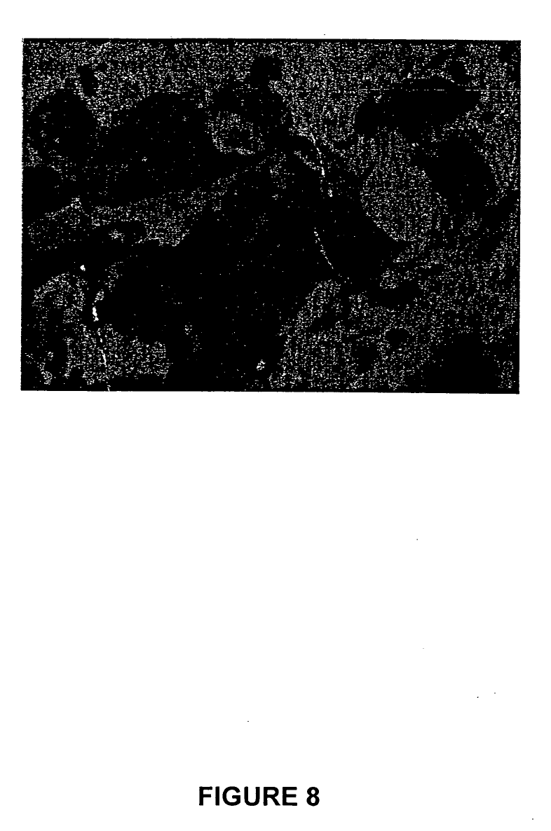

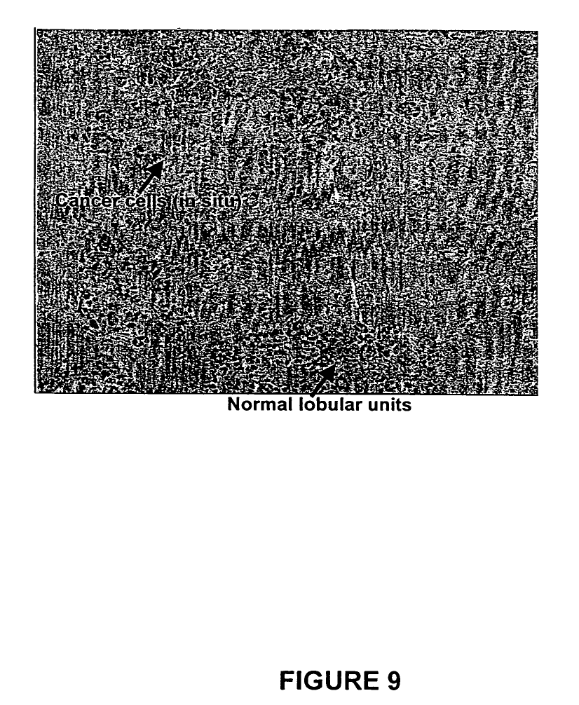

The BCA1 gene is shown in FIG. 5. The original clone was extended with overlapping Genbank EST sequences. Additional searches revealed that a similar cDNA, RNF11, had been identified previously (Genbank Accession AB024703). Extension of RNF11 added 124 nucleotides to the 5′ end and 143 nucleotides to the 3′ end. The entire cDNA sequence is 2659 bases and is located in Human Genome Project segment NT_{—}026948. The gene is organized into three exons. Exon I (251 bp) and exon II (166 bp) are separated by 33,073 bases. Exon II and exon III (2106 bp) are separated by 1024 bases. Exon I has a PPPPY (SEQ ID NO.: 16) motif at the carboxy terminus. Exon II contains two consensus Y-x-x-hydrophobic motifs, indicative of potential SH2 domain binding sites (Pawson, 1997). Exon III is characterized by a RING-H2 domain. One splice junction occurs immediately after a WW-domain-binding motif and another splice junction is located immediately before the RING-H2 domain. The BCA1 open-reading frame begins within exon I and ends near the 5′ end of exon III. The predicted protein sequence of BCA1 features several phosphorylation sites, including at least seven casein kinase-2 (“CK2”) and two protein kinase C (“PKC”) phosphorylation sites (FIG. 7).

The BCA1 polynucleotide has two SH2 (src homology-2) domains. SH2 domains, which are approximately 100 amino acid residues in length, are involved in transmission of ligand-induced signals to cell components via recognition of phosphorylated tyrosines on the receptor protein-tyrosine kinase by cytoplasmic SH2-containing signal transduction proteins. SH2 domains recognize phosphorylated tyrosines with specificity, and complement signaling by catalytic kinase activity by communicating the phosphorylation states of signal transduction proteins to elements of the signaling pathway. SH2 domains and their role in determining the specificity of protein-protein interactions are reviewed by Pawson and Schlesinger (1993, Current Biology 3:434-442).

SH2 domains are found in cytoplasmic non-receptor tyrosine kinases as well as in a number of other proteins that play key roles in signal transduction. Signal transduction proteins that are not tyrosine kinases but which contain SH2 domains include, for example, src (and the src family kinases such as Lck), Ab1, phospholipase C-γ1 (Cantley et al., 1991, Cell 64:281-302), the p85 subunit of phosphatidylinositol-3-OH kinase (PIK) (Skolnik, 1991, Cell 65:83-90), ras guanine triphosphatase-activating protein (GAP) (Vogel et al., 1988, “Cloning of bovine GAP and its interaction with oncogenic ras p21”, Nature 335:90-93; Lowenstein et al., 1992, Cell 70:431-442), insulin receptor substrate-1 (IRS-1) (White, 1994, “The IRS-1 Signalling System”, Current Opinion in Genetics & Development 4:47-54), protein-tyrosine phosphatases (Matthews et al., 1992, Mol. Cell Biol. 12:2396-2405), Feng and Pawson, 1994, Trends in Genetics 10:54-58), grb-2, shc (Pelicci, 1992, Cell 70:93-104), ZAP-70 (Chan et al., 1992, Cell 71:649-662), syk (Taniguchi et al., 1991, J. Biol. Chem. 266:15790-15796), and the transcription factors of the Stat family (Darnell et al., 1994, Science 264:1415-1421);, Zhong et al, 1994, Proc. Natl. Acad. Sci. 91:4806-4810).

While normal activation of tyrosine kinases induces cells to, inter alia, proliferate, migrate or differentiate, aberrant tyrosine kinase activity (e.g., constitutive activation) can cause excessive cell growth or proliferation. Thus, the present invention provides a method for suppressing tyrosine kinase activity of a BCA polypeptide.

The present invention also provides a BCA polypeptide that comprises an SH2 domain for screening and identifying small molecule inhibitors capable of competing with or blocking the binding of phosphorylated targets to SH2 domains of the BCA polypeptides. Such small molecules can, for example, be useful for inhibiting cell proliferation in vitro and/or in vivo. in cells, thereby preventing cell proliferation. Techniques and methods for screening compounds that bind to SH2 domains can be adapted to screen compounds that bind SH2-domain containing BCA polypeptides of the invention (see, e.g., U.S. Pat. No. 5,858,686 which is incorporated herein by reference in its entirety).

Compounds that are found to bind SH2 domains of other proteins can also be screened to identify those compounds that bind to a BCA polypeptide of the invention (see, e.g., U.S. Pat. Nos. 5,710,129, 5,922,697, and 6,054,470, each of which is incorporated herein by reference in its entirety). Accordingly, the use of any such compounds known in the art to bind SH2 domains, and that bind to a BCA polypeptide, for treatment or prevention of any disease, including cancer, is encompassed by the present invention.

The crystal structure of the src SH2 domain in a complex with several phosphotyrosine-containing peptides has been determined (see, e.g., Waksman et al., 1992, Nature 358:646-653 and Waksman et al., 1993, Cell 72:779-790). The structural information can be used to create models of the SH2 domains of the BCA polypeptides for use in rational drug design.

Methods for modifying a SH2 domain of the BCA polypeptides are also encompassed by the present invention. Methods, such as those described in U.S. Pat. No. 5,786,454, can be used to generate modified SH2 domains.

Separate regions of BCA1 comprise consensus amino acid sequences for a WW-domain binding motif and a RING-H2 domain. The presence of these domains in BCA1 increases the likelihood that true protein partners will be identified in a yeast two-hybrid assay. The PPPPY sequence of BCA1 is identical to that of LMP2A, which has been shown to bind to WW-domain polypeptides derived from AIP4, an E3 ubiquitin ligase which mediates the ubiquitination and degradation of the breast tumor suppressor Syk. The RING-H2 domain in BCA1 (CxxCxxxxxxxxxxxxxxCxHxxHxxCxxxxxxxxxxCxxC) (SEQ ID NO.: 28) and its WW-domain binding motif (PPPPY) (SEQ ID NO.: 16) indicates a role in protein-protein interactions such as, for example, ubiquitin-dependent protein degradation.

The full length sequence of BCA1 was extended in silico using overlapping EST's. BCA1 encodes a human homologue of the hypothetical mouse RING-H2 protein, RNF11. RNF11 is expressed at high levels in breast and prostate cancer cells, and contains motifs known to interact with other proteins involved in oncogenesis.

Splice junctions occur immediately after the WW-binding motif and immediately before the RING-H2 domain.

6.5.2 Homologs of BCA1

A search for proteins homologous to that encoded by BCA1, identified homologues in mouse, African frog (Xenopus laevis), western frog, zebrafish, pufferfish, nematode, and fly. Exons 2 and 3 of BCA1 are present in the pufferfish genomic clone AF022814. The zebrafish homologue, which extends into exon 3, has been deduced from zebrafish ESTs BG738008 and BM035975. The African frog sequence was deduced from Xenopus laevis cDNA clone BI444408. The nematode genomic clone has five exons rather than the three that encode the human gene. However, the final exon of both genes begins with the first amino acid of the RING-H2 domain. Sequence identity between the human and nematode RING-H2 domains is 68%, however there is no PPPPY (SEQ ID NO.: 16) WW-domain in the nematode sequence. Similarly, the Drosophila sequence does not contain a PPPPY (SEQ ID NO.: 16) WW-domain. However, Drosophila BCA1 is 85% homologous to the human sequence if conservative substitutions are considered.

6.5.3 Antibodies to BCA1

Antibodies were raised in rabbits against a synthetic peptide sequence derived from the C-terminal region of BCA1 and conjugated to keyhole limpet hemocyanin (“KLH”). IgG polyclonal antibodies were purified on a peptide affinity column. The specificity of the BCA1 antibodies obtained was confirmed by recognition of a BCA1 fusion protein and western blot analysis.

6.5.4 Expression of BCA1

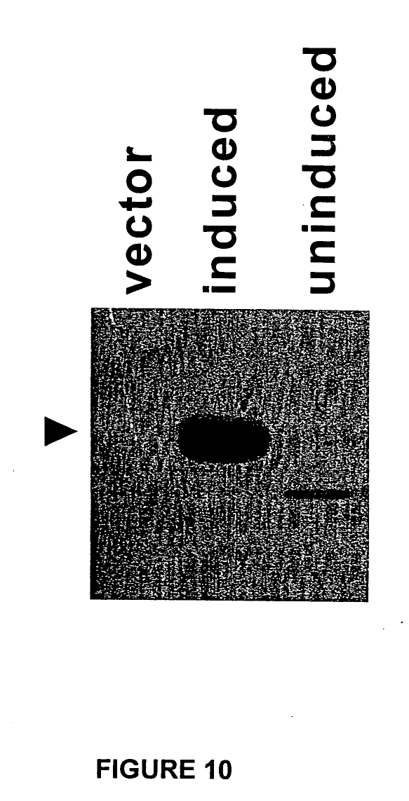

In order to express BCA1 in bacteria, PCR-generated BCA1 was subcloned into an IPTG-inducible vector, pGEX-5X-3 vector. Host bacteria were transformed with the pGEX-BCA1 vector. After induction with IPTG, a GST-BCA1 fusion protein was generated. Extracts of IPTG-induced cells bearing GST-vector or GST-BCA1, or uninduced cells containing GST-BCA1 were prepared and examined by western blot with the anti-BCA1 antibody (FIG. 4C, FIG. 10).

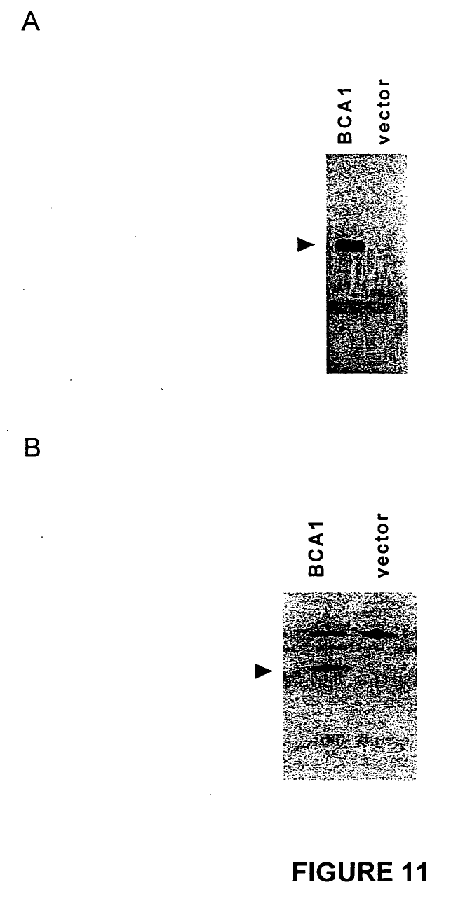

BCA1 has also been expressed in vitro in a rabbit reticulocyte lysate system. BCA1 was inserted into a pCMV vector which comprises a FLAG epitope. An autoradiogram of the ³⁵S-labeled protein and an immunoblot using anti-FLAG antibody are shown in FIG. 11. BCA1 expressed in vitro is visualized as a single band in the BCA1 lane. BCA1 is not visible in the vector alone lane.

6.5.5 Immunohistochemistry

Purified anti-BCA1 antibody was used to monitor protein expression in primary tumor and normal breast tissues. BCA1 is predominantly expressed in cytoplasm and is undetectable in nuclei. Staining of primary breast tissues revealed anti-BCA1 immunoreactivity in cancer cells, but not in surrounding stroma or in normal breast epithelial cells. Furthermore, in situ tumor cells surrounding and filling ductal tissue were strongly stained as compared to normal duct cells.

6.5.6 Phosphorylation

BCA1-tag and v-src were transiently co-transfected into 293-T cells. Whole cell lysates from the transfected cells were probed with anti-phosphotyrosine monoclonal antibody which did not detect any tyrosine-phosphorylated BCA1

6.5.7 Yeast Two-Hybrid Analysis

The yeast two-hybrid method was used to identify and isolate cDNAs that encode proteins which interact with BCA1. The screen was performed to distinguish between proteins that interact with the PPPPY (SEQ ID NO.: 16) motif or RING-H2 domain. Wild type BCA1 was transcriptionally active when transformed into yeast, therefore making it unsuitable for yeast two-hybrid screening analysis. Thus, mutant BCA1 constructs were generated and used as the bait plasmid. Two Cys residues in the ring-finger domain were replaced by two Ser residues (mR) to generate one bait protein. The “Y” in the PPPPY (SEQ ID NO.: 16) motif was replaced by an “A” and two Cys residues in the ring-finger domain were replaced by two Ser residues (mRY40A) to generate another bait protein.

Selection of positive clones, indicating proteins that interact with the bait protein, yielded fifteen unique in-frame cDNAs (Table 1). Of these fifteen clones, the DNA sequence of two did not match to any sequence in Genbank, and three clones are hypothetical genes. EPS15, ZBRK1 and AMSH may have roles in signal transduction.

6.5.8 Protein Interactions with AIP4 and Smurf2

The PPPPY sequence of BCA1 is identical to that of LMP2A, which has been shown to bind the WW-domain binding motif of AIP4. AIP4 is an E3 ubiquitin ligase that mediates ubiquitination and degradation of the breast tumor suppressor Syk. A FLAG-BCA1 construct and a GST-AIP4 construct were co-transfected into HEK-293T cells. Total cell extracts were incubated with GST-AIP4, GST only, or whole cell extract, and precipitates analyzed by western blot analysis using an anti-FLAG antibody (FIG. 11B). The results demonstrate that GST-AIP4 can bind FLAG-BCA1.

A similar experiment was performed to examine the interaction between BCA1 and Smurf2. Cells were co-transfected with BCA1-FLAG and a GST-Smurf2 construct or a GST-Smurf2 mutant that cannot be ubiquitinated. Expression was confirmed by anti-FLAG western blot analysis. Antibody directed against Smurf2 co-precipitated both wild type and mutant Smurf2 with BCA-FLAG.

To test a possible function for Smurf2-BCA1 interaction, hemagluttinin (HA)-tagged ubiquitin was co-transfected with GST-BCA1 and/or FLAG-Smurf2. Precipitation of FLAG-tagged protein followed by western blot with anti-HA showed large amounts of HA-ubiquitin linked to FLAG-Smurf2. Little HA-ubiquitin was found on Smurf2 in the absence of BCA1. Therefore, the results indicate that BCA1 can interact with Smurf2 and the complex can interact with ubiquitin to a greater extent than uncomplexed Smurf2.

### 6.6. BCA2

6.6.1 BCA2 Nucleic Acid Sequence

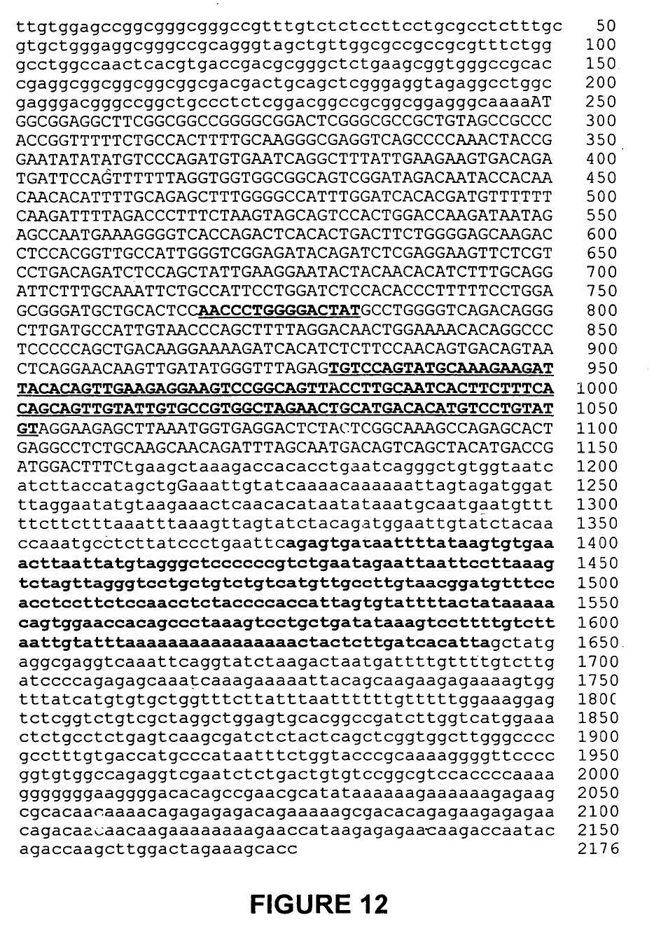

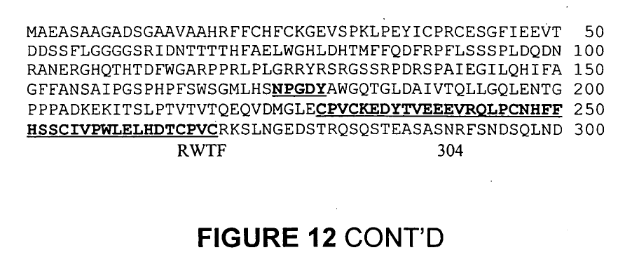

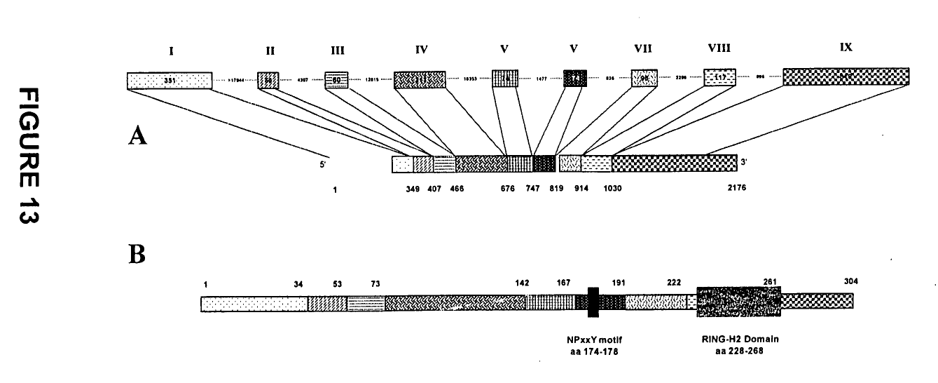

The sequence of BCA2 is shown in FIG. 12. The original 280-bp clone was extended using the Genbank database. The full length clone, RNF31, is a novel gene of 2176 bp. The 5′ end of the gene is delineated by the 5′ ends of EST BG684824 and EST BG685926, followed by AL530463 which extends as far as exon 8. At the 3′ end of RNF31, the EST BG028538 contains sequence contiguous with the sequence after bp 1746. A provisional intron/exon structure is presented in FIG. 13A showing nine predicted exons. Exon I (351 bp) is separated from exon II (60 bp) by 64,857 bases. Exon I is separated from exon III (60 bp) by 4307 bases. An intron of 12615 bases lies between exon III and exon IV (211 bp). An intron of 18354 bases lies between exon IV and V (74 bp). Exon V is followed by a 1478-bp intron, exon VI (74 bp), 935-bp intron, exon VII (96 bp), 2296-bp intron, exon VIII (117 bp), 996-bp intron, and finally exon IX (728 bp).

Exon VIII and exon IX each contain a RING-H2 domain. Exon VI contains an NPXXY domain. The exon I and II junction, exon IV and exon V each contain a PXXP motif. Additionally, a Zn-finger domain is located at the junction of exon I and II.

BCA2 has a RING-H2 domain distinct from that of BCA1. Further BCA2 has at least one consensus phosphorylation site sequence, NPGDY. Such motifs are known to mediate protein-protein interactions for proteins involved in signal transduction pathways and organization of cytoskeletal matrices. For example, ubiquitination is a highly regulated cellular process with E3 proteins having a central function. RING finger proteins constitute a large class of E3 proteins. Ring finger-containing E3 proteins have pivotal roles in diverse cellular processes linked to human disease, including the balance between proliferation and apoptosis, regulation of plasma membrane proteins, regulation of secretory pathways, and suppression of tumors as diverse as breast tumors and Von Hippel Lindau disease. Thus, the RING-H2 domain in BCA2 indicates a role in protein-protein interactions and can be involved in the ubiquitination pathway.

The ubiquitin-mediated proteolysis system is the major pathway for the selective, controlled degradation of intracellular proteins in eukaryotic cells. Targeted proteins undergoing selective degradation, through the actions of a ubiquitin-dependent proteosome, are covalently tagged with ubiquitin through the formation of an isopeptide bond between the C-terminal glycyl residue of ubiquitin and a specific lysyl residue in the substrate protein. This process is catalyzed by a ubiquitin-activating enzyme (E1), a ubiquitin-conjugating enzyme (E2), and may also require substrate recognition proteins (E3s). A number of E3 ubiquitin-protein ligases have previously been identified. See, e.g., D'Andrea et al., 1988, Nature Genetics, 18:97; Gonen et al., 1996, “Isolation, Characterization, and Purification of a Novel Ubiquitin-Protein Ligase, E3—Targeting of Protein Substrates via Multiple and Distinct Recognition Signals and Conjugating Enzymes”, J. Biol. Chem., 271:302; Scheffner et al., 1993, “The HPV-16 E6 and E6-AP Complex Functions as a Ubiquitin-Protein Ligase in the Ubiquitination of p53”, Cell 75:495; Huibregtse et al., 1995, “A Family of Proteins Structurally and Functionally Related to the E6-AP Ubiquitin Protein Ligase”, Proc Natl Acad Sci. 92:2563; Staub et al., 1996, “WW Domains of Nedd4 Bind to the Proline-Rich PY Motifs in the Epithelial Na+ Channel Deleted in Liddles Syndrome”, EMBO J. 15:2371 [the substrate specificity is determined by the E3 ligase]; Siepmann et al., 1996, “Evidence for Stable, Exchangeable E1/E2/E3 Ubiquitin Conjugation Complexes at Physiological Concentrations”, FASEB J., 10:2324.

Any techniques or methods useful for screening for compounds that bind the Ring domains of E3 proteins can be modified to screen for compounds that bind the Ring domain of the BCA polypeptides. Such compounds may have an inhibitory effect on the activity of the BCA polypeptides. Other E3 proteins have also been extensively evaluated in S. cerevisiae and in cell-free systems using engineered proteins as test substrates. See, e.g., Weissman, 1997, “Regulating Protein Degradation by Ubiquitination”, Review Immunology Today, 18(4):189; Sudakin et al., 1995, Mol. Biol. Cell 6:185; Stancovski et al., 1995, Mol. Cell. Biol. 15:7106; King et al., 1995, Cell, 81:279; Chen et al., 1996, Cell, 84:853; Orian et al., 1995, J. Biol. Chem. 170:21707; Varshavsky et al., 1992, Cell, 69:725; Hershko et al., 1992, Annu. Rev. Biochem. 61:761; Ciechanover, 1994, Cell 7:13. Such yeast systems and cell free systems can also be used to design binding assays for the BCA polypeptides of the invention.

Assays for screening for compounds that bind to the Ring finger domain of BRCA-1, can also be modified to screen for compounds that bind the Ring-H2 domains of the BCA polypeptides of the invention (see, e.g., U.S. Pat. No. 6,149,903).

6.6.2 Homologs of BCA2

A homology search for BCA2 revealed similar genes in mouse, fruit fly and yeast. The fruit fly gene product, CG11982, features a Zn-finger domain, NPXXY sequence and a RING-H2 domain, and exhibits 38% homology to BCA2, indicating that CG11982 may be a homologue of BCA2. Similarly, the fission yeast gene product, CAC29482, exhibits 39% similarity to BCA2, and may be a homologue of BCA2.

The sequence of the mouse, human and fruit fly all begin with the amino acid sequence MAEA indicating the importance of this region. Highest conservation exists in the region of the Zn-finger, NPDGYAWG, and RING-H2 domains. The NPXXY domain is identified in G-protein coupled receptors and may indicate a role for BCA2 in signal transduction or cytoskeletal matrix organization.

6.6.3 Immunohistochemistry of BCA2

BCA2 was cloned in-frame with the FLAG epitope and expressed in vitro. The fusion protein was visualized by anti-FLAG and anti-BCA2 antibodies raised against the C-terminus. Sections of breast tumor were stained using anti-BCA2 antibodies (FIG. 4A).

### 6.7. BCA3

6.7.1 BCA3 Nucleic Acid Sequence

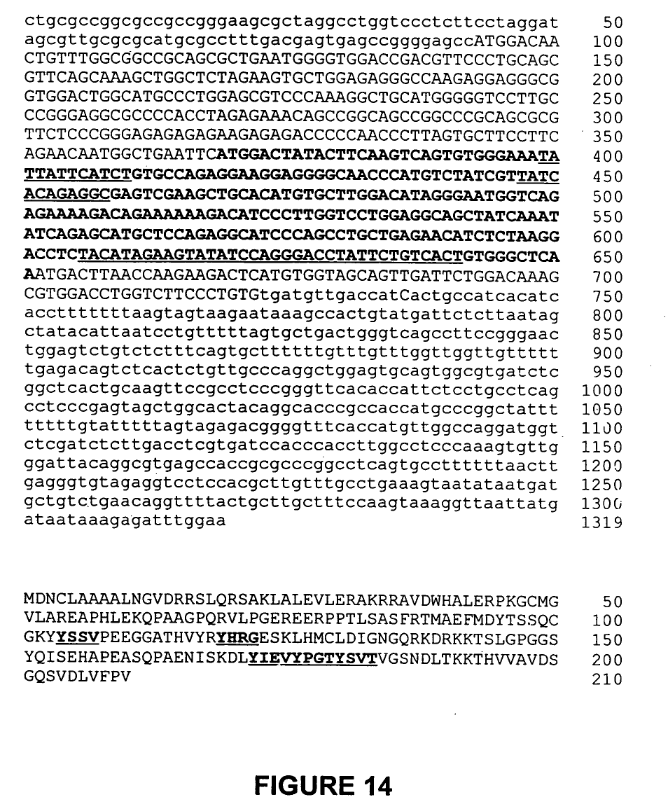

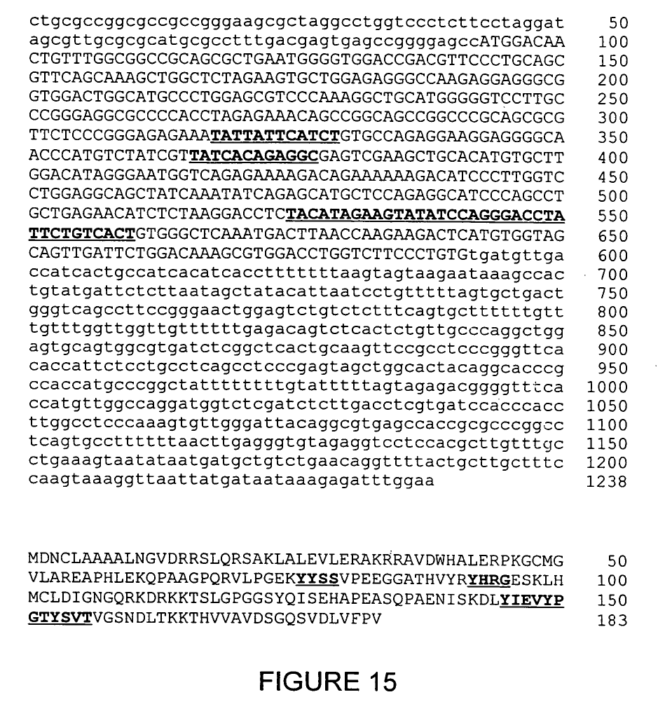

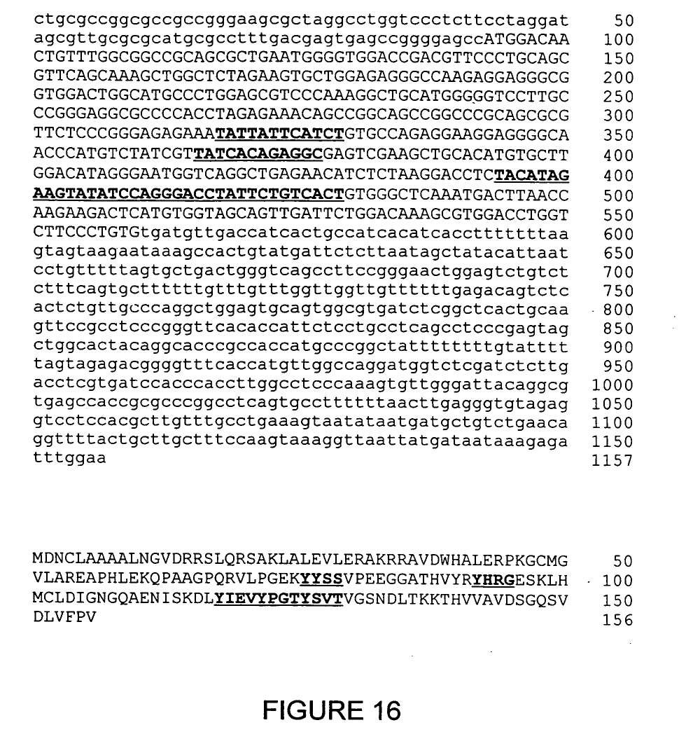

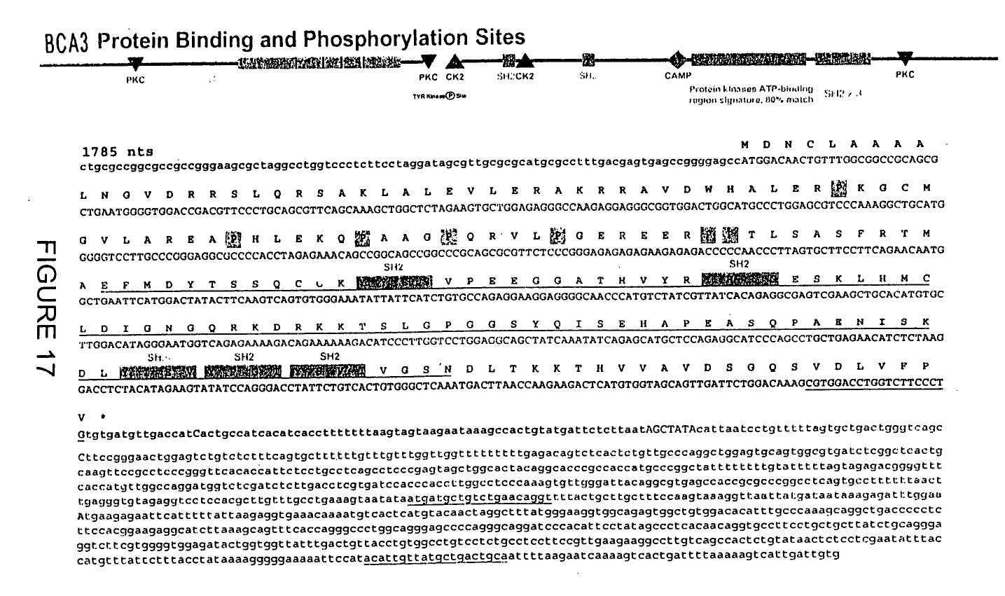

The BCA3 nucleotide sequence is shown in FIG. 14A. The initial clone of 322 bp was extended using overlapping EST sequences. The 5′ end of BCA3 is included in BI261767 and AA431064. The 3′ end is included in AI688557 (bp 1292 to 1756), AI799116 (ending at 1686), and AI202945 (ending at 1669). BCA3 is organized into 7 exons. Exon I (84 bp), exon 1(228 bp), exon III (81 bp), exon IV (105 bp), exon V (81 bp), exon VI (217 bp) and exon VII (884 bp) are separated by introns of 220, 779, 2294, 1969, 2355 and 29 bp, respectively. The final 1060 bps are found within AJ400877 separated from exon 6 by 29 bases. Genomic clone AL117342 has a region 88% similar to nucleotides 329-795, which includes exons 4, 5, and 6.

EST analysis revealed at least three different BCA3 mRNAs. After determining corresponding genomic sequences, the three mRNAs were determined to arise from alternative splicing, which was confirmed by RT-PCR, cloning and sequencing the alternatively spliced products (FIG. 18). A fourth splice variant appears to be expressed in SKBR3 breast cancer cells (FIG. 18).

The longest open-reading frame is translates into 210 amino acids having a proline-rich region in the amino terminal half and a series of consensus SH2-domain-binding motifs in the C-terminal region. The tyrosine phosphorylation motifs are in exon 3. Proline-rich sequences are located in exons 2, 3, 4, 5, and 6. Thus, BCA3 can be involved in signal transduction.

The proline-rich and SH2-binding regions in BCA3 are distinct and physically separated such that these domains are amenable to binding assays (e.g., yeast-two hybrid assays) designed to identify partner proteins. BCA3 also contains consensus sequences for interaction with protein kinase C, casein kinase 2 and cAMP.

6.7.2 BCA3 Homologs

A homology search for BCA3 has revealed an homologous open-reading frame in mouse. An open-reading frame with some homologous regions also exists in zebrafish.

6.7.3 BCA3 Antibody Preparation

Anti-BCA3 antibodies were prepared in rabbits against a peptide of the C-terminal sequence. The specificity of the antibodies was confirmed by western blot against in vitro expressed BCA3.

6.7.4 BCA3 Expression

BCA3 was placed into a pCMV vector and transfected into 293T cells. Lysates of the BCA3-transfected cells were subjected to gel electrophoresis and transferred to nitrocellulose. Western blots were probed with an anti-FLAG antibody and presence of BCA3 protein detected by chemiluminescence (FIG. 19C).

The TnT rabbit reticulocyte expression system was used to express BCA3 from pCMV-BCA3 (FIG. 19B). Constructs were prepared using three splice variants of BCA3 (i.e., full length, 552-bp variant (SEQ ID NO.:20), 471-bp variant(SEQ ID NO.:22)) SEQ ID NO.:, and each was expressed in vitro.

Full-length BCA3 appeared as a single band of 28 kDa. The 552-bp variant yielded an 18-kDa protein and the 471-bp variant produced a 16-kDa protein.

6.7.5 BCA3 Immunohistochemistry

Breast cancer sections were stained with anti-BCA3 antibody (FIG. 3).

### 7. INCORPORATION BY REFERENCE

All publications, patents and patent applications mentioned in this specification are herein incorporated by reference into the specification to the same extent as if each individual publication, patent or patent application was specifically and individually indicated to be incorporated herein by reference.

### 8. EQUIVALENTS

Those skilled in the art will recognize, or through routine experimentation, will be able to ascertain many equivalents to the particular embodiments of the invention described herein. The claimed invention intends to encompass all such equivalents.

Having herein above disclosed exemplary embodiments of the present invention, those skilled in the art will recognize that this disclosure is only exemplary such that various alternatives, adaptations, and modifications are within the scope of the invention, and are contemplated by the Applicants. Accordingly, the present invention is not limited to the specific embodiments as illustrated above, but is defined by the following claims.

1. An isolated polynucleotide comprising:
(a) the nucleotide sequence of the human BCA3 gene according to SEQ ID NO: 5;
(b) a nucleotide sequence that encodes a human BCA3 polypeptide;
(c) a nucleotide sequence that encodes the BCA3 polypeptide according to SEQ ID NO:6, 20, or 22;
(d) at least 12 consecutive bases of the human BCA3 gene, wherein said polynucleotide is not F29989, BG754249, BG654786, AU146189, AU145473, AV729000, AV725974, BE349302, BE205860, AW406755, AW339687, AI635272, AI365988, BM469324, BM558580, AW510839, BF337353, AA640772, AL599210, AL571890, BF913170, BE149796, BG681808, AA478355, BE304890, BI058894, BM042507, BG773327, AA521399, AA521323, AI873852, BI030630, BI023028, BG819532, BE909262, BE293845, BE293802, AW675725, AW193295, F19258, AI358229, AA478297, BG566176, AJ400877, NM_{—}020642, or BM449949; or
(e) a nucleotide sequence that is a complement of (d).

2. An isolated polynucleotide comprising a nucleotide sequence of 640 bases in length that hybridizes under highly stringent conditions to:
(a) a nucleotide sequence complementary to the coding region of the human BCA3 gene; or
(b) the nucleotide sequence of a human BCA3 mRNA.

3. An isolated polynucleotide comprising a nucleotide sequence encoding a fragment of the human BCA3 protein, wherein said fragment displays one or more functional activities.

4. An isolated polynucleotide comprising a BCA1 nucleotide sequence, herein said sequence is selected from the group consisting of residues 1-2659, 1-2500, 1-2000, 1-1500, 1-1000, 1-500, 1-124, 2516-2659, 2500-2659, 2000-2659, 1500-2659, 1000-2659, 500-2659, 124-2659, 363-377, 551-674 of SEQ ID NO: 1,5′-CCGCCGCCGCCATAT-3′ (SEQ ID NO.: 29), and 5′-TGTGTGATCTGTATGATGGACTTTGTTTATGGGGACCCAATTCGATTTCTGCCGTGCATGCACATCTATCACCTGGACTGTATAGATGACTGGTTGATGAGATCCTTCACGTGCCCCTCCTGC-3′ (SEQ ID NO.:30).

5. An isolated polynucleotide comprising a BCA2 nucleotide sequence, wherein said sequence is selected from the group consisting of residues 1-2176, 1-2000, 1-1500, 1-1000, 1-500, 1-100, 2000-2176, 1500-2176, 1000-2176, 500-2176, 100-2176, 768-782, 980-1052 of SEQ ID NO: 3, 5′-ATGGACAACTGTTTGGCGA-3′ (SEQ ID NO.:31), AGGGAAGACCAGGTCCACGC-3′ (SEQ ID NO: 46), 5′-AACCCTGGGGACTAT-3′ (SEQ ID NO: 47), 5′-AACCCTGGGGACTAT-3′ (SEQ ID NO.:32) and 5′-CCAAATGCCTCTTATCCCTGAATTCAGAGTGATAATTTTATAAGTGTGAAACTTAATTATGTAGGGCTCCCCCCGTCTGAATAGAATTAATTCCTTAAAGTCTAGTTAGGGTCCTGCTGTCTGTCATGTTGCCTTGTAACGGATGTTTCCACCTCCTTCTCCAACCTCTACCCCACCATTAGTGTATTTTACTATAAAAACAGTGGAACCACAGCCCTAAAGTCCTGCTGATATAAAGTCCTTTTGTCTTAATTGTATTTAAAAAAAAAAAAAAAACTACTCTTGATCACATTAGCTATG-3′ (SEQ ID NO.:33).

6. An isolated polynucleotide comprising a BCA3 nucleotide sequence, wherein said sequence is selected from the group consisting of residues 1-1756, 1-1686, 1-1500, 1-1000, 1-500, 1-100, 1686-1756, 1500-1756, 1000-1756, 500-1756, 100-1756, 399-410, 446-457, 606-617, 618-629, 630-641 of SEQ ID NO: 5, 5′-TATTATTCATCT-3′ (SEQ ID NO.:34), 5′-TATCACAGAGGC-3′ (SEQ ID NO.:35), 5′-TACATAGAAGTA-3′ (SEQ ID NO.:36), 5′-TATCCAGGGACC-3′ (SEQ ID NO.:37), and 5′-TATTCTGTCACT-3′ (SEQ ID NO.:38).

7. An isolated polynucleotide comprising a nucleotide sequence of at least 12 consecutive bases encoding a portion of a domain of a human BCA1 polynucleotide or polypeptide, wherein said domain is selected from the group consisting of a RING H2 finger, PY motif, glycosylation site, phosphorylation site, SH2-binding motif, open-reading frame, exon 1, exon 2, exon 3, intron 1, intron 2, 5′ untranslated region, and 3′ untranslated region.

8. An isolated polynucleotide comprising a nucleotide sequence of at least 12 consecutive bases encoding a portion of a domain of a human BCA2 polynucleotide or polypeptide, wherein said domain is selected from the group consisting of a RING H2, NPXXY motif, PXXP motif, zinc finger, glycosylation site, phosphorylation site, SH3-binding motif, open-reading frame, exon 1, exon 2, exon 3, exon 4, exon 5, exon 6, exon 7, exon 8, exon 9, intron 1, intron 2, intron 3, intron 4, intron 5, intron 6, intron 7, intron 8, 5′ untranslated region, and 3′ untranslated region.

9. An isolated polynucleotide comprising a nucleotide sequence of at least 12 consecutive bases encoding a portion of a domain of a human BCA3 polynucleotide or polypeptide, wherein said domain is selected from the group consisting of a SH2 site YYSS (SEQ ID NO.:39), SH2 site YSSV (SEQ ID NO.:40), SH2 site YHRG (SEQ ID NO.:41), SH2 site YIEV (SEQ ID NO.:42), SH2 site YPGT (SEQ ID NO.:43), SH2 site YSVT (SEQ ID NO.:44), tyrosine phosphorylation site, RTMAEFMDY (SEQ ID NO.:45), glycosylation site, phosphorylation site, tyrosine phosphorylation motif, SH2-binding motif, open-reading frame, open-reading frame lacking exon 3, open-reading frame lacking exon 3 and exon 5, exon 1, exon 2, exon 3, exon 4, exon 5, exon 6, exon 7, intron 1, intron 2, intron 3, intron 4, intron 5, intron 6, 5′ untranslated region, and 3′ untranslated region.

10. An isolated polynucleotide comprising a polynucleotide sequence encoding a chimeric protein, wherein said sequence is selected from the group consisting of the human BCA1, BCA2, BCA3, BCA4, BCA5, BCA6, and BCA7 gene.

11. An isolated RNA encoding a human BCA3 polypeptide.

12. A human BCA3 polypeptide comprising SEQ ID NO:6.

13. A human BCA1 polypeptide comprising SEQ ID NO:2.

14. A human BCA2 polypeptide comprising SEQ ID NO:4.

15. A fragment of a human BCA1 polypeptide comprising at least 5 consecutive amino acids of a human BCA1 polypeptide, wherein said fragment is a portion of a domain selected from the group consisting of a RING H2 finger, PY motif, glycosylation site, phosphorylation site, SH2-binding motif, open-reading frame, exon 1, exon 2, exon 3, intron 1, intron 2, 5′ untranslated region, and 3′ untranslated region.

16. A fragment of a human BCA2 polypeptide comprising at least 5 consecutive amino acids of a human BCA2 polypeptide, wherein said fragment is a portion of a domain selected from the group consisting of a RING H2, NPXXY motif, PXXP motif, zinc finger, glycosylation site, phosphorylation site, SH3-binding motif, open-reading frame, exon 1, exon 2, exon 3, exon 4, exon 5, exon 6, exon 7, exon 8, exon 9, intron 1, intron 2, intron 3, intron 4, intron 5, intron 6, intron 7, intron 8, 5′ untranslated region, and 3′ untranslated region.

17. A fragment of a human BCA3 polypeptide comprising at least 5 consecutive amino acids of a human BCA3 polypeptide, wherein said fragment is a portion of a domain selected from the group consisting of a SH2 site SH2 site YYSS (SEQ ID NO.:39), SH2 site YSSV (SEQ ID NO.:40), SH2 site YHRG (SEQ ID NO.:41), SH2 site YIEV (SEQ ID NO.:42), SH2 site YPGT (SEQ ID NO.:43), SH2 site YSVT (SEQ ID NO.:44), tyrosine phosphorylation site, RTMAEFMDY (SEQ ID NO.:45), glycosylation site, phosphorylation site, tyrosine phosphorylation motif, SH2-binding motif, open-reading frame, open-reading frame lacking exon 3, open-reading frame lacking exon 3 and exon 5, exon 1, exon 2, exon 3, exon 4, exon 5, exon 6, exon 7, intron 1, intron 2, intron 3, intron 4, intron 5, intron 6, 5′ untranslated region, and 3′ untranslated region.

18. A polypeptide comprising an amino acid sequence that has at least 90% identity to the fragment of claim 17, 18 or 19, wherein the percent identity is determined over an amino acid sequence of identical size to said fragment.

19. A complex comprising BCA1 polypeptide and a binding partner selected from the group consisting of a gene product of AIP4, Smurf2, polyubiquitin UbC, DUT, EPS15, ZBRK1, chromosome 19 open reading frame 5, AMSH, PLAT, TOM1L2, FLJ11626, clone 155, VIM, INVS, clone 287, clone 292, and POLR2J.

20. A polypeptide comprising an amino acid sequence that has 90% sequence identity relative to SEQ ID NO:2, and wherein said polypeptide binds to a binding partner selected from the group consisting of a gene product of AIP4, Smurf2, polyubiquitin UbC, DUT, EPS15, ZBRK1, chromosome 19 open reading frame 5, AMSH, PLAT, TOM1L2, FLJ11626, clone 155, VIM, INVS, clone 287, clone 292, and POLR2J.

21. An antibody that immunospecifically binds to a human BCA polypeptide, wherein said polypeptide is selected from the group consisting of BCA1, BCA2, BCA3, BCA4, BCA5, BCA6, and BCA7.

22. The antibody of claim 21 that immunospecifically binds to a human BCA polypeptide when bound to a binding partner, wherein said polypeptide is selected from the group consisting of BCA1, BCA2, BCA3, BCA4, BCA5, BCA6, and BCA7.

23. The antibody of claim 21 that immunospecifically binds to a human BCA polypeptide when bound to a binding partner; wherein said antibody does not bind to said polypeptide when not bound to said binding partner; and wherein said polypeptide is selected from the group consisting of BCA1, BCA2, BCA3, BCA4, BCA5, BCA6, and BCA7.

24. An expression vector comprising a human BCA polynucleotide, wherein said polynucleotide is selected from the group consisting of BCA1, BCA2, BCA3, BCA4, BCA5, BCA6, and BCA7.

25. A cell comprising a recombinant human BCA polynucleotide, wherein said polynucleotide is selected from the group consisting of BCA1, BCA2, BCA3, BCA4, BCA5, BCA6, and BCA7.

26. A transgenic non-human animal comprising a transgene that comprises a human BCA polynucleotide, wherein said polynucleotide is selected from the group consisting of BCA1, BCA2, BCA3, BCA4, BCA5, BCA6, and BCA7.

27. A method for making a BCA-3 polypeptide comprising the steps of:
(a) culturing a cell comprising a recombinant BCA-3 polynucleotide under conditions that allow said BCA-3 polypeptide to be expressed by said cell; and
(b) isolating the expressed BCA-3 polypeptide.

28. The product of the process of claim 27.

29. A method for preventing or treating breast cancer, said method comprising administering to a subject in need thereof an amount of a pharmaceutical composition comprising:
(a) a BCA polynucleotide;
(b) a BCA polypeptide; or
(c) an antibody that immunospecifically binds to a BCA polypeptide;
effective for preventing or treating said cancer, and a pharmaceutically acceptable carrier, wherein said polynucleotide or polypeptide is selected from the group consisting of a BCA1, BCA2, BCA3, BCA4, BCA5, BCA6, and BCA7 polynucleotide or polypeptide.

30. A method for preventing or treating breast cancer, said method comprising administering to a subject in need thereof an amount of:
(a) an expression vector comprising a human BCA polynucleotide; or
(b) an antisense BCA polynucleotide;
effective for preventing or treating said cancer, wherein said polynucleotide is selected from the group consisting of BCA1, BCA2, BCA3, BCA4, BCA5, BCA6, and BCA7.

31. A method for diagnosing a BCA-related disorder in a subject comprising the steps of:
(a) contacting a BCA antibody with a sample, suspected of containing a BCA polypeptide, from said subject under conditions that allow said BCA antibody to bind said BCA polypeptide; and
(b) detecting or measuring binding of said BCA antibody to said BCA polypeptide;
wherein said BCA polypeptide is selected from the group consisting of BCA1, BCA2, BCA3, BCA4, BCA5, BCA6, and BCA7; and
wherein said BCA-related disorder is determined to be present when the presence or amount of BCA polypeptide indicated by the detection or measurement of binding differs from a control value representing the amount of BCA polypeptide present in an analogous sample from a subject not having said BCA-related disorder.

32. A method for staging a BCA-related disorder in a subject comprising the steps of:
(a) contacting a BCA antibody with a sample, suspected of containing a BCA polypeptide, from said subject under conditions that allow said BCA antibody to bind said BCA polypeptide; and
(b) detecting or measuring binding of said BCA antibody to said BCA polypeptide;
wherein said BCA polypeptide is selected from the group consisting of BCA1, BCA2, BCA3, BCA4, BCA5, BCA6, and BCA7; and
wherein the stage of a BCA-related disorder in a subject is determined when the presence or amount of BCA polypeptide indicated by the detection or measurement of binding is compared with the amount of BCA polypeptide present in an analogous sample from a subject having a particular stage of a BCA-related disorder.

33. The method of claim 31 or 32, wherein said BCA-related disorder is breast cancer.

34. A method for identifying an analyte that binds a BCA polypeptide comprising the steps of:
(a) contacting said BCA polypeptide with an analyte under conditions that allow said analyte to bind said BCA polypeptide; and
(b) detecting binding of said BCA polypeptide to said analyte;
wherein said BCA polypeptide is selected from the group consisting of a BCA1, BCA2, BCA3, BCA4, BCA5, BCA6, and BCA7 polypeptide.

35. A method for identifying a protein that binds a BCA polypeptide comprising the steps of:
(a) contacting said BCA polypeptide with a positionally addressable array comprising a plurality of proteins, with each protein being at a different position on a solid support; and
(b) detecting binding of said BCA polypeptide to a protein on said array;
wherein said BCA polypeptide is selected from the group consisting of a BCA1, BCA2, BCA3, BCA4, BCA5, BCA6, and BCA7 polypeptide.

36. A method for identifying an analyte that binds a complex comprising a BCA polynucleotide or BCA polypeptide comprising the steps of:
(a) contacting said complex with said analyte under conditions that allow said analyte to bind said complex; and
(b) detecting binding of said BCA polynucleotide or BCA polypeptide to said analyte;
wherein said analyte binds to said BCA polynucleotide or BCA polypeptide when bound to said binding partner, and does not bind to said BCA polynucleotide or BCA polypeptide when not bound to said binding partner.

37. A method for identifying an analyte that inhibits formation of a complex comprising a BCA polynucleotide or BCA polypeptide comprising the steps of:
(a) contacting said complex with said analyte; and
(b) measuring the amount of said complex;
wherein a reduction in the amount of complex indicates that said analyte inhibits formation of said complex.

38. A method for identifying an inhibitor of growth of a breast cancer cell comprising the steps of:
(a) contacting said cell with:
(i) a BCA polynucleotide;
(ii) a BCA polypeptide; or
(iii) an antibody that immunospecifically binds to a BCA polypeptide;
(b) measuring cell growth;
wherein said polynucleotide or polypeptide is elected from the group consisting of a BCA1, BCA2, BCA3, BCA4, BCA5, BCA6, and BCA7 polynucleotide or polypeptide; and
wherein an inhibition of cell growth indicates the presence of an inhibitor of growth of a breast cancer cell.

39. A kit comprising, in a first container, a purified BCA nucleic acid, BCA polypeptide, BCA agonist, BCA antagonist, and in a second container, a molecule that binds to the BCA nucleic acid, BCA polypeptide, BCA agonist, BCA antagonist when bound to an analyte in a biological sample.
